(19) United States
(12) Patent Application Publication
(10) Pub. No.: US 2024/0093302 A1
(43) Pub. Date: Mar. 21, 2024

(54) NON-INVASIVE CANCER DETECTION BASED ON DNA METHYLATION CHANGES

(71) Applicant: NUCLEIX LTD., Rehovot (IL)

(72) Inventors: Danny FRUMKIN; Adam WASSERSTROM, Ness Ziona (IL); Revital KNIRSH, Rosh HaAyin (IL)

(73) Assignee: NUCLEIX LTD., Rehovot (IL)

(21) Appl. No.: 18/261,995

(22) PCT Filed: Jan. 18, 2022
(86) PCT No.: PCT/IL2022/050071

§ 371 Date: Jul. 18, 2023

(30) Foreign Application Priority Data
Jan. 19, 2021 (IL) 280297

(51) Int. Cl. C12Q 1/6886; C12Q 1/683

(52) U.S. Cl. CPC C12Q 1/6886; C12Q 1/683; C12Q 2600/112; C12Q 2600/154

(57) ABSTRACT

Methods and systems for assessing the presence of cancer in a subject and predicting the tissue source of the cancer are provided, by analyzing DNA methylation markers in cell- free DNA samples, particularly cell-free DNA from plasma samples.

### FIELD OF THE INVENTION

The present invention relates to methods and systems for assessing the presence of cancer in a subject and providing an indication of the tissue source of the cancer, by analyzing DNA methylation markers in cell-free DNA samples, particularly cell-free DNA from plasma samples.

### BACKGROUND OF THE INVENTION

Detection of tumors at the earliest possible stage is of paramount importance for cancer treatment. Currently, a limited number of screening tests are available for a few cancer types. Most cancer types currently lack an effective early screening option, let alone non-invasive screening. Blood-based universal cancer screening assays, that can detect the presence of multiple types of cancer in a single assay, are sought. More particularly, cancer detection assays based on genetic and/or epigenetic markers in body fluids which are indicative of multiple types of cancer are sought, with subsequent analysis of additional genetic and/or epigenetic markers to identify the tissue source of the cancer.

DNA methylation changes are known to occur in many types of cancer, including hypomethylation of isolated CpGs, and hypermethylation occurring mostly at CpG islands. Specifically, hypermethylation of CpG islands in the promotor regions of tumor suppressor genes, leading to gene silencing, has been studied extensively and demonstrated in many different types of cancer. Tumors release DNA fragments, or “cell-free DNA”, into body fluids and consequently methylation changes of tumor derived DNA molecules can be detected in “liquid biopsies” obtained from body fluids such as blood plasma and urine. In contrast to traditional biopsies, liquid biopsies are non-invasive and may better represent the full genetic spectrum of tumor sub-clones. Consequently, detection of methylation changes associated with cancer in liquid biopsies holds great promise for early detection, prognosis, and therapeutic surveillance. However, in order to detect tumor derived DNA in liquid biopsies, ultra-sensitive biochemical methods are required, because the tumor DNA can be present in extremely low quantities in relation to the large background of normal DNA.

Kang et al. (2017) Genome Biology, 18:53, report non-invasive cancer diagnosis and tissue-of-origin prediction using methylation profiles of cell-free DNA measured by whole-genome bisulfite sequencing.

Liu at al. (2020) Annals of Oncology, 31(6): 745-759, report multi-cancer detection and localization using methylation signatures in cell-free DNA. Plasma cfDNA underwent bisulfite sequencing targeting a panel of >100,000 informative methylation regions. A classifier was developed and validated for cancer detection and tissue of origin (TOO) localization.

Chen et al. (2020) Nature Communications, 11, Article number: 3475, report a blood-based cancer screening test that interrogates cancer-specific methylation signatures using bisulfite sequencing.

US 2009/0005268 discloses compositions and methods for cancer diagnostics, including but not limited to, pan-cancer markers. In particular, methods of identifying methylation patterns in genes associated with specific cancers are disclosed, and their related uses. In another aspect, methods of selecting and combining useful sets of pan-cancer markers are disclosed.

US 2018/0341745 discloses, inter alia, a method of diagnosing a cancer in an individual in need thereof, comprising: a) processing an extracted genomic DNA with a deaminating agent to generate a treated genomic DNA comprising deaminated nucleotides, wherein the extracted genomic DNA is obtained from a biological sample from the individual; b) generating a methylation profile of one or more biomarkers from the treated genomic DNA; and c) diagnosing whether the individual has a cancer by comparing the methylation profile to a reference CpG methylation profile obtained from a cancer CpG methylation profile database, wherein a correlation between the methylation profile and the reference CpG methylation profile determines the presence of cancer in the individual.

US 2020/0131582 discloses methods and systems of utilizing sequencing reads (methylation sequencing reads) for detecting and quantifying the presence of a tissue type or a disease type in cell-free DNA prepared from blood samples.

US 2020/0308651 discloses a method of detecting the presence of DNA from cancer cells in a subject comprising: providing a sample of cell-free DNA from a subject; subjecting the sample to library preparation to permit subsequent sequencing of the cell-free methylated DNA; adding a first amount of filler DNA to the sample, wherein at least a portion of the filler DNA is methylated, then optionally denaturing the sample; capturing cell-free methylated DNA using a binder selective for methylated polynucleotides; sequencing the captured cell-free methylated DNA; comparing the sequences of the captured cell-free methylated DNA to control cell-free methylated DNAs sequences from healthy and cancerous individuals and from individuals with distinct cancer types and subtypes; identifying the presence of DNA from cancer cells if there is a statistically significant similarity between one or more sequences of the captured cell-free methylated DNA and cell-free methylated DNAs sequences from cancerous individuals.

US 2019/0316209 discloses a predictive cancer model that generates a cancer prediction for an individual of interest by analyzing values of one or more types of features that are derived from cfDNA obtained from the individual. Specifically, cfDNA from the individual is sequenced to generate sequence reads using one or more physical assays, examples of which include a small variant sequencing assay, whole genome sequencing assay, and methylation sequencing assay. The sequence reads of the physical assays are processed through corresponding computational analyses to generate each of small variant features, whole genome features, and methylation features. The values of features can be provided to a predictive cancer model that generates a cancer prediction.

US 2020/0239965 discloses a method and system for determining one or more sources of a cfDNA test sample from a test subject. The cfDNA test sample contains a plurality of DNA molecules with numerous CpG sites that may be methylated or unmethylated. A trained deconvolution model comprises a plurality of methylation parameters, including a methylation level at each CpG site for each source, and a function relating a sample vector as input and a source of origin prediction as output. The method generates a test sample vector comprising a site methylation metric relating to DNA molecules from the test sample that are methylated at that CpG site. The method inputs the test sample vector into the trained deconvolution model to generate a source of origin prediction indicating a predicted DNA molecule contribution of each source.

WO 2020/154682 and WO 2020/163410 disclose a cancer assay panel for targeted detection of cancer-specific methylation patterns. Further disclosed are methods of designing, making, and using the cancer assay panel to detect cancer and particular types of cancer.

WO 2011/070441, assigned to the Applicant of the present invention, discloses a method for categorization of a DNA sample based on methylation differences, the method comprising: (A) digesting a DNA sample with a methylation-sensitive and/or methylation-dependent restriction endonuclease; (B) performing PCR on the digested DNA to co-amplify at least two genomic loci, of which at least one is a restriction locus differentially methylated between different DNA categories; (C) determining the intensity of the signal of each amplification product; (D) calculating signal ratios between the intensities of the signals produced by the loci; and (E) comparing the signal ratios to reference values corresponding to different categories of DNA, wherein the category whose reference values correspond best to the signal ratios is determined to be the category of the DNA sample. Categories of DNA samples include, for example, DNA from different tissues and/or physiological/pathological states.

WO 2017/006317 and WO 2019/142193, assigned to the Applicant of the present invention, disclose methods for identification of bladder cancer and methods for identification of lung cancer, respectively, based on alterations in DNA methylation at selected genomic loci. The methods involve calculating signal intensity ratios between selected loci co-amplified from a tested DNA sample following digestion with at least one methylation sensitive restriction enzyme, and comparing these ratios to one or more reference ratios.

WO 2020/188561, assigned to the Applicant of the present invention, discloses methods and systems for highly sensitive detection of methylation changes in DNA samples, particularly in DNA samples obtained from biological fluids such as plasma and urine, using methylation-sensitive/-dependent DNA digestion followed by target-specific PCR and/or next-generation sequencing. The high sensitivity is obtained when the analyzed DNA sample (namely, the DNA sample following extraction thereof from a biological sample) is substantially devoid of single-stranded DNA.

Hitherto described methods for multi-cancer screening have a number of drawbacks, such as multiple laborious steps, complicated analysis of numerous genomic loci and/or insufficient sensitivity and specificity to detect multiple types of cancers.

There is a need for improved non-invasive screening methods providing clinical performance required for a multi-cancer assay, including clinically useful sensitivity, high specificity and accurate prediction of tissue source of the cancer.

### SUMMARY OF THE INVENTION

The present invention provides methods and systems for assessing the presence of cancer in a human subject and providing an indication of the tissue source of the cancer, which are non-invasive, sensitive, specific and simple to use. More particularly, the present invention relates to DNA methylation markers that can be detected and analyzed in a cell-free DNA sample of a subject, which are indicative of the presence of cancer in the subject and of the tissue source of the cancer.

The present invention is based, in part, on the identification of three human genomic loci as DNA methylation markers for the detection of multiple cancer types in cell-free DNA samples. These genomic loci, set forth herein as SEQ ID NO: 1, SEQ ID NO: 2 and SEQ ID NO: 3, were found to have increased methylation levels in cell-free DNA of cancer patients of various types and stages compared to cell-free DNA of subjects with no cancer. The present invention is further based on the surprising finding that human genomic loci, set forth herein as SEQ ID NO: 4, SEQ ID NO: 5 and SEQ ID NO: 6, that were previously disclosed by the inventors of the present invention to be hypermethylated in lung cancer DNA compared to normal DNA, also have increased methylation levels in cancer types other than lung cancer, and are therefore useful as general markers of cancer. The inventors of the present invention identified that a certain increase in methylation levels of these marker loci is common to a variety of cancer types and stages, and thus serves as a general indication for the presence of cancer in a subject. A further increase in methylation levels is specifically indicative for the presence of lung cancer in the subject.

The set of marker loci set forth as SEQ ID NOs: 1-6 shows a remarkable sensitivity and specificity with respect to the detection of cancer. As exemplified below, when tested on a cohort of DNA samples comprising cell-free DNA samples from cancer patients of more than 10 different types of cancer at various stages, the set of markers showed an overall sensitivity (irrespective of cancer type and stage) of 62%, and an overall specificity of 94%. It is noted that the types of cancer that were tested account for the vast majority of cancer cases, and most of them do not have any screening option available at present, let alone non-invasive screening. Thus, with only six markers the presence of multiple types of cancer can be detected in a single assay.

In addition, and of great clinical significance, the set of markers was found to be particularly sensitive for early-stage cancers (stages I, II & IIIA), with over 40% sensitivity for stage I cancer, and over 50% sensitivity for stages I, II & IIIA. These results demonstrate the ability of the marker loci to detect the presence of various types of cancers at a stage at which treatment is more likely to be successful. Some of the cancer types that were tested are typically diagnosed only when significant symptoms are present and treatment options are limited.

The present invention is further based on the identification of a set of human genomic loci as DNA methylation markers distinguishing between lung cancer and four other cancer types: colorectal, liver, breast and hematological cancers. These genomic loci, set forth herein as SEQ ID NOs: 5, 8-15, are useful for providing an indication of the tissue source of the cancer in a subject identified as likely to have cancer. As exemplified herein below, the tissue-specificity marker loci of the present invention are able to distinguish between lung cancer and the four other cancer types with 88-95% accuracy.

The marker loci disclosed herein contain differentially methylated CG dinucleotides located within recognition sites of methylation-sensitive restriction endonucleases, and are therefore suitable for methylation analysis using methods such as methylation-sensitive enzymatic DNA digestion followed by quantitative PCR, and methylation-sensitive enzymatic DNA digestion followed by high-throughput sequencing.

According to one aspect, the present invention provides a method for assessing the presence of cancer in a human subject, the method comprising:
(a) determining in a cell-free DNA (cfDNA) sample of the subject a methylation value for at least one marker locus that is hypermethylated in cancer DNA compared to non-cancer DNA selected from the group consisting of SEQ ID NO: 1, SEQ ID NO: 2 and SEQ ID NO: 3; and
(b) comparing the methylation value of said at least one marker locus to at least one reference methylation value selected from a cancer methylation value and a non-cancer methylation value, to determine the likelihood that the subject has cancer.

In some embodiments, the cfDNA sample is cfDNA extracted from a plasma sample.

In some embodiments, the at least one marker locus comprises a plurality of marker loci selected from the group consisting of SEQ ID NO: 1, SEQ ID NO: 2 and SEQ ID NO: 3.

In some embodiments, step (a) comprises determining in the cfDNA sample of the subject a methylation value for each of the marker loci SEQ ID NO: 1, SEQ ID NO: 2 and SEQ ID NO: 3.

In some embodiments, step (a) further comprises determining in the cfDNA sample of the subject a methylation value for at least one marker locus that is hypermethylated in cancer DNA compared to non-cancer DNA selected from the group consisting of SEQ ID NO: 4, SEQ ID NO: 5 and SEQ ID NO: 6.

In some embodiments, step (a) comprises determining in the cfDNA sample of the subject a methylation value for each of the marker loci SEQ ID NO: 1, SEQ ID NO: 2, SEQ ID NO: 3, SEQ ID NO: 4, SEQ ID NO: 5 and SEQ ID NO: 6.

In some exemplary embodiments, the methylation value for SEQ ID NO: 1 is based at least on CG sites located between positions 3-95 of SEQ ID NO: 1. In certain additional embodiments, the methylation value for SEQ ID NO: 2 is based at least on CG sites located between positions 2-33 of SEQ ID NO: 2. In additional embodiments, the methylation value of SEQ ID NO: 3 is based at least on CG sites located between positions 2-58 of SEQ ID NO: 3.

In some exemplary embodiments, the methylation value for SEQ ID NO: 4 is based at least on CG sites located between positions 2-39 of SEQ ID NO: 4. In certain additional embodiments, the methylation value for SEQ ID NO: 5 is based at least on CG sites located between positions 2-63 of SEQ ID NO: 5. In additional embodiments, the methylation value of SEQ ID NO: 6 is based at least on CG sites located between positions 2-88 of SEQ ID NO: 6.

In some embodiments, the type of cancer the presence of which can be assessed comprises at least one of lung cancer, breast cancer, colorectal cancer, hepatocellular carcinoma (HCC), leukemia, lymphoma, esophageal cancer, gastric cancer, head and neck cancer, ovarian cancer, uterine cancer, pancreatic cancer and sarcoma.

In some embodiments, the method for assessing the presence of cancer comprises a step of subjecting the cfDNA sample to digestion with at least one methylation-sensitive restriction endonuclease recognizing a sequence within the at least one marker locus that is hypermethylated in cancer DNA compared to non-cancer DNA, thereby obtaining restriction endonuclease-treated DNA.

In some embodiments, the methylation value is determined by a methylation mapping comprising one or more analysis methods selected from the group consisting of DNA sequencing, real-time polymerase chain reaction, and array hybridization.

In some embodiments, determining the methylation value comprises, for a plurality of potential methylation sites in a plurality of marker loci, quantifying methylated read counts at the potential methylation sites and calculating a methylation value based on the number of methylated read counts.

In some embodiments, determining the methylation value comprises, for a plurality of marker loci, determining a ΔCq for each marker locus and calculating a single composite methylation value based on the ΔCq determined for each marker locus.

In some embodiments, the method for assessing the presence of cancer comprises: subjecting the cfDNA sample to digestion with at least one methylation-sensitive restriction endonuclease recognizing a sequence within the at least one marker locus that is hypermethylated in cancer DNA compared to non-cancer DNA, thereby obtaining restriction endonuclease-treated DNA;

co-amplifying from the restriction endonuclease-treated DNA the at least one marker locus and a control locus, thereby generating an amplification product for each locus; and

comparing a ratio between the signal intensities of the amplification products of each of said at least one marker locus and the control locus to at least one reference ratio selected from cancer reference ratio and non-cancer reference ratio, to determine likelihood that the subject has cancer.

In some embodiments, the step of subjecting the cfDNA sample to digestion with at least one methylation-sensitive restriction endonuclease is performed using a single methylation-sensitive restriction endonuclease. In some particular embodiments, the methylation-sensitive restriction endonuclease is HinP1I.

In some embodiments, the step of subjecting the cfDNA sample to digestion with at least one methylation-sensitive restriction endonuclease is performed using a plurality of methylation-sensitive restriction endonucleases. In some particular embodiments, the plurality of methylation-sensitive restriction endonucleases comprises HinP1I.

In some embodiments, the control locus is a locus that does not contain a nucleotide sequence recognized by the methylation-sensitive restriction endonuclease. In some particular embodiments, the at least one methylation-sensitive restriction endonuclease comprises HinP1I and the control locus is SEQ ID NO: 7.

In some embodiments, the cfDNA subjected to digestion with at least one methylation-sensitive restriction endonuclease comprises less than 1% ssDNA. In some embodiments, the cfDNA subjected to digestion with the at least one methylation-sensitive restriction endonuclease comprises less than 0.1% ssDNA. In some embodiments, the cfDNA subjected to digestion with the at least one methylation-sensitive restriction endonuclease comprises less than 0.01% ssDNA.

In some embodiments, the cfDNA subjected to digestion with the at least one methylation-sensitive restriction endonuclease comprises at least 3.5 ng cfDNA. According to other embodiments, the cfDNA subjected to digestion with the at least one methylation-sensitive restriction endonuclease comprises at least 3.5, 5, 7.5, or 10 ng cfDNA.

In some embodiment, the amplification product for each locus is between about 70 and about 180 bases in length. In some embodiment, the amplification product for each locus is between about 90 and about 150 bases in length.

In some embodiments, the restriction endonuclease-treated DNA comprises less than 1% ssDNA, contains at least 3.5 ng cfDNA, and the amplification product for each locus is between about 90 and about 150 bases in length.

In some embodiments, the step of co-amplifying from the restriction endonuclease-treated DNA the at least one marker locus and a control locus is performed using real-time PCR. In some embodiments, the step of co-amplifying from the restriction endonuclease-treated DNA the at least one marker locus and a control locus comprises adding fluorescent probes for assisting in detecting the amplification products of the at least one marker locus and the control locus. In some embodiments, the ratio between the signal intensities of the amplification products of each of said at least one marker locus and the control locus is calculated by determining the quantification cycle (Cq) for each locus and calculating 2^{(Cq control locus-Cq marker locus)}.

In some embodiments, the method for assessing the presence of cancer further comprises providing an indication of the tissue source of the cancer for a subject with a positive assessment of having cancer.

In some embodiments, providing an indication of the tissue source of the cancer comprises analyzing the methylation of at least one tissue-specificity marker locus selected from:
(A) one or more tissue specificity marker locus selected from SEQ ID NO: 5 and SEQ ID NO: 8;
(B) one or more tissue specificity marker locus selected from SEQ ID NO: 9 and SEQ ID NO: 10;
(C) one or more tissue specificity marker locus selected from SEQ ID NO: 11 and SEQ ID NO: 12; and
(D) one or more tissue specificity marker locus selected from SEQ ID NO: 13, SEQ ID NO: 14 and SEQ ID NO: 15. Each possibility of (A), (B), (C), or (D) represents a separate embodiment of the invention.

In some embodiments, analyzing the methylation of at least one tissue-specificity marker locus comprises:
(i) determining in the cfDNA sample a methylation value for the one or more tissue specificity marker locus distinguishing between lung cancer and colorectal cancer selected from SEQ ID NO: 5 and SEQ ID NO: 8;
(ii) comparing the methylation value of each of said one or more tissue specificity marker locus to a corresponding lung cancer reference methylation value and a corresponding colorectal cancer reference methylation value; and
(iii) determining, based on the comparison, whether the methylation value of the one or more tissue specificity marker locus determined for the cfDNA sample represents a lung cancer value or a colorectal cancer value.

In some embodiments, analyzing the methylation of at least one tissue-specificity marker locus comprises:
(i) determining in the cfDNA sample a methylation value for the one or more tissue specificity marker locus distinguishing between lung cancer and liver cancer selected from SEQ ID NO: 9 and SEQ ID NO: 10;
(ii) comparing the methylation value of each of said one or more tissue specificity marker locus to a corresponding lung cancer reference methylation value and a corresponding liver cancer reference methylation value; and
(iii) determining, based on the comparison, whether the methylation value of the one or more tissue specificity marker locus determined for the cfDNA sample represents a lung cancer value or a liver cancer value.

In some embodiments, analyzing the methylation of at least one tissue-specificity marker locus comprises:
(i) determining in the cfDNA sample a methylation value for the one or more tissue specificity marker locus distinguishing between lung cancer and breast cancer selected from SEQ ID NO: 11 and SEQ ID NO: 12;
(ii) comparing the methylation value of each of said one or more tissue specificity marker locus to a corresponding lung cancer reference methylation value and a corresponding breast cancer reference methylation value; and
(iii) determining, based on the comparison, whether the methylation value of the one or more tissue specificity marker locus determined for the cfDNA sample represents a lung cancer value or a breast cancer value.

In some embodiments, the analysis of the one or more tissue specificity marker locus distinguishing between lung cancer and breast cancer further comprises calculating a breast cancer score based on the methylation value of the one or more tissue specificity marker locus and gender information of the subject, and determining whether the cfDNA sample represents a lung cancer sample or a breast cancer sample based on the breast cancer score.

In some embodiments, analyzing the methylation of at least one tissue-specificity marker locus comprises:
(i) determining in the cfDNA sample a methylation value for the one or more tissue specificity marker locus distinguishing between lung cancer and hematological cancer selected from the group consisting of SEQ ID NO: 13, SEQ ID NO: 14 and SEQ ID NO: 15;
(ii) comparing the methylation value of each of said one or more tissue specificity marker locus to a corresponding lung cancer reference methylation value and a corresponding hematological cancer reference methylation value; and
(iii) determining, based on the comparison, whether the methylation value of the one or more tissue specificity marker locus determined for the cfDNA sample represents a lung cancer value or a hematological cancer value.

In some embodiments, step (i) comprises determining a methylation value for each tissue specificity marker locus and subsequently calculating a combined methylation value based on the individual methylation values, and following steps (ii) and (iii) are carried out using the combined methylation value.

In some embodiments, step (ii) comprises comparing to a threshold value differentiating between the tissue sources, and step (iii) comprises determining which tissue source the methylation value represents based on the methylation value being above or below the threshold.

In some embodiments, step (iii) comprises determining a likelihood score reflecting the likelihood that the cfDNA sample is of a particular tissue source based on the comparison to the corresponding reference values.

In some embodiments, analyzing the methylation of at least one tissue-specificity marker locus comprises:
(i) determining in the cfDNA sample a methylation value for each of the marker loci SEQ ID NO: 5, SEQ ID NO: 8, SEQ ID NO: 9, SEQ ID NO: 10, SEQ ID NO: 11, SEQ ID NO: 12, SEQ ID NO: 13, SEQ ID NO: 14 and SEQ ID NO: 15;
(ii) determining a plurality of likelihood scores comprising:
   a likelihood score reflecting the likelihood that the tissue source is lung cancer and not colorectal cancer based on comparison of the methylation values of the marker loci SEQ ID NO: 5 and SEQ ID NO: 8 to corresponding lung cancer and colorectal cancer reference methylation values;
   a likelihood score reflecting the likelihood that the tissue source is lung cancer and not liver cancer based on comparison of the methylation values of the marker loci SEQ ID NO: 9 and SEQ ID NO: 10 to corresponding lung cancer and liver cancer reference methylation values;
   a likelihood score reflecting the likelihood that the tissue source is lung cancer and not breast cancer based on comparison of the methylation values of the marker loci SEQ ID NO: 11 and SEQ ID NO: 12 to corresponding lung cancer and breast cancer reference methylation values;
   a likelihood score reflecting the likelihood that the tissue source is lung cancer and not a hematological cancer based on comparison of the methylation values of the marker loci SEQ ID NO: 13, SEQ ID NO: 14 and SEQ ID NO: 15 to corresponding lung cancer and hematological cancer reference methylation values; and
(iii) determining a lung cancer score based on the plurality of likelihood scores, reflecting the likelihood that the tissue source is lung cancer.

In some embodiments, the method further comprises preparing a report in paper or electronic form based on the assessment of the presence of cancer and optionally the indicated tissue source of the cancer, and optionally communicating the report to the subject and/or a healthcare provider of the subject.

According to another aspect, the present invention provides a method for profiling methylation of a cell-free DNA (cfDNA) sample of a human subject, the method comprising:
(a) determining in the cfDNA sample a methylation value for at least one marker locus that is hypermethylated in cancer DNA compared to non-cancer DNA selected from the group consisting of SEQ ID NO: 1, SEQ ID NO: 2 and SEQ ID NO: 3;
(b) determining for each of said at least one marker locus whether its methylation value represents a cancer methylation value or a non-cancer methylation value, based on a comparison to at least one reference methylation value selected from a cancer reference value and a non-cancer reference value, thereby profiling methylation of the cfDNA sample.

In some embodiments, step (a) further comprises determining in the cfDNA sample of the subject a methylation value for at least one marker locus that is hypermethylated in cancer DNA compared to non-cancer DNA selected from the group consisting of SEQ ID NO: 4, SEQ ID NO: 5 and SEQ ID NO: 6.

In some embodiments, the method for profiling methylation further comprises profiling methylation of one or more tissue-specificity marker loci, said profiling comprising at least one of:
(A) determining in the cfDNA sample a methylation value for at least one marker locus selected from SEQ ID NO: 5 and SEQ ID NO: 8, and determining for each of said at least one marker locus whether its methylation value represents a lung cancer methylation value or a colorectal cancer methylation value, based on a comparison to corresponding lung cancer and colorectal cancer reference values;
(B) determining in the cfDNA sample a methylation value for at least one marker locus selected from SEQ ID NO: 9 and SEQ ID NO: 10, and determining for each of said at least one marker locus whether its methylation value represents a lung cancer methylation value or a liver cancer methylation value, based on a comparison to corresponding lung cancer and liver cancer reference values;
(C) determining in the cfDNA sample a methylation value for at least one marker locus selected from SEQ ID NO: 11 and SEQ ID NO: 12, and determining for each of said at least one marker locus whether its methylation value represents a lung cancer methylation value or a breast cancer methylation value, based on a comparison to corresponding lung cancer and breast cancer reference values; and
(D) determining in the cfDNA sample a methylation value for at least one marker locus selected from SEQ ID NO: 13, SEQ ID NO: 14 and SEQ ID NO: 15, and determining for each of said at least one marker locus whether its methylation value represents a lung cancer methylation value or a hematological cancer methylation value, based on a comparison to corresponding lung cancer and hematological cancer reference values, thereby profiling methylation of the cfDNA sample.

According to yet another aspect, the present invention provides a method for profiling methylation of a cell-free DNA (cfDNA) sample of a human subject suspected of having cancer or at risk of having cancer, the method comprising determining in the cfDNA sample a methylation ratio for at least one marker locus selected from the group consisting of SEQ ID NO: 1, SEQ ID NO: 2 and SEQ ID NO: 3, wherein determining a methylation ratio comprises:
(a) subjecting the cfDNA to digestion with at least one methylation-sensitive restriction endonuclease recognizing a sequence within the at least one marker locus that is hypermethylated in cancer DNA compared to non-cancer DNA, thereby obtaining restriction endonuclease-treated DNA;
(b) co-amplifying from the restriction endonuclease-treated DNA the at least one marker locus and a control locus, thereby generating an amplification product for each locus;
(c) determining a signal intensity for each generated amplification product; and
(d) calculating a ratio between the signal intensities of the amplification products of each of said at least one restriction locus and the control locus, thereby measuring a methylation ratio for the at least one marker locus, thereby profiling methylation of the cfDNA sample.

In some embodiments, the method for profiling methylation further comprises determining in the cfDNA sample a methylation ratio for at least one marker locus selected from the group consisting of SEQ ID NO: 4, SEQ ID NO: 5 and SEQ ID NO: 6.

In some embodiments, the method for profiling methylation further comprises determining in the cfDNA sample a methylation ratio for at least one tissue-specificity marker locus selected from the group consisting of:
(A) one or more tissue specificity marker locus distinguishing between lung cancer and colorectal cancer selected from SEQ ID NO: 5 and SEQ ID NO: 8;
(B) one or more tissue specificity marker locus distinguishing between lung cancer and liver cancer selected from SEQ ID NO: 9 and SEQ ID NO: 10;
(C) one or more tissue specificity marker locus distinguishing between lung cancer and breast cancer selected from SEQ ID NO: 11 and SEQ ID NO: 12; and
(D) one or more tissue specificity marker locus distinguishing between lung cancer and hematological cancer selected from SEQ ID NO: 13, SEQ ID NO: 14 and SEQ ID NO: 15, thereby profiling methylation of the cfDNA sample.

According to some embodiments, the method further comprising a step of determining the stage of the cancer and optionally monitor the progression of the cancer, wherein the cancer surveillance and follow-up testing comprises an in-vitro analysis of one or more of blood tests, urine tests, cytology, imaging, and biopsy.

According to a further aspect, the present invention provides a method for treating or managing cancer in a subject in need thereof, the method comprising:
(a) determining in a cell-free DNA (cfDNA) sample of the subject a methylation value for at least one marker locus that is hypermethylated in cancer DNA compared to non-cancer DNA selected from the group consisting of SEQ ID NO: 1, SEQ ID NO: 2 and SEQ ID NO: 3, optionally further determining a methylation value for at least one marker locus that is hypermethylated in cancer DNA compared to non-cancer DNA selected from the group consisting of SEQ ID NO: 4, SEQ ID NO: 5 and SEQ ID NO: 6;
(b) determining a positive assessment for the presence of cancer in the subject based on a comparison of the methylation value of each of said at least one marker locus to at least one reference methylation value selected from cancer reference methylation value and non-cancer reference methylation value; and
(c) administering to the subject active cancer surveillance and follow-up testing to identify the tissue source of the cancer, determine stage of the cancer and optionally monitor the progression of the cancer, wherein the cancer surveillance and follow-up testing comprises one or more of blood tests, urine tests, cytology, imaging, endoscopy and biopsy.

In some embodiments, step (c) comprises administering whole-body PET-CT scan to a subject with a positive assessment for the presence of cancer.

According to a further aspect, the present invention provides a method for treating or managing cancer in a subject in need thereof, the method comprising:
(a) determining in a cell-free DNA (cfDNA) sample of the subject a methylation value for at least one marker locus that is hypermethylated in cancer DNA compared to non-cancer DNA selected from the group consisting of SEQ ID NO: 1, SEQ ID NO: 2 and SEQ ID NO: 3, optionally further determining a methylation value for at least one marker locus that is hypermethylated in cancer DNA compared to non-cancer DNA selected from the group consisting of SEQ ID NO: 4, SEQ ID NO: 5 and SEQ ID NO: 6;
(b) determining a positive assessment for the presence of cancer in the subject based on a comparison of the methylation value of each of said at least one marker locus to at least one reference methylation value selected from cancer reference methylation value and non-cancer reference methylation value;
(c) providing an indication of the tissue source of the cancer by analyzing the methylation of at least one tissue-specificity marker locus selected from: (A) one or more tissue specificity marker locus distinguishing between lung cancer and colorectal cancer selected from SEQ ID NO: 5 and SEQ ID NO: 8; (B) one or more tissue specificity marker locus distinguishing between lung cancer and liver cancer selected from SEQ ID NO: 9 and SEQ ID NO: 10; (C) one or more tissue specificity marker locus distinguishing between lung cancer and breast cancer selected from SEQ ID NO: 11 and SEQ ID NO: 12; and (D) one or more tissue specificity marker locus distinguishing between lung cancer and hematological cancer selected from SEQ ID NO: 13, SEQ ID NO: 14 and SEQ ID NO: 15; and
(d) administering to the subject active cancer surveillance and follow-up testing according to the indicated tissue source for definitive diagnosis of the cancer tissue source, staging of the cancer and optionally monitoring the progression of the cancer, wherein the cancer surveillance and follow-up testing comprises one or more of blood tests, urine tests, cytology, imaging, endoscopy and biopsy.

According to a further aspect, the present invention provides a method for treating or managing lung cancer in a subject in need thereof, the method comprising:
(a) determining in a cell-free DNA (cfDNA) sample of the subject a methylation value for at least one marker locus that is hypermethylated in cancer DNA compared to non-cancer DNA selected from the group consisting of SEQ ID NO: 1, SEQ ID NO: 2 and SEQ ID NO: 3, optionally further determining a methylation value for at least one marker locus that is hypermethylated in cancer DNA compared to non-cancer DNA selected from the group consisting of SEQ ID NO: 4, SEQ ID NO: 5 and SEQ ID NO: 6;
(b) determining a positive indication for the presence of cancer in the subject based on a comparison of the methylation value of each of said at least one marker locus to at least one reference methylation value selected from cancer reference methylation value and non-cancer reference methylation value;
(c) quantifying an increased risk for a presence of lung cancer in the subject by analyzing the methylation of at least one tissue-specificity marker locus selected from: (A) one or more tissue specificity marker locus distinguishing between lung cancer and colorectal cancer selected from SEQ ID NO: 5 and SEQ ID NO: 8; (B) one or more tissue specificity marker locus distinguishing between lung cancer and liver cancer selected from SEQ ID NO: 9 and SEQ ID NO: 10; (C) one or more tissue specificity marker locus distinguishing between lung cancer and breast cancer selected from SEQ ID NO: 11 and SEQ ID NO: 12; and (D) one or more tissue specificity marker locus distinguishing between lung cancer and hematological cancer selected from SEQ ID NO: 13, SEQ ID NO: 14 and SEQ ID NO: 15; and
(d) administering a computerized tomography (CT) scan to the subject with a quantified increased risk for the presence lung cancer.

In some embodiments, a method of treating or managing cancer according to the present invention further comprises administering treatment based on the tissue source and stage of the cancer, wherein the treatment comprises one or more of surgical resection, chemotherapy, radiation therapy, immunotherapy, and targeted therapy.

According to a further aspect, the present invention provides a method for quantifying cancer-related methylation changes in a cell-free DNA sample of a human subject, the method comprising:

determining in the cell-free DNA sample a methylation value for at least one marker locus selected from the group consisting of SEQ ID NO: 1, SEQ ID NO: 2 and SEQ ID NO: 3, optionally further determining a methylation value for at least one marker locus selected from the group consisting of SEQ ID NO: 4, SEQ ID NO: 5 and SEQ ID NO: 6,

thereby quantifying cancer-related methylation changes in the cell-free DNA sample, indicative of the presence of cancer in the subject.

In some embodiments, the method for quantifying cancer-related methylation changes further comprises quantifying cancer-related methylation changes in the cell-free DNA sample which are indicative of the tissue source of the cancer, said quantifying comprises at least one of:
(A) determining in the cfDNA sample a methylation value for at least one tissue specificity marker locus distinguishing between lung cancer and colorectal cancer selected from SEQ ID NO: 5 and SEQ ID NO: 8, and comparing to corresponding lung cancer and colorectal cancer reference values;
(B) determining in the cfDNA sample a methylation value for at least one marker locus distinguishing between lung cancer and liver cancer selected from SEQ ID NO: 9 and SEQ ID NO: 10, and comparing to corresponding lung cancer and liver cancer reference values;
(C) determining in the cfDNA sample a methylation value for at least one marker locus distinguishing between lung cancer and breast cancer selected from SEQ ID NO: 11 and SEQ ID NO: 12, and comparing to corresponding lung cancer and breast cancer reference values; and
(D) determining in the cfDNA sample a methylation value for at least one marker locus distinguishing between lung cancer and hematological cancer selected from SEQ ID NO: 13, SEQ ID NO: 14 and SEQ ID NO: 15, and comparing to corresponding lung cancer and hematological cancer reference values, thereby quantifying cancer-related methylation changes in the cell-free DNA sample, indicative of the tissue source of the cancer.

According to another aspect, the present invention provides a method for indicating a cancer tissue source in a human subject with a positive assessment of having cancer, the method comprising analyzing the methylation of at least one tissue-specificity marker locus selected from:
(A) one or more tissue specificity marker locus distinguishing between lung cancer and colorectal cancer selected from SEQ ID NO: 5 and SEQ ID NO: 8;
(B) one or more tissue specificity marker locus distinguishing between lung cancer and liver cancer selected from SEQ ID NO: 9 and SEQ ID NO: 10;
(C) one or more tissue specificity marker locus distinguishing between lung cancer and breast cancer selected from SEQ ID NO: 11 and SEQ ID NO: 12; and
(D) one or more tissue specificity marker locus distinguishing between lung cancer and hematological cancer selected from SEQ ID NO: 13, SEQ ID NO: 14 and SEQ ID NO: 15.

According to yet another aspect, the present invention provides a method for assessing the presence of cancer other than lung cancer in a human subject, the method comprising:
(a) determining in a cell-free DNA (cfDNA) sample of the subject a methylation value for at least one marker locus that is hypermethylated in cancer DNA compared to non-cancer DNA selected from the group consisting of SEQ ID NO: 1, SEQ ID NO: 2, SEQ ID NO: 3, SEQ ID NO: 4, SEQ ID NO: 5 and SEQ ID NO: 6;
(b) comparing the methylation value of each of said at least one marker locus to at least one reference methylation value selected from a cancer methylation value and a non-cancer methylation value, to determine the likelihood that the subject has cancer other than lung cancer.

According to another aspect, the present invention provides a method for assessing the presence of cancer in a human subject and providing an indication of the tissue source of the cancer, comprising:
(a) determining in a cell-free DNA (cfDNA) sample of the subject a methylation value for at least one marker locus that is hypermethylated in cancer DNA compared to non-cancer DNA selected from the group consisting of SEQ ID NO: 4, SEQ ID NO: 5 and SEQ ID NO: 6, and a methylation value for at least one tissue specificity marker locus selected from the group consisting of SEQ ID NOs: 5, 8-15;
(b) assessing the presence of cancer in the subject based on the methylation value of the at least one marker locus that is hypermethylated in cancer DNA compared to non-cancer DNA, wherein the assessment comprises comparing the methylation value of each of said at least one marker locus to at least one reference methylation value selected from a cancer methylation value and a non-cancer methylation value; and
(c) providing an indication of the tissue source of the cancer for a subject with a positive assessment of having cancer based on the methylation value of the at least one tissue specificity marker locus, wherein: SEQ ID NO: 5 and SEQ ID NO: 8 distinguish between lung cancer and colorectal cancer; SEQ ID NO: 9 and SEQ ID NO: 10 distinguish between lung cancer and liver cancer; SEQ ID NO: 11 and SEQ ID NO: 12 distinguish between lung cancer and breast cancer; and SEQ ID NO: 13, SEQ ID NO: 14 and SEQ ID NO: 15 distinguish between lung cancer and hematological cancer.

According to yet another aspect, the present invention provides use of a methylation value determined for at least one marker locus selected from the group consisting of SEQ ID NO: 1, SEQ ID NO: 2 and SEQ ID NO: 3 in a cell-free DNA sample of a human subject, for assessing the presence of cancer in the human subject.

In some embodiments, the use further comprises a methylation value determined for at least one marker locus selected from the group consisting of SEQ ID NO: 4, SEQ ID NO: 5 and SEQ ID NO: 6 in the cell-free DNA sample of the human subject.

According to yet another aspect, the present invention provides use of a methylation value determined for at least one marker locus selected from the group consisting of SEQ ID NOs: 5, 8-15 in a cell-free DNA sample of a human subject with a positive assessment of having cancer, for indicating the tissue source of the cancer, wherein: SEQ ID NO: 5 and SEQ ID NO: 8 distinguish between lung cancer and colorectal cancer; SEQ ID NO: 9 and SEQ ID NO: 10 distinguish between lung cancer and liver cancer; SEQ ID NO: 11 and SEQ ID NO: 12 distinguish between lung cancer and breast cancer; and SEQ ID NO: 13, SEQ ID NO: 14 and SEQ ID NO: 15 distinguish between lung cancer and hematological cancer.

According to yet another aspect, the present invention provides use of a marker locus comprising SEQ ID NO: 1, SEQ ID NO: 2 or SEQ ID NO: 3 for assessing the presence of cancer in a human subject. In some embodiments, methylation in cell-free DNA from the subject is assessed.

According to a yet another aspect, the present invention provides a method for treating lung, colon, breast, bladder, liver, or renal cell cancer in a patient, comprising:
(i) identifying the patient as being a lung, colon, breast, bladder, liver, or renal cell cancer patient by any of the methods described herein;
(ii) determining a mutational status of one or more genes selected from the group of KRAS, EGFR, ALK, ROS1, BRAF, RET, MET and NTRK in a tumor DNA sample from the patient; and
(iii) administering a therapy targeted to the mutational status of the patient's tumor DNA.

According to a yet another aspect, the present invention provides a cancer therapeutic for use in treating lung, colon, breast, bladder, liver, or renal cell cancer in a patient, comprising:
(i) identifying the patient as being a lung, colon, breast, bladder, liver, or renal cell cancer patient by any of the methods described herein;
(ii) determining a mutational status of one or more genes selected from the group of KRAS, EGFR, ALK, ROS1, BRAF, RET, MET and NTRK in a tumor DNA sample from the patient; and
(iii) administering a therapy targeted to the mutational status of the patient identified as being a lung, colon, breast, bladder, liver, or renal cell cancer patient.

According to a yet another aspect, the present invention provides a method for treating occult or stage 0 lung cancer in a patient, comprising:
(i) identifying the patient as being a lung cancer patient by any one of the methods described herein; and
(ii) administering active lung cancer surveillance to said patient, wherein the active surveillance comprises one or more of a pulmonary imaging study, a sputum cytology study, a bronchoalveolar lavage cytology study, a bronchothoracic biopsy, and a transthoracic biopsy.

According to a yet another aspect, the present invention provides a method for performing computerized tomography (CT) on a patient, comprising:
(i) identifying the patient as being a lung cancer patient by any one of the methods described herein; and
(ii) performing a low-dose computed tomography (LDCT) scan on the chest of the patient.

According to a yet another aspect, the present invention provides a method for performing computerized tomography (CT) on a patient, comprising:
(i) identifying the patient as being a lung cancer patient by any one of the methods described herein; and
(ii) performing a low-dose computed tomography (LDCT) scan on the chest of the patient identified as being a lung cancer patient.

According to a yet another aspect, the present invention provides a method for treating lung or breast cancer in a patient, comprising:
(i) identifying the patient as being a lung, breast, or colon cancer patient by any one of the methods described herein;
(ii) determining a PD-L1 status and optionally a mutational burden status in a tumor sample from the patient; and
(iii) assigning immunotherapy targeted to the PD-L1 status and optionally the mutational burden status of the tumor sample.

According to a yet another aspect, the present invention provides a method for treating a breast cancer patient, comprising:
(i) identifying the patient as being a breast cancer patient by any one of the methods described herein;
(ii) determining an ER, a HER2 and a BRCA1/2 status in a tissue sample from the patient; and
(iii) administering a therapy targeted to the ER, HER2 and BRCA1/2 status of the patient.

According to a yet another aspect, the present invention provides a method for treating a colon cancer patient, comprising:
(i) identifying the patient as being a colon cancer patient by any one of the methods described herein;
(ii) determining a mismatch repair (MMR) or microsatellite instability (MSI) status in a tumor sample from the patient; and
(iii) administering a therapy targeted to the MMR or MSI status of the patient.

It is to be understood that any combination of each of the aspects and the embodiments disclosed herein is explicitly encompassed within the disclosure of the present invention.

Further embodiments and the full scope of applicability of the present invention will become apparent from the drawings and detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE FIGURES

FIGS. 1A-1B. Testing pan-cancer methylation markers—cases and control cohorts. (1A) Demographic data; (1B) Cancer distributions of the cancer patients by type and stage. Cancer types in Training I: leukemia, lymphoma, breast, colorectal, esophageal, gastric, head& neck, HCC, lung, ovarian, uterine and pancreatic. Cancer types in Validation: leukemia, lymphoma, breast, colorectal, esophageal, gastric, head& neck, HCC, lung, ovarian, uterine, pancreatic and sarcoma.

FIGS. 2A-2B. Sensitivity and specificity of pan-cancer methylation markers. (2A) Validation cohort performance; (2B) Validation cohort sensitivity by cancer type and stage.

FIGS. 3A-3D. Performance of tissue-specificity methylation markers. (3A) Lung vs. colorectal; (3B) Lung vs. liver; (3C) Lung vs. breast; (3D) Lung vs. hematological cancers.

### DETAILED DESCRIPTION OF THE INVENTION

The present invention relates to systems and methods for assessing the presence of cancer in a human subject. The present invention further relates to methods and systems for providing an indication of the tissue source of the cancer. The methods and systems of the present invention are based on analyzing a plurality of DNA methylation markers in a cell-free DNA sample of subject, which are indicative of the presence of cancer in the subject and of the tissue source of the cancer. The term “plurality” as used herein refers to ‘at least two’ or ‘two or more’.

The methods of the present invention are particularly beneficial, as they provide highly sensitive and specific means for cancer screening which are non-invasive and user-independent. Remarkably, the methods disclosed herein provide sensitive detection of cancer in a subject based on cell-free DNA found in the subjects' plasma, despite the fact that plasma samples contain very low amounts of tumor-derived DNA.

The methods of the present invention further provide methods of treating cancer comprising identifying a subject as being a cancer patient (e.g., breast, lung, colon cancer) by determining a methylation value as described herein and any one of the methods described herein, and administering an appropriate therapy or monitoring.

Advantageously, the marker loci disclosed herein are suitable for methylation analysis using enzymatic digestion of DNA with at least one methylation-sensitive enzyme followed by real-time PCR of the marker loci and an internal reference locus, and subsequently precise quantification of ratios between the signals obtained from each marker locus and the signal of the internal reference locus. Thus, in some embodiments, methylation analysis according to the present invention does not require evaluating absolute methylation levels at the analyzed genomic loci, but rather calculating a signal ratio (reflecting the methylation ratio) between the analyzed genomic loci and an internal reference locus in the same sample. This is in contrast to conventional methods utilizing methylation analysis for distinguishing between tumor-derived and normal DNA, which require determining actual methylation levels at specific genomic loci. Thus, embodiments of the present invention eliminate the need for standard curves and/or additional laborious steps involved in determination of methylation levels per se, thereby offering a simple and cost-effective procedure. An additional advantage over known approaches for analyzing methylation is conferred by the signal ratios obtained according to some embodiments of the present invention, which are calculated between loci amplified in the same reaction mixture (i.e., under the same reaction conditions). This renders the methods insensitive to various “noise” factors, such as changes in template DNA concentration, PCR conditions, and presence of inhibitors. Such noises are inherent for methods that are based on quantifying methylation levels of loci by comparing signals from separate amplification reactions.

Methylation in the human genome occurs in the form of 5-methyl cytosine and is confined to cytosine residues that are part of the sequence CG, also denoted as CpG dinucleotides (cytosine residues that are part of other sequences are not methylated). Some CG dinucleotides in the human genome are methylated, and others are not. In addition, methylation is cell and tissue specific, such that a specific CG dinucleotide can be methylated in a certain cell and at the same time unmethylated in a different cell, or methylated in a certain tissue and at the same time unmethylated in different tissues. DNA methylation is an important regulator of gene transcription.

The methylation pattern of cancer DNA differs from that of normal DNA, wherein some loci are hypermethylated while others are hypomethylated. In some embodiments, the present invention provides methods and systems for sensitive detection of differentially methylated (e.g., hypermethylated) genomic loci associated with cancer.

DNA Sample

A DNA sample for use according to the present invention is cell-free DNA extracted from a biological fluid sample of a subject. The term “cell-free DNA” (abbreviated “cfDNA”) refers to DNA molecules which are freely circulating in body fluids and are not contained within intact cells. The origin of cfDNA is not fully understood but believed to be related to apoptosis, necrosis and active release from cells. cfDNA is released by both normal and tumor cells. cfDNA is highly fragmented, with an average length of approximately 150 base pairs. It is to be understood that the term “cell-free DNA” as used herein refers to DNA which is already cell-free in the body of the subject.

Biological fluid samples include plasma, serum, urine, cerebrospinal fluid, semen, stool, sputum and amniotic fluid. Each possibility represents a separate embodiment of the present invention. In particular embodiments, the cell-free DNA is from plasma. The term “plasma” refers to the liquid remaining after a whole blood sample is subjected to a separation process to remove the blood cells. Plasma samples for use according to the present invention may be samples separated from whole blood using any method of separation, including for example by centrifugation and/or filtration. Plasma samples for use according to the present invention may be collected using conventional collection containers or tubes.

A “subject” according to the present invention is typically a human subject. In some embodiments, the subject is suspected of having cancer. In additional embodiments, the subject is at risk of developing cancer, for example, based on age, previous history of cancer, genetic predisposition, and/or family history. In additional embodiments, the subject may exhibit suspicious clinical signs of cancer and/or is suspected of having cancer based on other prior assay(s) e.g., based on testing of other biomarker(s). In some embodiments, the subject is at risk of recurrence of cancer. In some embodiments, the subject shows at least one symptom or characteristic of cancer. In other embodiments, the subject is asymptomatic. In some embodiments, the subject was not previously diagnosed with cancer. In some embodiments, the subject was previously diagnosed and treated for cancer. In some embodiments, such a subject is in need of monitoring for the recurrence of the cancer.

Preferably, the DNA sample on which the methylation analysis is carried out is substantially free of single-stranded DNA (ssDNA). As used herein, “substantially free of ssDNA” or “substantially devoid of ssDNA” indicates a DNA sample in which less than 7% of the DNA is ssDNA, preferably less than 5% of the DNA is ssDNA, more preferably less than 1% of the DNA is ssDNA (namely, at least 99% of the DNA is double-stranded) (by number of molecules). In some embodiments, the DNA sample contains less than 0.1% ssDNA. In some embodiments, the DNA sample contains less than 0.01% ssDNA. In some embodiments, the DNA sample contains no ssDNA (free of ssDNA). Extraction of DNA to obtain a DNA sample substantially free of ssDNA is described, for example, in WO 2020/188561, assigned to the Applicant of the present invention. An exemplary kit for extracting cell-free DNA which is suitable for use with the methods of the present invention is QIAamp® Circulating Nucleic Acid Kit (QIAGEN, Hilden, Germany).

In some embodiments, all DNA that was extracted is used according to the present invention. In some embodiments, the DNA is not quantified prior to the methylation analysis according to the present invention. In other embodiments, the DNA is quantified prior to analysis thereof. In some embodiments, the DNA is aliquoted, e.g., into a first aliquot that is subjected to a certain treatment and a second aliquot that is kept as an untreated control.

Methylation Value

A “methylation value” as used herein is a numerical value representing the level of methylation of a particular genomic locus in a DNA sample. As methylation may be analyzed and measured using various methods, a “methylation value” according to the present invention may be expressed as a variety of numerical values. For example, a methylation value may be a methylation level expressed as a ratio or percentage of the DNA molecules that are methylated at a marker locus out of the total number of DNA molecules containing the marker locus in the sample. As a further example, a methylation value may be a methylation level expressed as a copy number of methylated DNA molecules at the marker locus (e.g., read count obtained following sequencing, as will be described in more detail below). As yet a further example, a methylation value may be a methylation level expressed as an intensity of a signal obtained from a marker locus, e.g., fluorescent signal obtained using a detectable fluorescent label/probe. A methylation value may be normalized with respect to a reference locus and/or a reference DNA sample. In some particular embodiments, the methylation value is a methylation ratio between a marker locus and a control locus, expressed as a ratio between signals obtained for these loci following methylation-sensitive enzymatic digestion of the DNA sample and PCR amplification, as will be described in more detail below.

As described herein, each marker locus of the present invention contains a plurality of differentially methylated CG dinucleotides located within restriction sites of methylation-sensitive restriction endonucleases. In some embodiments, when the marker loci are analyzed using methylation-sensitive enzymatic digestion of the DNA, the methylation value for a marker locus is based on CG dinucleotides within restriction site(s) of the methylation-sensitive restriction endonuclease(s) used in the assay.

The following sections describe methylation analyses according to some embodiments of the present invention, which are based on methylation-sensitive enzymatic digestion of the DNA sample followed by quantitative PCR amplification and analysis of amplification products, or methylation-sensitive enzymatic digestion of the DNA sample followed by high-throughput sequencing (next-generation sequencing). A person of skill in the art would appreciate that other methods for analyzing methylation and obtaining methylation value(s) of marker loci may be used with the present invention.

Methylation Analysis Using Methylation-Sensitive Enzymatic Digestion and PCR Amplification

A. DNA Digestion

According to some embodiments of the present invention, following extraction the DNA is subjected to digestion with at least one methylation-sensitive restriction endonuclease. For example, one, two or three methylation-sensitive restriction endonucleases may be used. Each number of endonucleases used in the assay represents a separate embodiment of the present invention.

In some embodiments, the entire DNA that was extracted is used in the digestion step. In some embodiments, the DNA is not quantified prior to being subjected to digestion. In other embodiments, the DNA is quantified prior to digestion thereof. In some embodiments, the DNA is aliquoted into a first aliquot that is subjected to digestion and a second aliquot that is kept as an undigested control.

A “restriction endonuclease”, used herein interchangeably with a “restriction enzyme”, refers to an enzyme that cuts DNA at or near specific recognition sequences, also known as restriction sites. Restriction sites are usually 4 to 8 nucleotide long and are typically palindromic (i.e., the DNA sequences are the same in both directions).

A “methylation-sensitive” restriction endonuclease is a restriction endonuclease that cleaves its recognition sequence only if it is unmethylated (while methylated sites remain intact). Thus, the extent of digestion of a DNA sample by a methylation-sensitive restriction endonuclease depends on the methylation level, where a higher methylation level protects from cleavage and accordingly results in less digestion.

Examples of methylation-sensitive restriction endonucleases that may be used according to the present invention include AciI, BstuI, HhaI, HinP1I, HpaII, HpyCH4VI and combinations thereof. Each possibility represents a separate embodiment of the present invention.

In general, embodiments which can be performed with methylation-sensitive restriction endonuclease(s) can be done alternatively with methylation-dependent restriction endonucleases, and downstream steps will be adjusted accordingly. Examples of methylation-dependent restriction endonucleases that may be used according to the present invention include McrBc, MspJI and a combination thereof.

In some embodiments, a DNA sample according to the present invention is subjected to digestion with a single methylation-sensitive restriction endonuclease. In additional embodiments, a DNA sample according to the present invention is subjected to digestion with a plurality of methylation-sensitive restriction endonucleases, for example, two methylation-sensitive restriction endonucleases. In some embodiments, the plurality of methylation-sensitive restriction endonucleases comprises HinP1I. In additional embodiments, the plurality of methylation-sensitive restriction endonucleases comprises HhaI. In additional embodiments, the plurality of methylation-sensitive restriction endonucleases comprises AciI. In some particular embodiments, HinP1I and AciI are used. In some embodiments, DNA digestion may be carried out to complete digestion.

Complete digestion may be achieved following one to two hours incubation with the enzyme(s) at 37° C.

B. Amplification of Genomic Loci

The terms “genomic locus” and “locus” as used herein are interchangeable and refer to a DNA sequence at a specific position within the genome. The specific position may be identified by the molecular location, namely, by the chromosome and the numbers of the starting and ending base pairs on the chromosome. A variant of a DNA sequence at a given genomic position is called an allele. Alleles of a locus are located at identical sites on homologous chromosomes. Genomic loci include gene sequences as well as other genetic elements (e.g., intergenic sequences).

A “marker locus” as disclosed herein refers to a genomic locus that is differentially methylated between sources of DNA, and therefore analysis of its methylation provides an indication with respect to the source of the DNA.

Marker loci disclosed herein include pan-cancer marker loci, which are genomic loci differentially methylated between normal and cancer DNA of multiple types and stages, and are therefore useful as general markers of cancer. The pan-cancer marker loci of the present invention, set forth as SEQ ID NOs: 1-6 as detailed below, are hypermethylated in cancer DNA compared to normal (non-cancer) DNA, meaning that these genomic loci contain CG dinucleotides that are more methylated in cancer DNA compared to normal non-cancer DNA of various types and stages.

The marker loci set forth as SEQ ID NO: 4, SEQ ID NO: 5 and SEQ ID NO: 6 were previously disclosed by the inventors of the present invention to be hypermethylated in lung cancer DNA compared to normal DNA (WO 2019/142193, assigned to the Applicant of the present invention). It is now disclosed that these marker loci also have increased methylation levels in cancer types other than lung cancer. More particularly, the inventors of the present invention identified that methylation levels of these marker loci above a first threshold are common to a variety of cancer types and stages, and thus serve as a general indication for the presence of cancer in a subject. A further increase in methylation levels, above a second threshold, is seen specifically in lung cancer, and thus indicative for the presence of lung cancer in the subject.

The types of cancer the presence of which can be detected by analyzing the pan-cancer marker loci disclosed herein include lung cancer, breast cancer, colorectal cancer, hepatocellular carcinoma (HCC), leukemia, lymphoma, esophageal cancer, gastric cancer, head& neck cancer, ovarian cancer, uterine cancer, pancreatic cancer and sarcoma. Each possibility represents a separate embodiment of the present invention.

A pan-cancer marker locus, or pan-cancer marker loci, according to the present invention include any one or more of the genomic loci set forth in SEQ ID NOs: 1-6, as follows (positions are provided based on hg18 genomic build):
SEQ ID NO: 1, corresponds to position 63134659-63134754 on chromosome 2;
SEQ ID NO: 2, corresponds to position 22582059-22582092 on chromosome 10;
SEQ ID NO: 3, corresponds to position 62523441-62523499 on chromosome 19;
SEQ ID NO: 4, corresponds to position 176712765-176712804 on chromosome 2;
SEQ ID NO: 5, corresponds to position 44151843-44151906 on chromosome 17;
SEQ ID NO: 6, corresponds to position 168907231-168907319 on chromosome 1.

Methylation analysis of SEQ ID NO: 1 is based at least on CG sites located between positions 3-95 of SEQ ID NO: 1. For example, one or more CG sites corresponding to positions 63134661, 63134666, 63134672, 63134682, 63134692, 63134703, 63134706, 63134712 and 63134752 on chromosome 2.

Methylation analysis of SEQ ID NO: 2 is based at least on CG sites located between positions 2-33 of SEQ ID NO: 2. For example, one or more CG sites corresponding to positions 22582060, 22582066 and 22582090 on chromosome 10.

Methylation analysis of SEQ ID NO: 3 is based at least on CG sites located between positions 2-58 of SEQ ID NO: 3. For example, one or more CG sites corresponding to positions 62523442, 62523445, 62523475 and 62523497 on chromosome 19.

Methylation analysis of SEQ ID NO: 4 is based at least on CG sites located between positions 2-39 of SEQ ID NO: 4. For example, one or more CG sites corresponding to positions 176712766, 176712771, 176712775, 176712786, 176712788 and 176712802 on chromosome 2.

Methylation analysis of SEQ ID NO: 5 is based at least on CG sites located between positions 2-63 of SEQ ID NO: 5. For example, one or more CG sites corresponding to positions 44151844, 44151849, 44151874, 44151888 and 44151904 on chromosome 17.

Methylation analysis of SEQ ID NO: 6 is based at least on CG sites located between positions 2-88 of SEQ ID NO: 6. For example, one or more CG sites corresponding to positions 168907232, 168907247, 168907299, 168907301 and 168907317 on chromosome 1.

Marker loci disclosed herein also include tissue-specificity marker loci. Tissue-specificity marker loci according to the present invention are differentially methylated between different cancerous tissue sources. For example, DNA from a first cancerous tissue source, e.g., lung cancer, may have an increased methylation level at tissue-specificity marker loci of the present invention compared to DNA from a second cancerous tissue source, e.g., liver cancer. More particularly, the tissue-specificity maker loci contain CG dinucleotides that are differentially methylated between different cancerous tissue sources.

The tissue-specificity marker locus, or tissue-specificity marker loci, according to the present invention include any one or more of the genomic loci set forth in SEQ ID NOs: 5, 8-15, as follows (positions are provided based on hg18 genomic build):
SEQ ID NO: 5, corresponds to position 44151843-44151906 on chromosome 17, hypermethylated in lung cancer vs. colorectal cancer;
SEQ ID NO: 8, corresponds to position 27171718-27171744 on chromosome 7, hypermethylated in lung cancer vs. colorectal cancer;
SEQ ID NO: 9, corresponds to position 13646665-13646721 on chromosome 11, hypermethylated in liver cancer vs. lung cancer;
SEQ ID NO: 10, corresponds to position 45405239-45405318 on chromosome 17, hypermethylated in lung cancer vs. liver cancer;
SEQ ID NO: 11, corresponds to position 76774710-76774756 on chromosome 4, hypermethylated in breast cancer vs. lung cancer;
SEQ ID NO: 12, corresponds to position 29458817-29458881 on chromosome 1, hypermethylated in lung cancer vs. breast cancer;
SEQ ID NO: 13, corresponds to position 226713528-226713595 on chromosome 1, hypermethylated in hematological cancer vs. lung cancer;
SEQ ID NO: 14, corresponds to position 108662393-108662450 on chromosome 9, hypermethylated in hematological cancer vs. lung cancer;
SEQ ID NO: 15, corresponds to position 11234673-11234737 on chromosome 16, hypermethylated in lung cancer vs. hematological cancer.

Methylation analysis of SEQ ID NO: 8 is based at least on CG sites located between positions 2-71 of SEQ ID NO: 8. For example, one or more CG sites corresponding to positions 27171719, 27171723, 27171725, 27171742 and 27171787 on chromosome 7.

Methylation analysis of SEQ ID NO: 9 is based at least on CG sites located between positions 2-56 of SEQ ID NO: 9. For example, one or more CG sites corresponding to positions 13646666, 13646680, 13646699, 13646716, 13646719 on chromosome 11.

Methylation analysis of SEQ ID NO: 10 is based at least on CG sites located between positions 2-79 of SEQ ID NO: 10. For example, one or more CG sites corresponding to positions 45405240, 45405242, 45405272, 45405300, 45405316 on chromosome 17. Methylation analysis of SEQ ID NO: 11 is based at least on CG sites located between positions 2-46 of SEQ ID NO: 11. For example, one or more CG sites corresponding to positions 76774711, 76774713, 76774731, 76774743 and 76774754 on chromosome 4.

Methylation analysis of SEQ ID NO: 12 is based at least on CG sites located between positions 2-64 of SEQ ID NO: 12. For example, one or more CG sites corresponding to positions 29458818, 29458853, 29458855, 29458875, 29458877 and 29458879 on chromosome 1.

Methylation analysis of SEQ ID NO: 13 is based at least on CG sites located between positions 2-67 of SEQ ID NO: 13. For example, one or more CG sites corresponding to positions 226713529, 226713535, 226713573 and 226713593 on chromosome 1.

Methylation analysis of SEQ ID NO: 14 is based at least on CG sites located between positions 2-57 of SEQ ID NO: 14. For example, one or more CG sites corresponding to positions 108662394, 108662446 and 108662448 on chromosome 9.

Methylation analysis of SEQ ID NO: 15 is based at least on CG sites located between positions 2-64 of SEQ ID NO: 15. For example, one or more CG sites corresponding to positions 11234674, 11234678, 11234685, 11234692, 11234720, 11234729, 11234731 and 11234735 on chromosome 16.

The inventors of the present invention have advantageously identified that the pan-cancer marker loci and the tissue-specificity marker loci can be detected in cell-free DNA, particularly in cfDNA from plasma samples, enabling non-invasive cancer detection and characterization.

In some embodiments, a pan-cancer marker locus according to the present invention contains a plurality of CG dinucleotides which are more methylated in cell-free DNA from plasma samples of subjects with cancer than in cell-free DNA from plasma samples of healthy subjects. In some embodiments, plasma samples of the cancer patients contain a greater number of DNA molecules that are methylated at the pan-cancer marker loci compared to plasma samples of healthy subjects.

In some embodiments, a tissue-specificity marker locus according to the present invention contains a plurality of CG dinucleotides which are differentially methylated between cell-free DNA from plasma samples of subjects with a certain type of cancer and cell-free DNA from plasma samples of subjects with a different type of cancer. In some embodiments, plasma samples of the cancer patients with a first type of cancer contain a greater number of DNA molecules that are methylated at the tissue-specificity marker locus compared to plasma samples of cancer patients with a second type of cancer.

Advantageously, the marker loci disclosed herein contain differentially methylated CG dinucleotides located within recognition site(s) of at least one methylation-sensitive restriction enzyme. A methylation-sensitive restriction enzyme cleaves its recognition sequence only if it is unmethylated. Thus, differences in methylation levels between DNA sources result in differences in the degree of digestion, and subsequently different amplification patterns in subsequent amplification and quantification steps. Such differences enable distinguishing between DNA from different sources, for example, between DNA samples from subjects with cancer and DNA samples from healthy subjects.

A “control locus” and “internal reference locus” are interchangeable and used herein to describe a locus, the digestion of which with the restriction enzyme applied in the digestion step is independent of the presence or absence of methylation. In some embodiments, the control locus is a locus devoid of the nucleotide sequence recognized by the at least one restriction enzyme applied in the digestion step, and the sequence of the control locus remains intact regardless of its methylation status when the DNA sample is digested. Thus, the sequence of the control locus exhibits the same digestion and amplification pattern in DNA from different sources, e.g., in normal and cancer DNA. Advantageously, the control locus is an internal locus, i.e., a locus within the analyzed DNA sample, thus eliminating the need for external/additional control sample(s).

One advantage in using the pan-cancer marker loci set forth in SEQ ID NOs: 1-6 for detecting the presence of cancer, and using the tissue-specificity marker loci set forth in SEQ ID NOs: 5, 8-15 for differentiating between lung cancer and four (4) other types of cancer (liver, colorectal, breast and hematological cancers) is exemplified herein below using the methylation sensitive restriction enzymes HinP1I and AciI. Amplification of marker loci comprising each of SEQ ID NOs: 1-6 and of a control locus comprising SEQ ID NO: 7 (that does not contain the recognition sequence of HinP1I and AciI) following digestion with HinP1I and AciI was carried out in plasma DNA from cancer patients and healthy individuals.

The panel of pan-cancer markers showed an overall sensitivity (irrespective of cancer type and stage) of 62%, and an overall specificity of 94%. Highest sensitivity was demonstrated in GI cancers (77% colorectal, 83% esophageal, 100% gastric) and non-solid malignancies (83%). The panel of pan-cancer markers was particularly sensitive for early-stage cancers (stages I, II & IIIA), with above 40% sensitivity for stage I cancer, and 52% sensitivity for stages I, II & IIIA.

The tissue-specificity markers correctly identified the tissue source of the cancer with 88-95% accuracy.

In some embodiments, the methods of the present invention comprise amplifying at least one marker locus and at least one control locus following digestion of the DNA sample.

As used herein, “amplification” refers to an increase in the number of copies of one or more particular nucleic acid target of interest. Amplification is typically performed by polymerase chain reaction (PCR) in the presence of a PCR reaction mixture which may include a suitable buffer supplemented with the DNA template, polymerase (usually Taq Polymerase), dNTPs, primers and probes (as appropriate).

The term “polynucleotide” as used herein include polymeric forms of nucleotides of any length, either deoxyribonucleotides or ribonucleotides, or analogs thereof. The term “oligonucleotide” is also used herein to include a polymeric form of nucleotides, typically of up to 100 bases in length.

An “amplification product” collectively refers to nucleic acid molecules of a particular target sequence that are generated and accumulated in an amplification reaction. The term generally refers to nucleic acid molecules generated by PCR using a given set of amplification primers.

As used herein, a “primer” defines an oligonucleotide which is capable of annealing to (hybridizing with) a target sequence, thereby creating a double stranded region which can serve as an initiation point for DNA synthesis under suitable conditions. The terminology “primer pair” refers herein to a pair of oligonucleotides which are selected to be used together in amplifying a selected nucleic acid sequence by one of a number of types of amplification processes, preferably PCR. The primers may be designed to bind to a complementary sequence under selected conditions.

The primers may be of any suitable length, depending on the particular assay format and the particular needs. In some embodiments, the primers may include at least 15 nucleotides in length, preferably between 15-25 nucleotides in length, more preferably between 18-25 nucleotides in length. The primers may be adapted to be especially suited to a chosen nucleic acid amplification system. The oligonucleotide primers may be designed by taking into consideration the melting point of hybridization thereof with their targeted sequence.

In some embodiments, the marker and control loci may be amplified from the same DNA sample (the digested sample) using pairs of reverse and forward primers to specifically amplify each locus. In some embodiments, the primers may be designed to amplify a locus along with 5′ and 3′ flanking sequences thereof.

In some embodiments, the 5′ flanking sequences may include between 1-60 bases immediately upstream of the locus. In additional embodiments, the 5′ flanking sequences may include between 1-35 bases immediately upstream of the locus.

In some embodiments, the 3′ flanking sequences may include between 1-60 bases immediately downstream of the locus. In additional embodiments, the 3′ flanking sequences may include between 20-60 bases immediately downstream of the locus.

In some embodiments, the primers may be designed to generate amplification products of between 30-150 bps in length when the locus is intact. In some particular embodiments, the primers may be designed to generate amplification products of between 80-150 bps in length.

In some embodiments, the methods of the present invention involve simultaneous amplification of more than one target sequence (at least one marker locus and a control locus) in the same reaction mixture, a process known as multiplex amplification or co-amplification. This process requires simultaneous use of multiple primer pairs. The primers may be designed such that they can work at the same annealing temperature during amplification. In some embodiments, primers with similar melting temperature (Tm) are used in the methods disclosed herein. A Tm variation of between about 3°−5° C. is considered acceptable for primers used in a pool.

In some embodiments, all marker and control loci may be amplified in a single reaction mixture. In other embodiments, for example due to technical limitation of a particular machine, the digested DNA sample may be divided into several aliquots, each of which is supplemented with primer pairs for amplification of one or more marker loci and the control locus. Thus, even if a DNA sample is divided into several aliquots, the control locus is amplified in each aliquot, and calculation of signal ratios is performed for the control locus and a marker locus that were amplified together, i.e., from the same aliquot.

In some embodiments, amplification of the genomic loci may be carried out using real-time PCR, also known as quantitative PCR (qPCR), in which simultaneous amplification and detection of the amplification products are performed.

In some embodiments, detection of the amplification products in real-time PCR may be achieved using polynucleotide probes, typically fluorescently-labeled polynucleotide probes.

As used herein, “polynucleotide probes” or “oligonucleotide probes” are interchangeable and refer to labeled polynucleotides which are complementary to specific sub-sequences within the nucleic acid sequences of loci of interest, for example, within the sequence of a marker locus or a control locus. In some embodiments, detection is achieved by using TaqMan assays based on combined reporter and quencher molecules (Roche Molecular Systems Inc.). In such assays, the polynucleotide probes have a fluorescent moiety (fluorophore) attached to their 5′ end and a quencher attached to the 3′ end. During PCR amplification, the polynucleotide probes selectively hybridize to their target sequences on the template, and as the polymerase replicates the template it also cleaves the polynucleotide probes due to the polymerase's 5′—nuclease activity. When the polynucleotide probes are intact, the close proximity between the quencher and the fluorescent moiety normally results in a low level of background fluorescence. When the polynucleotide probes are cleaved, the quencher is decoupled from the fluorescent moiety, resulting in an increase of intensity of fluorescence. The fluorescent signal correlates with the amount of amplification products, i.e., the signal increases as the amplification products accumulate.

As used herein, “selectively hybridize to” (as well as “selective hybridization,” “specifically hybridize to,” and “specific hybridization”) refers to the binding, duplexing, or hybridizing of a nucleic acid molecule (such as a primer or a probe) preferentially to a particular complementary nucleotide sequence under stringent conditions. The term “stringent conditions” refers to conditions under which a nucleic acid molecule will hybridize preferentially to its target sequence and to a lesser extent to, or not at all to, other non-target sequences. A “stringent hybridization” in the context of nucleic acid hybridization is sequence-dependent, and differs under different conditions, as known in the art.

Polynucleotide probes may vary in length. In some embodiments, the polynucleotide probes may include between 15-30 bases. In additional embodiments, the polynucleotide probes may include between 25-30 bases. In some embodiments, the polynucleotide probes may include between 20-30 bases, for example, 20 bases, 21 bases, 22 bases, 23 bases, 24 bases, 25 bases, 26 bases, 27 bases, 28 bases, 29 bases, 30 bases. Each possibility represents a separate embodiment of the present invention.

Polynucleotide probes may be designed to bind to either strand of the template. Additional considerations include the Tm of the polynucleotide probes, which should preferably be compatible to that of the primers. Computer software may be used for designing the primers and probes.

As noted above, the methods disclosed herein may involve simultaneous amplification of more than one target sequence (at least one marker locus and a control locus) in the same reaction mixture. In order to distinguish between multiple target sequences that are amplified in parallel, polynucleotide probes labeled with distinct fluorescent colors may be used. In some embodiments, the polynucleotide probes form fluorophore/quencher pairs as known in the art, and include, for example, FAM-TAMRA, FAM-BHQ1, Yakima Yellow-BHQ1, ATT0550-BHQ2 and ROX-BHQ2. In some embodiments, the dye combinations may be compatible to the real-time PCR thermocycler of choice.

In some embodiments, fluorescence may be monitored during each PCR cycle, providing an amplification plot showing the change of fluorescent signals from the probes as a function of cycle number.

In the context of real-time PCR, the following terminology is used:

“Quantification cycle” (“Cq”) refers to the cycle number in which fluorescence increases above a threshold, set automatically by software or manually by the user. In some embodiments, the threshold may be constant for all loci and may be set in advance, prior to carrying out the amplification and detection. In other embodiments, the threshold may be defined separately for each locus after the run, based on the maximum fluorescence level detected for this locus during the amplification cycles.

“Threshold” refers to a value of fluorescence used for Cq determination. In some embodiments, the threshold value may be a value above baseline fluorescence, and/or above background noise, and within the exponential growth phase of the amplification plot.

“Baseline” refers to the initial cycles of PCR where there is little to no change in fluorescence.

Computer software may be used to analyze amplification plots and determine baseline, threshold and Cq.

Following digestion with the at least one methylation-sensitive restriction enzyme, marker loci in which CG dinucleotide(s) in the enzyme's recognition site are methylated are amplified to a high level, because the DNA molecules are protected from cleavage. The result is relatively low Cq values because detectable amplification products are shown following a relatively small number of amplification cycles. Conversely, marker loci in which CG dinucleotide(s) in the enzyme's recognition site are unmethylated are cut more extensively during the digestion step, and thus result in higher Cq values in the amplification and quantification step (i.e., show detectable amplification products following a relatively large number of amplification cycles).

In alternative embodiments, amplification and detection of amplification products may be carried out by conventional PCR using fluorescently-labeled primers followed by capillary electrophoresis of amplification products. In some embodiments, following amplification the amplification products are separated by capillary electrophoresis and fluorescent signals are quantified. In some embodiments, an electropherogram plotting the change in fluorescent signals as a function of size (bp) or time from injection may be generated, wherein each peak in the electropherogram corresponds to the amplification product of a single locus. The peak's height (provided for example using “relative fluorescent units”, rFU) may represent the intensity of the signal from the amplified locus. Computer software may be used to detect peaks and calculate the fluorescence intensities (peak heights) of a set of loci whose amplification products were run on the capillary electrophoresis machine, and subsequently the ratios between the signal intensities.

For DNA samples digested with a methylation-sensitive restriction enzyme, marker loci in which CG dinucleotide(s) in the enzyme's recognition site are methylated produce a relatively strong signal (higher peak) in the electropherogram. Conversely, marker loci in which the CG dinucleotide(s) in the enzyme's recognition site are unmethylated produce a relatively weak signal (lower peak) in the electropherogram.

In some embodiments, the fluorescent labels of the primers include any one of fluorescein, FAM, lissamine, phycoerythrin, rhodamine, Cy2, Cy3, Cy3.5, Cy5, Cy5.5, Cy7, FluorX, JOE, HEX, NED, VIC and ROX.

C. Signal ratio

The term “ratio” or “signal ratio” as used herein refers to the ratio between the intensities of signals obtained from co-amplification of a pair of genomic loci in the same DNA sample (in the same reaction mixture), particularly co-amplification of a marker locus and a control locus. A signal ratio between a marker locus and a control locus of the present invention, obtained following methylation-sensitive enzymatic digestion and co-amplification, reflects the methylation ratio between these marker and control loci, and represents a “methylation value” according to the present invention.

The term “signal intensity” as used herein refers to a measure reflecting the amount of locus-specific amplification products corresponding to the initial amount of intact copies of the locus. However, the signal intensity may not indicate actual amounts of amplification products/intact loci, and may not involve calculation of any absolute amounts of amplification products/intact loci. Thus, for calculating ratios of amplicon signals, no standard curve or reference DNA may be needed since it is unnecessary to calculate actual DNA concentrations or DNA methylation level per se.

In some exemplary embodiments, amplification and detection of amplification products are carried out by real-time PCR, where the signal intensity of a specific locus may be represented by the Cq calculated for this locus. The signal ratio in this case may be represented by the following calculation:

2^{(Cq of control locus-Cq of marker locus)}.

In additional exemplary embodiments, detection of amplification products is carried out by capillary electrophoresis wherein the signal intensity of a specific locus is the number of relative fluorescence units (rFUs) of its corresponding peak. The signal ratio may be calculated by dividing the heights of peaks of each marker locus by the height of the peak of a control locus.

In some embodiments, calculating a ratio between signal intensities of the amplification products of a marker locus and a control locus in a DNA sample comprises: (i) determining the signal intensity of the amplification product of the marker locus; (ii) determining the signal intensity of the amplification product of the control locus; and (iii) calculating a ratio between the two signal intensities.

In some embodiments, calculating a ratio between signal intensities of the amplification products of a marker locus and a control locus in the DNA sample comprises determining the Cq for each locus, and calculating the difference between the Cq of the control locus and the Cq of the marker locus. In some embodiments, the calculating further comprises applying the following formula: 2\hat{ }(Cq of control locus—Cq of marker locus).

In some embodiments, calculating a signal ratio may be calculating a plurality of signal ratios, between each marker locus and a control locus.

In some embodiments, a plurality of genomic loci among the marker loci set forth in SEQ ID NOs: 1-6, 8-15 are amplified, and the methods of the present invention comprise calculating signal ratios between each of the marker loci set forth SEQ ID NOs: 1-6, 8-15 and a control locus, e.g., between the marker locus set forth in SEQ ID NO: 1 and the control locus, between the marker locus set forth in SEQ ID NO: 2 and the control locus, and so forth.

In some embodiments, computer software may be used for calculating a ratio between signal intensities of amplification products.

As noted above, a signal ratio between a marker locus and a control locus of the present invention, obtained following methylation-sensitive enzymatic digestion and co-amplification, reflects the methylation ratio between these marker and control loci, and represents a “methylation value” according to the present invention.

Methylation Analysis Using Methylation-Sensitive Enzymatic Digestion and High-Throughput Sequencing

“High throughput sequencing” (also termed “next generation sequencing”, abbreviated “NGS”) includes sequence determination using methods that determine many (typically thousands to billions) of nucleic acid sequences in parallel. High throughput sequencing generally involves three basic steps: library preparation, sequencing and data analysis. Examples of high throughput sequencing techniques include sequencing-by-synthesis and sequencing-by-ligation (employed, for example, by lllumina Inc., Life Technologies Inc., Roche), nanopore sequencing methods and electronic detection-based methods such as Ion Torrent™ technology (Life Technologies Inc.). High-throughput sequencing include whole genome high-throughput sequencing and target-specific high-throughput sequencing. Each possibility represents a separate embodiment of the present invention.

According to some embodiments of the present invention, following extraction the DNA is subjected to digestion with at least one methylation-sensitive restriction endonuclease as described herein, and subsequently a sequencing library is prepared.

In some embodiments, preparing a sequencing library comprises introducing adapter oligonucleotides, also termed “sequencing adapters”, to DNA fragments, and enriching DNA fragments corresponding to marker loci of interest and optionally one or more control loci.

Enrichment of genomic regions of interest may be carried out, for example, using locus-specific PCR, or using capture agents in a solution-phase or a solid-phase hybridization-based process.

The sequencing adapters are oligonucleotides at the 5′ and 3′ ends of each DNA fragment in a sequencing library. Sequencing adapters typically include platform-specific sequences for fragment recognition by a particular sequencer: for example, sequences that enable library fragments to bind to the flow cells of Illumina platforms. Each sequencing instrument typically employs a specific set of sequences for this purpose. The sequencing adapters may include sample indices, which are sequences that enable multiple samples to be sequenced together (i.e., multiplexed) on the same instrument flow cell or chip. Each sample index, typically 6-10 bases, is specific to a given sample library and is used for de-multiplexing during data analysis to assign individual sequence reads to the correct sample. Sequencing adapters may contain single or dual sample indexes depending on the number of libraries combined and the level of accuracy desired.

Sequencing adapters may be introduced into analyzed DNA fragments by ligation or via PCR. In some embodiments, a 2-step PCR is used, in order to enrich genomic regions of interest and introduce sequencing adapters to the enriched fragments. The first PCR is carried out using primers that contain locus-specific sequences and overhang sequences that introduce a first portion of the sequencing adapters, and the second PCR is carried out using primers that introduce a second portion of the sequencing adapters and optionally sample indices.

A sequencing library is subjected to sequencing to obtain multiple sequence reads. The sequence reads are analyzed using a computer software to determine a read count (copy number) for each locus of interest. The read count of a marker locus reflects the number of methylated copies of this locus that were present in the tested DNA sample (the methylated copies remain intact when the sample is digested with methylation-sensitive restriction endonucleases). Relative copy number may be calculated for a marker locus, for example, with respect to a control locus, as follows:

Relative copy number=Read count of marker locus/Read count of control locus A read count of a marker locus or alternatively a relative copy number of a marker locus represent “methylation values” according to the present invention.

In some embodiments, analysis of methylation values according to the present invention comprises:
(a) subjecting a DNA sample to digestion with at least one methylation-sensitive restriction endonuclease, thereby obtaining restriction endonuclease-treated DNA;
(b) generating a sequencing library from the restriction endonuclease-treated DNA, the sequencing library comprising DNA fragments corresponding to at least one marker locus as disclosed herein (preferably a plurality of marker loci as disclosed herein) and at least one control locus;
(c) subjecting the sequencing library to high-throughput sequencing and determining a copy number for each of the at least one marker locus and the control locus; and
(d) comparing a ratio between the copy numbers of each of said at least one marker locus and the control locus to at least one reference ratio, to assess the presence of cancer in a subject and/or indicate a tissue source of the cancer.

In some embodiments, generating a sequencing library comprises enriching DNA fragments corresponding to the at least one marker locus and the at least one control locus.

Assessing the Presence of Cancer and Indicating a Tissue Source

The methylation value(s) determined in a DNA sample for one or more of the pan-cancer marker loci disclosed herein, and/or for one or more of the tissue-specificity marker loci disclosed herein, constitute a methylation profile of the DNA sample.

As disclosed herein, the methylation value(s) are compared to at least one reference methylation values in order to assess the presence of cancer and/or predict the tissue source of cancer.

“Assessing cancer” or “assessing the presence of cancer” as used herein refer to determining the likelihood that a subject has cancer. The terms encompass determining whether a subject should be subjected to confirmatory cancer testing to confirm (or rule out) the presence of cancer, such as confirmatory blood tests, urine tests, cytology, imaging, endoscopy and/or biopsy. The terms further encompass aiding the diagnosis of cancer in a subject. The terms further encompass quantifying cancer-related changes in cell-free DNA samples which are indicative for the presence of cancer. Assessing the presence of cancer according to the present invention includes one or more of screening for cancer, assessing recurrence of cancer, assessing susceptibility or risk to cancer, assessing and/or monitoring response to treatment, assessing efficacy of treatment, assessing severity (stage) of cancer and assessing prognosis of cancer in a subject. Each possibility represents a separate embodiment of the present invention. It is to be understood that a negative result in the assays disclosed herein is still considered an assessment for the presence of cancer according to the present invention.

“Indicating the tissue source of cancer” or “providing an indication of the tissue source” as used herein refer to indicating a tissue source which is the most probable tissue source of the cancer out of a group of tissue sources. As described herein, the indication of the tissue source is based on a statistical analysis (a computerized analysis) that compares the methylation values of tissue-specificity marker loci determined in a tested DNA sample to reference values. In some embodiments, indicating a tissue source comprises generating a plurality of likelihood scores, for each tissue source that is being evaluated. The most probable tissue source is then selected based on the likelihood scores. In some embodiments, indicating a tissue source according to the present invention is used for determining the most appropriate follow-up test(s) for definitive diagnosis and/or monitoring of the cancer.

A “reference methylation value” as disclosed herein refers to a methylation value determined for a particular marker locus in DNA from a known source. For example, a “cancer reference methylation value” refers to a reference methylation value determined in cancer patients. A “non-cancer reference methylation value” (used herein interchangeably with “normal” or “healthy” methylation value) refers to a reference methylation value determined in healthy subjects with no cancer. A “healthy” or “normal” or “non-cancer” subject/individual is defined herein as an individual without detectable symptoms and/or pathological findings of cancer, as determined by conventional diagnostic methods. The term also encompasses an individual that did not develop cancer within a defined period of time in which the individual was followed-up for the development cancer.

A reference methylation value of a particular cancer tissue source (e.g., lung cancer reference methylation value) refers to a reference methylation value determined in cancer patients with the particular type of cancer (e.g., in lung cancer patients).

A “reference DNA sample” is a DNA sample from a known source. In some embodiments, a reference methylation value is a methylation value determined in a plurality of reference DNA samples. In addition, the methods of the present invention may be used for analyzing (e.g., measuring) methylation changes between DNA samples taken from a single subject at different time points, for example, taken at different stages of a disease, or taken before and after treatment of a disease. The methylation value(s) of the DNA sample taken at a first time point may be used as a reference for the methylation value(s) of a DNA sample taken at a second (later) time point.

In some particular embodiments, the reference methylation value is a reference ratio between signal intensities of a marker locus and a control locus following methylation-sensitive enzymatic digestion of the DNA sample and amplification, as described herein.

In some embodiments, a reference methylation value for a certain marker locus may be a single value. In some embodiments, the reference methylation value for a certain marker locus may be a statistic value, such as, a mean value determined from a large set of reference methylation values, e.g., mean of methylation values determined in a large group of cancer patients or mean of methylation values determined in a large group of healthy individuals.

In other embodiments, the reference methylation value for a certain marker locus may be a plurality of values, such as a distribution of methylation values determined for this marker locus in a large set of DNA samples from a known source. In some embodiments, the reference methylation value may be a reference scale.

A reference scale for a particular marker locus may include methylation values determined for this marker locus in a plurality of DNA samples from a known reference source. For example, a reference scale of reference cancer patients or a reference scale of reference healthy individuals. Alternatively, a reference scale for a particular marker locus may include methylation values from two types of sources of DNA, for example, from both healthy and cancer individuals. For example, a single scale combining reference methylation values from healthy individuals and cancer patients. Generally, when a single scale is used, the values are distributed such that the values from one source of DNA (e.g., the healthy individuals) are at one end of the scale, e.g., below a cutoff, while the values from another source of DNA (e.g., cancer patients) are at the other end of the scale, e.g., above the cutoff. In some embodiments, methylation value(s) calculated for a tested DNA sample from an unknown source may be compared against a reference scale of DNA from known source(s), and a score may be assigned to the calculated methylation value(s) based on the relative position of the calculated methylation value(s) within the scale.

In some embodiments, the methods disclosed herein comprise predetermination of reference methylation value(s). It is to be understood that reference methylation value(s) are determined using the same method that is used for determining methylation value(s) of a tested DNA sample.

A person of skill in the art would appreciate that the comparison of methylation value(s) calculated for a tested DNA sample to one or more corresponding reference values may be performed in a number of ways, using various statistical means.

In some embodiments, comparing a test methylation value calculated for a particular marker locus in a tested DNA sample to a reference value comprises comparing the test value against a single reference value. The single reference value may correspond, e.g., to a mean value obtained from reference methylation values of a large population of reference DNA samples. In other embodiments, comparing a test value to a reference value comprises comparing the test value against a distribution, or a scale, of a plurality of reference values. Statistical means may be employed in order to determine whether a methylation value calculated for a tested DNA sample matches a particular reference value, for example, a cancer reference value or a non-cancer reference value.

In some embodiments, cancer detection or assessment according to the present invention is based on analyzing whether methylation value(s) of a tested DNA sample are cancer value(s). In additional embodiments, cancer detection or assessment according to the present invention is based on analyzing whether methylation value(s) of a tested DNA sample are non-cancer value(s).

In some embodiments, determining or predicting the tissue source of cancer according to the present invention is based on analyzing whether methylation value(s) of a tested DNA sample match a first or second tissue source. In some embodiments, determining or predicting the tissue source of cancer according to the present invention is based on analyzing whether methylation value(s) of a tested DNA sample match lung cancer values or values of a different type of cancer, for example, lung cancer or liver cancer, lung cancer or colorectal cancer, lung cancer or breast cancer, lung cancer or a hematological cancer. Each possibility represents a separate embodiment of the present invention.

In some embodiments, the methods disclosed herein comprise comparing a methylation value calculated for a tested DNA sample to a reference methylation value of a particular source of DNA, to obtain a score reflecting the likelihood that the DNA is from this particular source. For example, comparing a methylation value calculated for a tested DNA sample to a cancer reference value, to obtain a score reflecting the likelihood that the tested DNA is cancer DNA. In some embodiments, the higher the score, the higher the likelihood that the DNA is from the particular source being evaluated. In some embodiments, a score is assigned based on the relative position of a calculated methylation value within a distribution of reference values.

In some embodiments, the methods disclosed herein comprise comparing a plurality of methylation values calculated for a plurality of marker loci to corresponding reference values. In some embodiments, a pattern of methylation values is analyzed using statistical means and computerized algorithm(s) to determine the source of DNA that the pattern represents. Exemplary algorithms include machine learning and pattern recognition algorithms.

In some exemplary embodiments, a methylation value calculated for a tested DNA sample may be compared against a scale of reference values generated from a large set of reference DNA samples. The scale may exhibit a threshold value, also termed herein a “cutoff” value, which differentiates between different source of DNA. Methylation values below the threshold are classified as a first source of DNA, while methylation values above the threshold are classified as a second source of DNA (e.g., normal vs. cancer, or lung cancer vs. a different type of cancer).

For analysis of a plurality of marker loci, the methylation value calculated for each marker locus may be given a score based on its relative position within a reference scale for this marker locus, and subsequently the individual scores (for each marker locus) may be combined to give a single score. In some embodiments, the individual scores may be summed to give a single score. In other embodiments, the individual scores may be averaged to give a single score.

In some embodiments, a combined score may be used for assessing whether cancer is present or absent in a tested subject, where a score above a predefined threshold is indicative of the presence of cancer, and a score below the predefined threshold is indicative of the absence of cancer.

In additional embodiments, a combined score may be used for determining or predicting whether the tissue source of the cancer is a first or second tissue source, where a score above a predefined threshold is indicative of the first tissue source, and a score below the predefined threshold is indicative of the second tissue source.

In some embodiments, analysis of a plurality of marker loci comprises calculating for each marker locus the probability that its methylation value represents a cancer methylation value, based on comparison to a corresponding cancer reference value and/or a normal reference value. A score may be assigned for each marker locus, and subsequently the individual scores may be combined (e.g., summed or averaged) to give a combined score. The combined score may be used for assessing whether the subject is positive or negative for cancer, wherein a combined score above a predefined threshold is indicative of cancer.

In some embodiments, analysis of a plurality of marker loci comprises calculating for each marker locus the probability that its methylation value represents a methylation value of a particular tissue source, based on comparison to reference values of two tissue sources. A score may be assigned for each marker locus, and subsequently the individual scores may be combined (e.g., summed or averaged) to give a combined score. The combined score may be used for determining or predicting the tissue source of cancer in a subject, wherein a combined score above a predefined threshold is indicative of a first tissue source and a combined score below the predefined threshold is indicative of a second tissue source.

In some embodiments, the methods according to the present invention comprise providing one or more threshold scores, that differentiate between sources of DNA. The threshold values are typically statistically significant values.

Statistical significance is often determined by comparing two or more populations, and determining a confidence interval (CI) and/or a p value. In some embodiments, the statistically significant values refer to confidence intervals (CI) of 90%, 95%, 97.5%, 98%, 99%, 99.5%, 99.9% and 99.99%, while preferred p values are less than 0.1, 0.05, 0.025, 0.02, 0.01, 0.005, 0.001 or less than 0.0001. Each possibility represents a separate embodiment of the present invention. According to some embodiments, the p value of a threshold score is at most 0.05.

In some embodiments, the overall sensitivity of cancer assessment using the methods disclosed herein is at least 60%. The “sensitivity” of cancer assessment according to the present invention is the percentage of diseased individuals who test positive (percent of “true positives”). Accordingly, diseased individuals not detected by the cancer assessment methods disclosed herein are “false negatives”. Subjects who are not diseased and who test negative are termed “true negatives”. The “specificity” of cancer assessment according to the present invention is one (1) minus the false positive rate, where the “false positive” rate is defined as the proportion of those without the disease who test positive. While a particular assessment method may not provide a definitive diagnosis of a condition, it suffices if the method provides a positive indication that aids in diagnosis. In some embodiments, the overall specificity of the cancer assessment methods disclosed herein may be at least 90%.

In some embodiments, the methods according to the present invention comprise preparing a report (in paper or electronic form) based on the methylation values. The report may be communicated to the subject and/or to a healthcare provider of the subject.

In some embodiments, the methods according to the present invention comprise referring the subject to follow-up cancer surveillance and testing.

Systems and Kits

In some embodiments, there is provided herein systems for assessing the presence of cancer in a subject based on methylation analysis of cfDNA of the subject, and optionally also providing an indication of the tissue source of the cancer based on methylation analysis of cfDNA of the subject. In additional embodiments, there is provided herein kits for assessing the presence of cancer in a subject based on methylation analysis of cfDNA of the subject, and optionally also indicating the tissue source of the cancer based on methylation analysis of cfDNA of the subject. In some embodiments, there is provided herein systems for indicating the tissue source of a cancer in a subject based on methylation analysis of cfDNA of the subject. In additional embodiments, there is provided herein kits for indicating the tissue source of a cancer in a subject based on methylation analysis of cfDNA of the subject.

In some embodiments, the systems and kits are for assessing the presence of cancer, and/or indicating the tissue source of the cancer, according to the methods disclosed herein.

In some embodiments, there is provided herein kits and systems for profiling methylation of a cell-free DNA sample of a subject according to the methods disclosed herein.

Systems according to the present invention comprise computer processor(s) for performing the assays and/or processing the results e.g., for performing the calculations. In some embodiments, computer-implemented methods are provided herein.

In some embodiments, a system according to the present invention comprises:

a cell-free DNA sample of a human subject;

components for carrying out a methylation assay on at least one marker locus hypermethylated in cancer DNA compared to non-cancer DNA selected from the group consisting of SEQ ID NO: 1, SEQ ID NO: 2 and SEQ ID NO: 3, optionally further comprising components for carrying out a methylation assay on at least one marker locus hypermethylated in cancer DNA compared to non-cancer DNA selected from the group consisting of SEQ ID NO: 4, SEQ ID NO: 5 and SEQ ID NO: 6; and

computer software stored on a non-transitory computer readable medium, the computer software directs a computer processor to determine a methylation value for the at least one marker locus based on the methylation assay, and compare the methylation value of each of said at least one marker locus to at least one reference methylation value selected from a cancer methylation value and a non-cancer methylation value, to assess the presence of cancer in the subject according to the methods disclosed herein.

In additional embodiments, a system according to the present invention comprises:

a cell-free DNA sample of a human subject;

components for carrying out a methylation assay on at least one tissue-specificity marker locus selected from: (A) one or more tissue specificity marker locus distinguishing between lung cancer and colorectal cancer selected from SEQ ID NO: 5 and SEQ ID NO: 8; (B) one or more tissue specificity marker locus distinguishing between lung cancer and liver cancer selected from SEQ ID NO: 9 and SEQ ID NO: 10; (C) one or more tissue specificity marker locus distinguishing between lung cancer and breast cancer selected from SEQ ID NO: 11 and SEQ ID NO: 12; and (D) one or more tissue specificity marker locus distinguishing between lung cancer and hematological cancer selected from SEQ ID NO: 13, SEQ ID NO: 14 and SEQ ID NO: 15; and

computer software stored on a non-transitory computer readable medium, the computer software directs a computer processor to provide an indication of the tissue source of the cancer based on the methylation assay according to the methods disclosed herein.

Components for carrying out a methylation assay encompass biochemical components (e.g., enzymes, primers, probes, nucleotides), chemical components (e.g., buffers, reagents), and technical components (e.g., a PCR system, such as a real-time PCR system, and equipment such as tubes, vials, plates, pipettes).

In some particular embodiments, a system according to the present invention comprises:

a cell-free DNA sample of a human subject;

at least one methylation-sensitive restriction endonuclease recognizing a sequence within at least one marker locus that is hypermethylated in cancer DNA compared to non-cancer DNA selected from the group consisting of SEQ ID NO: 1, SEQ ID NO: 2 and SEQ ID NO: 3, optionally wherein the at least one marker locus further comprises one or more of SEQ ID NO: 4, SEQ ID NO: 5 and SEQ ID NO: 6;

a plurality of primer pairs for co-amplification of the at least one marker locus and a control locus following digestion with the at least one methylation-sensitive restriction endonuclease;

components for detecting amplification products of the at least one marker locus and the control locus; and

computer software stored on a non-transitory computer readable medium, the computer software directs a computer processor to determine a signal intensity for each of the amplification products of the at least one marker locus and the control locus, and compare a ratio between the signal intensities of the amplification products of each of said at least one marker locus and the control locus to at least one reference ratio selected from cancer reference ratio and non-cancer reference ratio, to assess whether cancer is present in the subject according to the methods disclosed herein.

In additional particular embodiments, a system according to the present invention comprises:

a cell-free DNA sample of a human subject;

at least one methylation-sensitive restriction endonuclease recognizing a sequence within at least one tissue-specificity marker locus selected from: (A) one or more tissue specificity marker locus distinguishing between lung cancer and colorectal cancer selected from SEQ ID NO: 5 and SEQ ID NO: 8; (B) one or more tissue specificity marker locus distinguishing between lung cancer and liver cancer selected from SEQ ID NO: 9 and SEQ ID NO: 10; (C) one or more tissue specificity marker locus distinguishing between lung cancer and breast cancer selected from SEQ ID NO: 11 and SEQ ID NO: 12; and (D) one or more tissue specificity marker locus distinguishing between lung cancer and hematological cancer selected from SEQ ID NO: 13, SEQ ID NO: 14 and SEQ ID NO: 15;

a plurality of primer pairs for co-amplification of the at least one marker locus and a control locus following digestion with the at least one methylation-sensitive restriction endonuclease;

components for detecting amplification products of the at least one marker locus and the control locus; and

computer software stored on a non-transitory computer readable medium, the computer software directs a computer processor to determine a signal intensity for each of the amplification products of the at least one marker locus and the control locus, and compare a ratio between the signal intensities of the amplification products of each of said at least one marker locus and the control locus to at least one reference ratio to provide an indication of the tissue source of the cancer according to the methods disclosed herein.

Components for detecting amplification products of the at least one marker locus and the control locus encompass for example fluorescent labels, e.g., in the form of fluorescent primers or fluorescent probes capable of specific hybridization to the amplification products.

In some embodiments, a system according to the present invention prepares and communicates a report to the subject and/or to a healthcare provider of the subject based on the methylation values.

In some embodiments, there is provided herein a system for assessing the presence of cancer in a subject according to the methods disclosed herein, the system comprising a computer software stored on a non-transitory computer readable medium comprising instructions that when executed configure or direct a computer processor to perform the following steps:
(i) receiving a methylation value determined in a cell-free DNA sample of the subject for at least one marker locus that is hypermethylated in cancer DNA compared to non-cancer DNA selected from the group consisting of SEQ ID NO: 1, SEQ ID NO: 2 and SEQ ID NO: 3, optionally wherein the at least one marker locus further comprises one or more of SEQ ID NO: 4, SEQ ID NO: 5 and SEQ ID NO: 6; and
(ii) comparing the methylation value of each of said at least one marker locus to at least one reference methylation value selected from a cancer methylation value and a non-cancer methylation value, and based on the comparison, outputting an assessment whether cancer is present or absent in the subject

In some embodiments, the computer software further comprises instructions that when executed configure or direct the computer processor to determine the methylation value of each of said at least one marker locus based on data from a methylation assay.

In some embodiments, there is provided a system for providing an indication of a cancer tissue source in a subject according to the methods disclosed herein, the system comprising a computer software stored on a non-transitory computer readable medium comprising instructions that when executed configure or direct a computer processor to perform at least one of the following steps:
(i) receiving a methylation value determined in a cell-free DNA sample of the subject for at least one marker locus selected from SEQ ID NO: 5 and SEQ ID NO: 8, comparing the methylation value of each of said at least one marker locus to corresponding lung cancer and colorectal cancer reference values, and based on the comparison, outputting whether the methylation value represents a lung cancer methylation value or a colorectal cancer methylation value;
(ii) receiving a methylation value determined in a cell-free DNA sample of the subject for at least one marker locus selected from SEQ ID NO: 9 and SEQ ID NO: 10, comparing the methylation value of each of said at least one marker locus to corresponding lung cancer and liver cancer reference values, and based on the comparison, outputting whether the methylation value represents a lung cancer methylation value or a liver cancer methylation value;
(iii) receiving a methylation value determined in a cell-free DNA sample of the subject for at least one marker locus selected from SEQ ID NO: 11 and SEQ ID NO: 12, comparing the methylation value of each of said at least one marker locus to corresponding lung cancer and breast cancer reference values, and based on the comparison, outputting whether the methylation value represents a lung cancer methylation value or a breast cancer methylation value; and
(iv) receiving a methylation value determined in a cell-free DNA sample of the subject for at least one marker locus selected from SEQ ID NO: 13, SEQ ID NO: 14 and SEQ ID NO: 15, comparing the methylation value of each of said at least one marker locus to corresponding lung cancer and hematological cancer reference values, and based on the comparison, outputting whether the methylation value represents a lung cancer methylation value or a hematological cancer methylation value.

In some embodiments, the computer software further comprises instructions that when executed configure or direct the computer processor to determine the methylation value of each of said at least one marker locus based on data from a methylation assay.

In some embodiments, a computer software according to the present invention receives as an input raw data of a real-time PCR run. In some embodiments, the computer software directs a computer processor to analyze the real-time PCR data to determine methylation values, e.g., methylation ratios, as disclosed herein.

The computer software includes processor-executable instructions that are stored on a non-transitory computer readable medium. The computer software may also include stored data. The computer readable medium is a tangible computer readable medium, such as a compact disc (CD), magnetic storage, optical storage, random access memory (RAM), read only memory (ROM), or any other tangible medium.

It is understood that the computer-related methods, steps, processes described herein are implemented using software stored on non-volatile or non-transitory computer readable instructions that when executed configure or direct a computer processor or computer to perform the instructions.

Each of the system, server, computing device, and computer described in this application can be implemented on one or more computer systems and be configured to communicate over a network. They all may also be implemented on one single computer system. In one embodiment, the computer system includes a bus or other communication mechanism for communicating information, and a hardware processor coupled with bus for processing information.

The computer system also includes a main memory, such as a random-access memory (RAM) or other dynamic storage device, coupled to bus for storing information and instructions to be executed by processor. Main memory also may be used for storing temporary variables or other intermediate information during execution of instructions to be executed by processor. Such instructions, when stored in non-transitory storage media accessible to processor, render computer system into a special-purpose machine that is customized to perform the operations specified in the instructions.

The computer system further includes a read only memory (ROM) or other static storage device coupled to bus for storing static information and instructions for processor. A storage device, such as a magnetic disk or optical disk, is provided and coupled to bus for storing information and instructions.

The computer system may be coupled via bus to a display, for displaying information to a computer user.

An input device, including alphanumeric and other keys, is coupled to bus for communicating information and command selections to processor. Another type of user input device is cursor control, such as a mouse, a trackball, or cursor direction keys for communicating direction information and command selections to processor and for controlling cursor movement on display.

According to one embodiment, the techniques herein are performed by the computer system in response to the processor executing one or more sequences of one or more instructions contained in main memory. Such instructions may be read into main memory from another storage medium, such as storage device. Execution of the sequences of instructions contained in main memory causes the processor to perform the process steps described herein. In alternative embodiments, hard-wired circuitry may be used in place of or in combination with software instructions.

The term storage media as used herein refers to any non-transitory media that store data and/or instructions that cause a machine to operation in a specific fashion. Common forms of storage media include, for example, a floppy disk, a flexible disk, hard disk, solid state drive, magnetic tape, or any other magnetic data storage medium, a CD-ROM, any other optical data storage medium, any physical medium with patterns of holes, a RAM, a PROM, and EPROM, a FLASH-EPROM, NVRAM, any other memory chip or cartridge.

Storage media is distinct from but may be used in conjunction with transmission media. Transmission media participates in transferring information between storage media. For example, transmission media includes coaxial cables, copper wire and fiber optics, including the wires that comprise bus.

In some embodiments, a kit according to the present invention comprises components for carrying out a methylation assay on at least one marker locus as described herein in a cell-free DNA sample of a subject.

In some embodiments, a kit according to the present invention comprises:

at least one methylation-sensitive restriction endonuclease recognizing a sequence within at least one marker locus that is hypermethylated in cancer DNA compared to non-cancer DNA selected from the group consisting of SEQ ID NO: 1, SEQ ID NO: 2 and SEQ ID NO: 3, optionally wherein the at least one marker locus further comprises one or more of SEQ ID NO: 4, SEQ ID NO: 5 and SEQ ID NO: 6;

a plurality of primer pairs for co-amplification of the at least one marker locus and a control locus following digestion with the at least one methylation-sensitive restriction endonuclease; and

components for detecting amplification products of the at least one marker locus and the control locus as disclosed herein.

In some embodiments, the kit further comprises instruction manual for carrying out the assessment of the presence of cancer as disclosed herein.

In additional embodiments, a kit according to the present invention comprises:

at least one methylation-sensitive restriction endonuclease recognizing a sequence within at least one tissue-specificity marker locus selected from: (A) one or more tissue specificity marker locus distinguishing between lung cancer and colorectal cancer selected from SEQ ID NO: 5 and SEQ ID NO: 8; (B) one or more tissue specificity marker locus distinguishing between lung cancer and liver cancer selected from SEQ ID NO: 9 and SEQ ID NO: 10; (C) one or more tissue specificity marker locus distinguishing between lung cancer and breast cancer selected from SEQ ID NO: 11 and SEQ ID NO: 12; and (D) one or more tissue specificity marker locus distinguishing between lung cancer and hematological cancer selected from SEQ ID NO: 13, SEQ ID NO: 14 and SEQ ID NO: 15;
a plurality of primer pairs for co-amplification of the at least one marker locus and a control locus following digestion with the at least one methylation-sensitive restriction endonuclease; and

components for detecting amplification products of the at least one marker locus and the control locus as disclosed herein.

In some embodiments, the kit further comprises instruction manual for indicating a cancer tissue source as disclosed herein. In some embodiments, the instruction manual is an electronic instruction manual. In some embodiments, the instruction manual includes instructions for performing the method steps described herein. In some embodiments, the instruction manual provides one or more threshold score.

In some embodiments, a kit according to the present invention comprises a non-transitory computer readable medium storing a computer software comprising instructions that when executed configure or direct a computer processor to perform the method steps described herein.

In some embodiments, a kit or a system according to the present invention comprises a single methylation-sensitive restriction endonuclease. In some embodiments, a kit or a system according to the present invention comprises a plurality of methylation-sensitive restriction endonucleases, e.g., two methylation-sensitive restriction endonucleases. In some embodiments, the plurality of methylation-sensitive restriction endonucleases comprises Hinp1I. In additional embodiments, the plurality of methylation-sensitive restriction endonucleases comprises HhaI. In additional embodiments, the plurality of methylation-sensitive restriction endonucleases comprises AciI. In some particular embodiments, the kit comprises Hinp1I and AciI.

In some embodiments, a kit or a system according to the present invention comprises: at least one methylation-sensitive restriction endonucleases (e.g. Hinp1I and AciI); primer pairs complementary to at least one marker locus and at least one control locus as described herein; and fluorescent oligonucleotide probes complementary to the at least one marker locus and at least one control locus.

Exemplary primers for amplifying genomic loci comprising the sequences set forth as SEQ ID NOs: 1-15 are set forth as SEQ ID NOs: 16-195.

More particularly, exemplary forward primers for amplifying a genomic locus comprising SEQ ID NO: 1 are set forth as SEQ ID NOs: 16-21. Exemplary forward primers for amplifying a genomic locus comprising SEQ ID NO: 2 are set forth as SEQ ID NOs: 22-27. Exemplary forward primers for amplifying a genomic locus comprising SEQ ID NO: 3 are set forth as SEQ ID NOs: 28-33. Exemplary forward primers for amplifying a genomic locus comprising SEQ ID NO: 4 are set forth as SEQ ID NOs: 34-39. Exemplary forward primers for amplifying a genomic locus comprising SEQ ID NO: 5 are set forth as SEQ ID NOs: 40-45. Exemplary forward primers for amplifying a genomic locus comprising SEQ ID NO: 6 are set forth as SEQ ID NOs: 46-51. Exemplary forward primers for amplifying a genomic locus comprising SEQ ID NO: 7 are set forth as SEQ ID NOs: 52-57. Exemplary forward primers for amplifying a genomic locus comprising SEQ ID NO: 8 are set forth as SEQ ID NOs: 58-63. Exemplary forward primers for amplifying a genomic locus comprising SEQ ID NO: 9 are set forth as SEQ ID NOs: 64-69. Exemplary forward primers for amplifying a genomic locus comprising SEQ ID NO: 10 are set forth as SEQ ID NOs: 70-75. Exemplary forward primers for amplifying a genomic locus comprising SEQ ID NO: 11 are set forth as SEQ ID NOs: 76-81. Exemplary forward primers for amplifying a genomic locus comprising SEQ ID NO: 12 are set forth as SEQ ID NOs: 82-87. Exemplary forward primers for amplifying a genomic locus comprising SEQ ID NO: 13 are set forth as SEQ ID NOs: 88-93. Exemplary forward primers for amplifying a genomic locus comprising SEQ ID NO: 14 are set forth as SEQ ID NOs: 94-99. Exemplary forward primers for amplifying a genomic locus comprising SEQ ID NO: 15 are set forth as SEQ ID NOs: 100-105.

Exemplary reverse primers for amplifying a genomic locus comprising SEQ ID NO: 1 are set forth as SEQ ID NOs: 106-111. Exemplary reverse primers for amplifying a genomic locus comprising SEQ ID NO: 2 are set forth as SEQ ID NOs: 112-117. Exemplary reverse primers for amplifying a genomic locus comprising SEQ ID NO: 3 are set forth as SEQ ID NOs: 118-123. Exemplary reverse primers for amplifying a genomic locus comprising SEQ ID NO: 4 are set forth as SEQ ID NOs: 124-129. Exemplary reverse primers for amplifying a genomic locus comprising SEQ ID NO: 5 are set forth as SEQ ID NOs: 130-135. Exemplary reverse primers for amplifying a genomic locus comprising SEQ ID NO: 6 are set forth as SEQ ID NOs: 136-141. Exemplary reverse primers for amplifying a genomic locus comprising SEQ ID NO: 7 are set forth as SEQ ID NOs: 142-147. Exemplary reverse primers for amplifying a genomic locus comprising SEQ ID NO: 8 are set forth as SEQ ID NOs: 148-153. Exemplary reverse primers for amplifying a genomic locus comprising SEQ ID NO: 9 are set forth as SEQ ID NOs: 154-159. Exemplary reverse primers for amplifying a genomic locus comprising SEQ ID NO: 10 are set forth as SEQ ID NOs: 160-165. Exemplary reverse primers for amplifying a genomic locus comprising SEQ ID NO: 11 are set forth as SEQ ID NOs: 166-171. Exemplary reverse primers for amplifying a genomic locus comprising SEQ ID NO: 12 are set forth as SEQ ID NOs: 172-177. Exemplary reverse primers for amplifying a genomic locus comprising SEQ ID NO: 13 are set forth as SEQ ID NOs: 178-183. Exemplary reverse primers for amplifying a genomic locus comprising SEQ ID NO: 14 are set forth as SEQ ID NOs: 184-189. Exemplary reverse primers for amplifying a genomic locus comprising SEQ ID NO: 15 are set forth as SEQ ID NOs: 190-195.

In some embodiments, each nucleotide primer pair included in a kit or a system according to the present invention is designed to selectively amplify a fragment of the genome comprising a marker locus selected from SEQ ID NOs: 1-6, 8-15 and optionally a fragment of the genome comprising a control locus as set forth in SEQ ID NO: 7.

In some embodiments, a kit or a system according to the present invention comprises primer pairs for selectively amplifying the combination of marker loci described herein.

In some embodiments, a kit or a system according to the present invention includes oligonucleotide probes for detecting amplification products of the genomic loci amplified using the primer pairs. Each oligonucleotide probe is complementary to a sub-sequence within a genomic locus and capable of hybridizing thereto. In some embodiments, the oligonucleotide probes are fluorescently-labeled.

In some embodiments, there is provided herein an oligonucleotide probe for quantitative PCR, capable of hybridizing to an amplicon comprising a marker locus hypermethylated in cancer DNA compared to non-cancer DNA selected from the group consisting of SEQ ID NO: 1, SEQ ID NO: 2 and SEQ ID NO: 3. Each locus possibility represents a separate embodiment of the present invention.

In some embodiments, there is provided herein an oligonucleotide probe for quantitative PCR, capable of hybridizing to an amplicon comprising a tissue-specificity marker locus selected from the group consisting of SEQ ID NO: 8-15. Each locus possibility represents a separate embodiment of the present invention.

The term “amplicon” refers to a nucleic acid generated by a PCR amplification reaction of a target region of interest. An amplicon is typically up to 150 bps in length.

In some embodiments, there is provided herein a primer for producing an amplicon comprising a marker locus hypermethylated in cancer DNA compared to non-cancer DNA selected from the group consisting of SEQ ID NO: 1, SEQ ID NO: 2 and SEQ ID NO: 3. Each locus possibility represents a separate embodiment of the present invention.

In some embodiments, there is provided herein a primer for producing an amplicon comprising a tissue-specificity marker locus selected from the group consisting of SEQ ID NOs: 8-15. Each locus possibility represents a separate embodiment of the present invention.

In some embodiments, the primer is selected from the group consisting of SEQ ID NOs: 16-195.

In some embodiments, there is provided herein a primer pair capable of producing an amplicon comprising a marker locus hypermethylated in cancer DNA compared to non-cancer DNA selected from the group consisting of SEQ ID NO: 1, SEQ ID NO: 2 and SEQ ID NO: 3. Each locus possibility represents a separate embodiment of the present invention.

In some embodiments, there is provided herein a primer pair capable of producing an amplicon comprising a tissue-specificity marker locus selected from the group consisting of SEQ ID NOs: 8-15. Each locus possibility represents a separate embodiment of the present invention.

In some embodiments, the primer pair comprises a forward primer selected from the group consisting of SEQ ID NOs: 16-105 and a corresponding reverse primer selected from the group consisting of SEQ ID NOs: 106-195.

In some embodiments, there is provided herein an oligonucleotide combination for quantitative PCR, comprising: a primer pair capable of producing an amplicon comprising a marker locus hypermethylated in cancer DNA compared to non-cancer DNA selected from the group consisting of SEQ ID NO: 1, SEQ ID NO: 2 and SEQ ID NO: 3; and a quantitative PCR oligonucleotide probe, capable of hybridizing to the amplicon.

In some embodiments, there is provided herein an oligonucleotide combination for quantitative PCR, comprising: a primer pair capable of producing an amplicon comprising a tissue-specificity marker locus selected from the group consisting of SEQ ID NOs: 8-15; and a quantitative PCR oligonucleotide probe, capable of hybridizing to the amplicon.

### Methods of Treatment

The methods of the present invention further provide methods of treating or monitoring cancer comprising identifying a subject as being a cancer patient (e.g., breast, lung, colon cancer) by determining a methylation value as described herein and any one of the methods described herein, and administering an appropriate therapy or monitoring activity.

The terms “treatment,” “treat”, “treating,” and the like, are meant to include slowing or reversing the progression of a disease. These terms also include alleviating, ameliorating, attenuating, eliminating, or reducing one or more symptoms of a disease.

The term “administering” or “administration of” a substance, a compound or an agent to a subject can be carried out using one of a variety of methods known to those skilled in the art. For example, a compound or an agent can be administered enterally or parenterally.

According to an aspect, the present invention provides a method of care for a subject with cancer or for managing cancer or for treating cancer in a subject in need thereof, the method comprising:
(a) determining in a cell-free DNA (cfDNA) sample of the subject a methylation value for at least one marker locus hypermethylated in cancer DNA compared to non-cancer DNA selected from the group consisting of SEQ ID NO: 1, SEQ ID NO: 2 and SEQ ID NO: 3, optionally further determining a methylation value for at least one marker locus hypermethylated in cancer DNA compared to non-cancer DNA selected from the group consisting of SEQ ID NO: 4, SEQ ID NO: 5 and SEQ ID NO: 6;
(b) determining a positive assessment for the presence of cancer in the subject based on a comparison of the methylation value of each of said at least one marker locus to at least one reference methylation value selected from cancer reference methylation value and non-cancer reference methylation value; and
(c) administering to the subject active cancer surveillance and follow-up testing to identify the tissue source of the cancer, determine stage of the cancer and optionally monitor the progression of the cancer, wherein the cancer surveillance and follow-up testing comprises one or more of blood tests, urine tests, cytology, imaging, endoscopy and biopsy.

In some embodiments, step (c) comprises administering whole-body PET-CT scan to a subject with a positive assessment for the presence of cancer.

In some embodiments, the method further comprising a step of providing an indication of the tissue source of the cancer by analyzing the methylation of one or more tissue specificity marker locus distinguishing between lung cancer and colorectal cancer selected from SEQ ID NO: 5 and SEQ ID NO: 8. In some embodiments, the method further comprising a step of providing an indication of the tissue source of the cancer by analyzing the methylation of one or more tissue specificity marker locus distinguishing between lung cancer and liver cancer selected from SEQ ID NO: 9 and SEQ ID NO: 10. In some embodiments, the method further comprising a step of providing an indication of the tissue source of the cancer by analyzing the methylation of one or more tissue specificity marker locus distinguishing between lung cancer and breast cancer selected from SEQ ID NO: 11 and SEQ ID NO: 12. In some embodiments, the method further comprising a step of providing an indication of the tissue source of the cancer by analyzing the methylation of one or more tissue specificity marker locus distinguishing between lung cancer and hematological cancer selected from SEQ ID NO: 13, SEQ ID NO: 14 and SEQ ID NO: 15.

In some embodiments, the method further comprising a step of: providing an indication of the tissue source of the cancer by analyzing the methylation of at least one tissue-specificity marker locus selected from: (A) one or more tissue specificity marker locus distinguishing between lung cancer and colorectal cancer selected from SEQ ID NO: 5 and SEQ ID NO: 8; (B) one or more tissue specificity marker locus distinguishing between lung cancer and liver cancer selected from SEQ ID NO: 9 and SEQ ID NO: 10; (C) one or more tissue specificity marker locus distinguishing between lung cancer and breast cancer selected from SEQ ID NO: 11 and SEQ ID NO: 12; and (D) one or more tissue specificity marker locus distinguishing between lung cancer and hematological cancer selected from SEQ ID NO: 13, SEQ ID NO: 14 and SEQ ID NO: 15.

According to a further aspect, the present invention provides a method for treating or managing or take care of lung cancer in a subject in need thereof, the method comprising:
(a) determining in a cell-free DNA (cfDNA) sample of the subject a methylation value for at least one marker locus hypermethylated in cancer DNA compared to non-cancer DNA selected from the group consisting of SEQ ID NO: 1, SEQ ID NO: 2 and SEQ ID NO: 3, optionally further determining a methylation value for at least one marker locus hypermethylated in cancer DNA compared to non-cancer DNA selected from the group consisting of SEQ ID NO: 4, SEQ ID NO: 5 and SEQ ID NO: 6;
(b) determining a positive indication for the presence of cancer in the subject based on a comparison of the methylation value of each of said at least one marker locus to at least one reference methylation value selected from cancer reference methylation value and non-cancer reference methylation value;
(c) quantifying an increased risk for a presence of lung cancer in the subject by analyzing the methylation of at least one tissue-specificity marker locus selected from: (A) one or more tissue specificity marker locus distinguishing between lung cancer and colorectal cancer selected from SEQ ID NO: 5 and SEQ ID NO: 8; (B) one or more tissue specificity marker locus distinguishing between lung cancer and liver cancer selected from SEQ ID NO: 9 and SEQ ID NO: 10; (C) one or more tissue specificity marker locus distinguishing between lung cancer and breast cancer selected from SEQ ID NO: 11 and SEQ ID NO: 12; and (D) one or more tissue specificity marker locus distinguishing between lung cancer and hematological cancer selected from SEQ ID NO: 13, SEQ ID NO: 14 and SEQ ID NO: 15; and
(d) administering a computerized tomography (CT) scan to the subject with a quantified increased risk for the presence lung cancer.

According to a yet another aspect, the present invention provides a method for treating lung, colon, breast, bladder, liver, or renal cell cancer in a patient, comprising:
(i) identifying the patient as being a lung, colon, breast, bladder, liver, or renal cell cancer patient by any of the methods described herein;
(ii) determining a mutational status of one or more genes selected from the group of KRAS, EGFR, ALK, ROS1, BRAF, RET, MET and NTRK in a tumor DNA sample from the patient; and
(iii) administering a therapy targeted to the mutational status of the patient's tumor DNA

According to a yet another aspect, the present invention provides a method for treating occult or stage 0 lung cancer in a patient, comprising:
(i) identifying the patient as being a lung cancer patient by any one of the methods described herein; and
(ii) administering active lung cancer surveillance to said patient, wherein the active surveillance comprises one or more of a pulmonary imaging study, a sputum cytology study, a bronchoalveolar lavage cytology study, a bronchothoracic biopsy, and a transthoracic biopsy.

According to a yet another aspect, the present invention provides a method for performing computerized tomography (CT) on a patient, comprising:
(i) identifying the patient as being a lung cancer patient by any one of the methods described herein; and
(ii) performing a low-dose computed tomography (LDCT) scan on the chest of the patient.

According to a yet another aspect, the present invention provides a method for treating lung or breast cancer in a patient, comprising:
(i) identifying the patient as being a lung, breast, or colon cancer patient by any one of the methods described herein;
(ii) determining a PD-L1 status and optionally a mutational burden status in a tumor sample from the patient; and
(iii) assigning immunotherapy targeted to the PD-L1 status and optionally the mutational burden status of the tumor sample.

According to a yet another aspect, the present invention provides a method for treating a breast cancer patient, comprising:
(i) identifying the patient as being a breast cancer patient any one of the methods described herein;
(ii) determining an ER, a HER2 and a BRCA1/2 status in a tissue sample from the patient; and
(iii) administering a therapy targeted to the ER, HER2 and BRCA1/2 status of the patient.

According to a yet another aspect, the present invention provides a method for treating a colon cancer patient, comprising:
(i) identifying the patient as being a colon cancer patient by any one of the methods described herein;
(ii) determining a mismatch repair (MMR) or microsatellite instability (MSI) status in a tumor sample from the patient; and
(iii) administering a therapy targeted to the MMR or MSI status of the patient.

A number of targeted therapies are known in the art for carious cancer types. By way of example only, targeted therapies for non-small cell lung cancer include, but are not limited to, drugs that target angiogenesis (e.g., Bevacizumab, Ramucirumab); drugs that target cells with KRAS gene changes (e.g., Sotorasib); drugs that target cells with EGFR gene changes (e.g., Erlotinib, Afatinib, Gefitinib, Osimertinib, Dacomitinib, Amivantamab, Mobocertinib, Necitumumab); drugs that target cells with ALK gene changes (e.g., Crizotinib, Ceritinib, Alectinib, Brigatinib, Lorlatinib); drugs that target cells with ROS1 gene changes (e.g., Crizotinib, Ceritinib, Lorlatinib, Entrectinib); drugs that target cells with BRAF gene changes (e.g., Dabrafenib, Trametinib); drugs that target cells with RET gene changes (e.g., Selpercatinib, Pralsetinib; drugs that target cells with MET gene changes; and drugs that target cells with NTRK gene changes (e.g., Capmatinib, Tepotinib). Targeted therapies for colon cancer include, but are not limited to, drugs that target angiogenesis (e.g., Bevacizumab, Ramucirumab, Ziv-aflibercept); drugs that target cells with EGFR gene changes (e.g., Cetuximab, Panitumumab); drugs that target cells with BRAF gene changes (e.g., Encorafenib); and kinase inhibitors (e.g., Regorafenib). Targeted therapies for breast cancer include, but are not limited to, drugs that target HER2 (e.g., Trastuzumab, Pertuzumab, Margetuximab, and antibody-drug conjugates thereof such as Ado-trastuzumab emtansine and Fam-trastuzumab deruxtecan); kinase inhibitors (e.g., Lapatinib, Neratinib, Tucatinib); CDK4/6 inhibitors (e.g., Palbociclib, ribociclib, abemaciclib); mTOR inhibitors (e.g., Everolimus); PI3K inhibitors (e.g., Alpelisib); drugs that target BRCA gene changes (e.g., Olaparib, Talazoparib); and drugs that target Trop-2 (e.g., Sacituzumab govitecan). Targeted therapies for bladder cancer include, but are not limited to, drugs that target cells with FGFR2 or FGFR3 gene changes (e.g., Balversa). Targeted therapies for renal cell cancer include, but are not limited to, drugs that target angiogenesis (e.g., Sunitinib, Sorafenib, Pazopanib, Cabozantinib, Lenvatinib, Bevacizumab, Axitinib, Tivozanib, Belzutifan); and drugs that target mTOR (e.g., Temsirolimus, Everolimus). Targeted therapies for liver cancer include, but are not limited to, kinase inhibitors (e.g., Sorafenib, Lenvatinib, Regorafenib, Cabozantinib); and drugs that target angiogenesis (e.g., Bevacizumab, Ramucirumab).

In some embodiments, the method further comprises administering a checkpoint inhibitor to the individual. The identification and understanding of immune checkpoint proteins and their role in the immune response represents a breakthrough in cancer therapy. With this discovery, efforts focused on blocking the immune checkpoint pathways in an attempt to activate T-cells directed at cancer cells that were found to be ineffective in stimulating a productive antitumor response. Ipilimumab, an antibody which binds to and functionally blocks CTLA-4, was approved by the US Food and Drug Administration (FDA) for the treatment of metastatic melanoma in 2011. Following on the heels of ipilimumab, antibodies targeting the programmed cell death-1 (PD-1) receptor, and its ligand found on many cancer cells programmed death-ligand 1 (PD-L1), were also approved. These checkpoint inhibitors have led to a revolution in cancer therapy. Tumors that express high amounts of PD-L1 (50% or greater), tumors with a high mutation burden, and tumors with microsatellite instability (MSI) may respond particularly well to checkpoint inhibitors. In some embodiments, the checkpoint inhibitor is administered intravenously. In some embodiments, the immune checkpoint inhibitor is selected from the group consisting of a CTLA-4 pathway antagonist, a PD-1 pathway antagonist, a TIM-3 pathway antagonist, a VISTA pathway antagonist, a BTLA pathway antagonist, a LAG-3 pathway antagonist, and a TIGIT pathway antagonist. In some embodiments, the immune checkpoint inhibitor is an anti-PD-1 antibody, an anti-PD-L1 antibody, an anti-CTLA-4 antibody, an anti-TIM-3 antibody, an anti-BTLA antibody, an anti-B7-H3 antibody, an anti-CD70 antibody, an anti-CD40 antibody, an anti-CD137 antibody, an anti-GITR antibody, an anti-OX40 antibody, an anti-KIR antibody or an anti-LAG-3 antibody. In some embodiments, the checkpoint inhibitor is selected from the group consisting of nivolumab, pembrolizumab, pidilizumab, spartalizumab, MEDI0680, cemiplimab, AMP-224, ipilimumab, BMS-936559, atezolizumab, durvalumab, and avelumab.

In certain embodiments, the methods described herein comprise administering active surveillance. Active surveillance is a treatment plan that involves closely watching a patient's condition but not providing further treatment unless there are changes in test results that show the condition is getting worse. Active surveillance may be used to avoid or delay the need for treatments such as radiation therapy or surgery, which can cause side effects or other problems. During active surveillance, certain examinations and tests are done on a regular schedule, and generally consist of some combination of physical examination, laboratory studies, and imaging. Active surveillance is also called active monitoring. In the case of early stage lung cancer or following curative surgery, for example, active surveillance may comprise chest CTs every two months, every six months, or every year.

The following examples are presented in order to more fully illustrate certain embodiments of the invention. They should in no way, however, be construed as limiting the broad scope of the invention. One skilled in the art can readily devise many variations and modifications of the principles disclosed herein without departing from the scope of the invention.

### EXAMPLES

### Example 1— Pan-Cancer DNA Methylation Markers

Three (3) human genomic loci were identified as DNA methylation markers for the detection of multiple cancer types in cell-free DNA samples (Table 1, SEQ ID NOs: 1-3). These marker loci were found to be hypermethylated in cell-free DNA of cancer patients of various types and stages compared to cell-free DNA of subjects with no cancer.

In addition, three (3) human genomic loci that were previously disclosed by the inventors of the present invention to be hypermethylated in lung cancer DNA compared to normal DNA (Table 1, SEQ ID NOs: 4-6), were found to also have increased methylation levels in cancer types other than lung cancer. More particularly, WO 2019/142193, assigned to the Applicant of the present invention, discloses that sequences comprising or partially overlapping with SEQ ID NOs: 4-6 of the present invention have increased methylation levels in DNA from plasma samples of lung cancer patients compared to DNA from plasma samples of healthy subjects. It is now disclosed that SEQ ID NOs: 4-6 of the present invention also have increased methylation levels in cancer types other than lung cancer. The inventors of the present invention identified that methylation levels of these marker loci above a first threshold are common to a variety of cancer types and stages, and thus serve as a general indication for the presence of cancer in a subject. A further increase in methylation levels, above a second threshold, is specifically indicative for the presence of lung cancer in the subject.

Advantageously, the marker loci are suitable for methylation analysis using methylation-sensitive enzymatic digestion of DNA samples followed by real-time PCR amplification and analysis of the amplification products. More particularly, each marker locus contains at least two restriction sites of methylation-sensitive restriction endonucleases, where the methylation-sensitive restriction endonucleases are HinP1I and AciI. The restriction sites within the marker loci are differentially methylated between cancer and non-cancer DNA. Methylation-sensitive restriction endonucleases cleave their restriction site only if it is unmethylated. Thus, the degree of digestion of each locus by HinP1I and AciI depends on its level of methylation in a tested DNA sample, where increased methylation results in less digestion.

Table 1— Pan-Cancer Methylation Markers

TABLE 1
Pan-cancer methylation markers

| SEQ | | |
|---|---|---|
| ID | | |
| NO. | Nucleic acid sequence* | Position |
| 1 | CCCGCGGCGCCCCCGCGTGTCCCCGCAGCCC | Chromosome 2 |
| | TCCGCGGAAGCAGCGGCGGGAGCGCACCAC | (63134659-63134753) |
| | CTTCACGCGTTCACAGCTGGACGTGCTCGAG | |
| | GCG | |
| 2 | GCGCAAGCGCGCCCCAAGACCATTCTCTTCG | Chromosome 10 |
| | CGC | (22582059-22582092) |
| 3 | GCGGCGGCCCTAAGACGACGCGGGGGTCCT | Chromosome 19 |
| | TGTGCGCACTTAAATGGAGTCGGCTGCGC | (62523441-62523499) |
| 4 | CCGCAGCGCCCGCCACACACCCGCGCCAGA | Chromosome 2 |
| | GGTCCAGCGC | (176712765-176712804) |
| 5 | GCGCGGCGGGCGACAGCCCCCCGGATAACC | Chromosome 17 |
| | CCGCCGAGGGAGGGGCGCTTGTAAAACCGA | (44151843-44151906) |
| | GCGG | |
| 6 | CCGCCGGGTAAATTAGCGGCGAGCCTCGCCA | Chromosome 1 |
| | GACGCTTTCCTCCTTGCCTTCTTTCGCCGAAA | (168907231-168907319) |
| | GGGGGCGCGCTCCTCCCAGGCTGCGC | |

*Analyzed CG sites within each locus, located in recognition sequences of the enzymes used in the assay, are underlined
**Position on hg18 genomic build

Analyzed CG sites within each locus, located in recognition sequences of the enzymes used in the assay, are underlined
*Position on hg18 genomic build

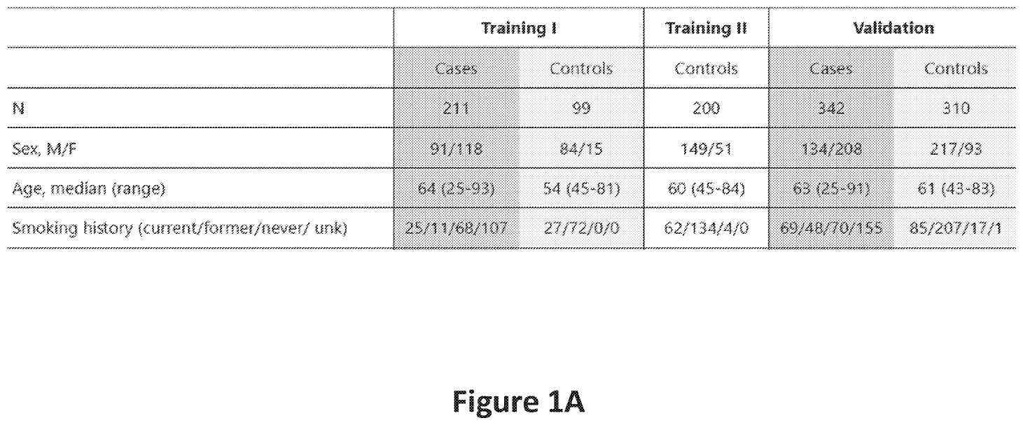

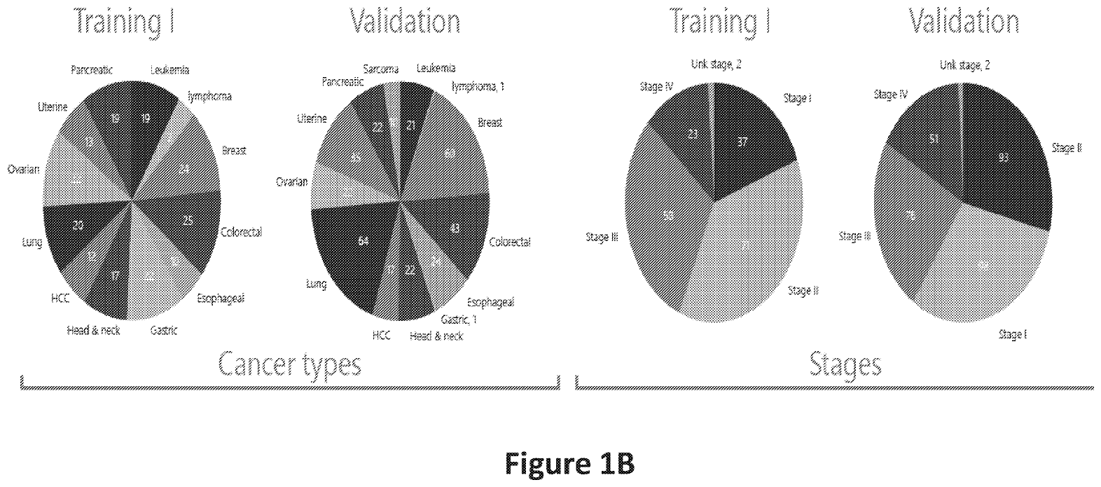

The marker loci were first tested on plasma samples of 211 cancer cases and 99 non-cancer controls, and subsequently on plasma samples of 200 non-cancer controls. The marker loci were then validated on a cohort of 652 samples, of which 342 are cases and 310 controls. Demographic data of the case and control subjects are provided in FIG. 1A. FIG. 1B shows cancer distribution of the cancer patients by type and stage.

The cancer and non-cancer plasma samples were processed in a similar manner, as follows:

DNA was extracted from the plasma samples using the QIAamp® Circulating Nucleic Acid Kit (QIAGEN, Hilden, Germany). Each extracted DNA sample was subjected to digestion with HinP1I and AciI. The digestion reaction (total volume 100 microliter) included 80 microliters of the extracted DNA (not quantified), HinP1I and AciI in a digestion buffer. Enzymatic digestion was performed for 2 hours at 37° C. followed by a heat inactivation of the enzymes for 20 minutes at 65° C.

Next, real-time PCR was carried out on the digested DNA samples to amplify in each sample the six (6) marker loci and the following control locus (SEQ ID NO: 7), which does not contain a recognition sequence of HinP1I and AciI (does not contain a nucleotide sequence recognized by any of these enzymes, whether methylated or not) and remains intact upon digestion with HinP1I and AciI regardless of its methylation status:

SEQ ID NO: 7:
AGACTAACTTTTCTCTTGTACAGAATCATCAGGCTAAATTTTTGGC
ATTATTTCAGTCCT

More particularly, each digested DNA sample was divided into three (3) aliquots containing 12 microliters of the digested DNA. Each aliquot was supplemented with primer pairs for amplification of two marker loci out of the six and the control locus (the control locus was amplified in every aliquot). Amplicons of between 93 to 140 bases were amplified. Each amplification reaction (total volume 30 microliter) further contained dNTPs, a DNA polymerase and a reaction buffer. To enable detection of amplification products during amplification, fluorescently-labeled polynucleotide probes (one for each locus) were added to the reaction. Real-time PCR reactions were carried out in an ABI 7500 FastDx instrument with the following PCR program: 95° C., 10 min->45X(95° C., 15 sec)->60° C., 1 min. Following amplification, data on the level of fluorescent signals from the probes as a function of cycle number were analyzed to calculate the quantification cycle (Cq) for each locus, and a ΔCq between the Cq of the control locus and the Cq of a marker locus was calculated for each marker locus and used in the following formula:

2^{(Cq of control locus—Cq of marker locus)}

It is to be understood that the calculation was performed for a marker locus and a control locus that were co-amplified in the same aliquot.

The numerical value obtained for a given marker locus with respect to the control locus represents a ratio between the signal intensities of the amplification products of this marker locus and the control locus, and reflects the methylation ratio between this marker locus and the control locus in the DNA sample. Next, a marker score was calculated for each marker locus, the score being the signal ratio normalized in respect to reference ratios such that the highest signal ratio is scored “100” and the lowest signal ratio is scored “0”. Altogether, six marker scores were calculated for each DNA sample. The six individual marker scores obtained for each DNA sample were combined into a single sample score, termed “EpiScore”, which is a number between 0 and 100, reflecting the overall relative methylation level of the DNA sample at the panel of six marker loci. A threshold score was set, above which the DNA sample was classified as positive for cancer. An EpiScore below the threshold classified the DNA sample as negative for cancer.

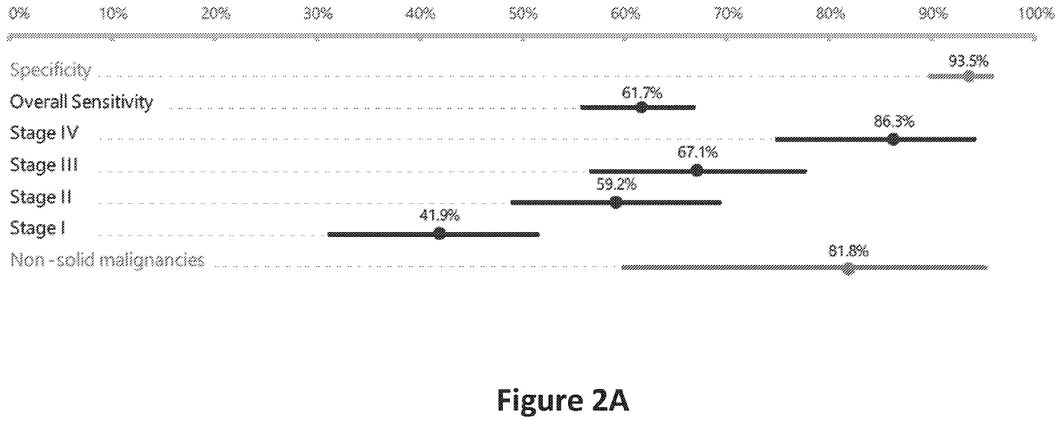

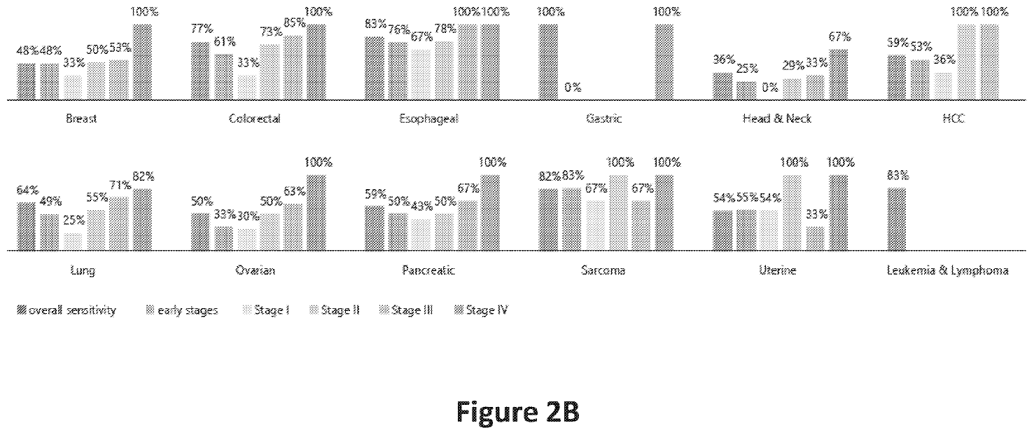

Sensitivity and specificity with respect to the detection of cancer were calculated for the panel of six marker loci. The results are summarized in FIGS. 2A-2B.

The marker loci showed an overall sensitivity (irrespective of cancer type and stage) of 62%, and an overall specificity of 94%. Highest sensitivity was demonstrated in GI cancers (77% colorectal, 83% esophageal, 100% gastric) and non-solid malignancies (83%). It is noted that to date, most GI cancers, as well as non-solid malignancies, have no available screening option, let alone non-invasive screening.

Thus, with only six markers the presence of multiple types of cancer could be detected in a single assay.

Remarkably, the panel of markers was found to be particularly sensitive for early-stage cancers (stages I, II & IIIA), with above 40% sensitivity for stage I cancer, and 52% sensitivity for stages I, II & IIIA. These results demonstrate the ability of the marker loci to detect the presence of various types of cancers at a stage at which no symptoms or only mild symptoms are present. Some of the cancer types detected by the marker loci at an early stage are typically diagnosed only when significant symptoms are present and treatment options are limited.

### Example 2— Tissue-Specificity DNA Methylation Markers

A set of genomic loci were identified as DNA methylation markers for distinguishing between lung cancer vs. four other cancer types: colorectal, liver, breast and hematological cancers (Table 2, SEQ ID NOs: 5, 8-15). As noted above, the inventors of the present invention identified that a certain increase in the methylation level of SEQ ID NO: 5 is common to a variety of cancer types and stages and therefore provides a general indication that cancer is present in the subject. A further increase in the methylation level is characteristic of lung cancer. The inventors of the present invention identified a threshold value that distinguishes between lung cancer and colorectal cancer.

Advantageously, these tissue-specificity marker loci are suitable for methylation analysis using methylation-sensitive enzymatic digestion of DNA samples followed by real-time PCR amplification and analysis of the amplification products. More particularly, each tissue-specificity marker locus contains at least two restriction sites of methylation-sensitive restriction endonucleases, where the methylation-sensitive restriction endonucleases are HinP1I and AciI. The restriction sites within the marker loci are differentially methylated between different types of cancer. Methylation-sensitive restriction endonucleases cleave their restriction site only if it is unmethylated. Thus, the degree of digestion of each locus by HinP1I depends on its level of methylation in a tested DNA sample, where increased methylation results in less digestion.

Table 2— Tissue-specificity methylation markers

TABLE 2
Tissue-specificity methylation markers

| SEQ | | | |
|---|---|---|---|
| ID | | | |
| NO. | Nucleic acid sequence* | Position | Description |
| 5 | GCGCGGCGGGCGACAGCCCCCCG | Chromosome 17 | Hypermethylated |
| | GATAACCCCGCCGAGGGAGGGGC | (44151843- | in lung cancer vs. |
| | GCTTGTAAAACCGAGCGG | 44151906) | colorectal |
| 8 | GCGCCCGCGCCCCCATTGGCCGTG | Chromosome 7 | Hypermethylated |
| | CGCGTCACGTGCCCGTCCAGCAG | (27171718- | in lung cancer vs. |
| | AACAATAACGCGTAAATCACTCC | 27171789) | colorectal |
| | GC | | |
| 9 | GCGGATAAGGGTGTGCGCGAGAG | Chromosome 11 | Hypermethylated |
| | CCAATCAAAAGCGGATGTCGAAA | (13646665- | in liver cancer vs. |
| | AGAGGCGCCGC | 13646721) | lung cancer |
| 10 | GCGCGGTGAACGTGGGGGTTGAA | Chromosome 17 | Hypermethylated |
| | ACGCTCCACGCGGAAGGTAGAGG | (45405239- | in lung cancer vs. |
| | GCAGGGGCCAAGGGGGCGATCCT | 45405318) | liver cancer |
| | GGTGGCTGCGC | | |
| 11 | CCGCGGAGCTGCAAGGGGGGGCG | Chromosome 4 | Hypermethylated |
| | GTTTCTCACCCGCCCCGAGAGCGC | (76774710- | in breast cancer |
| | | 76774756) | vs. lung cancer |
| 12 | GCGCTTTCTTGGGTCCCCCATTCC | Chromosome 1 | Hypermethylated |
| | CCCAGGTTAGAGCGCGGCTCCAG | (29458817- | in lung cancer vs. |
| | GAACCTATGTCCGCGCGG | 29458881) | breast cancer |
| 13 | GCGCTTGCGCCCGGTTCAGGACC | Chromosome 1 | Hypermethylated |
| | GGGTCCAAATGAGGTTGGCGTGC | (226713528- | in hematological |
| | GCCGTGCAGGTGAGGAGTGCGG | 226713595) | cancer vs. lung |
| | | | cancer |
| 14 | CCGCTTCCCCGGTCTTTGCCAAGT | Chromosome 9 | Hypermethylated |
| | GGGTTTACGAGGTCTGAGGCGTG | (108662393- | in hematological |
| | TTGGGGCGCGC | 108662450) | cancer vs. lung |
| | | | cancer |
| 15 | GCGGGCGGGCTGCGCACAGCGGG | Chromosome 16 | Hypermethylated |
| | GACAAGGCTGCCCCCTTCCTCCTC | (11234673- | in lung cancer vs. |
| | CGCTGCCTCCGCGGCCGC | 11234737) | hematological |
| | | | cancer |

*Analyzed CG sites within each locus, located in recognition sequences of the enzymes used in the assay, are underlined
**Position on hg18 genomic build

Analyzed CG sites within each locus, located in recognition sequences of the enzymes used in the assay, are underlined
* Position on hg18 genomic build

Lung Vs. Colorectal

Genomic loci SEQ ID NO: 5 and SEQ ID NO: 8 were identified as tissue-specificity DNA methylation markers that distinguish between lung cancer and colorectal cancer. These tissue-specificity marker loci were tested on 28 plasma samples of lung cancer patients and 29 plasma samples of colorectal cancer patients that were classified as ‘cancer positive’ using the pan-cancer marker loci described in Example 1. DNA extracted from the plasma samples was subjected to digestion by HinP1I and AciI followed by co-amplification of the marker loci and a control locus as described in Example 1, to obtain a ΔCq between the Cq of the control locus and the Cq of a marker locus for each tissue-specificity marker locus.

Next, a combined ΔCq value was calculated for each sample using the ΔCq of SEQ ID NO: 5 with respect to the control locus (ΔCq_{SEQ ID NO: 5}) and the ΔCq of SEQ ID NO: 8 with respect to the control locus (ΔCq_{SEQ ID NO: 8}). Since both loci are more methylated in lung cancer than in colorectal cancer, a sum of their ΔCq values was calculated, as follows:

ΔCq_{SEQ ID NO: 5}+ΔCq_{SEQ ID NO: 8}

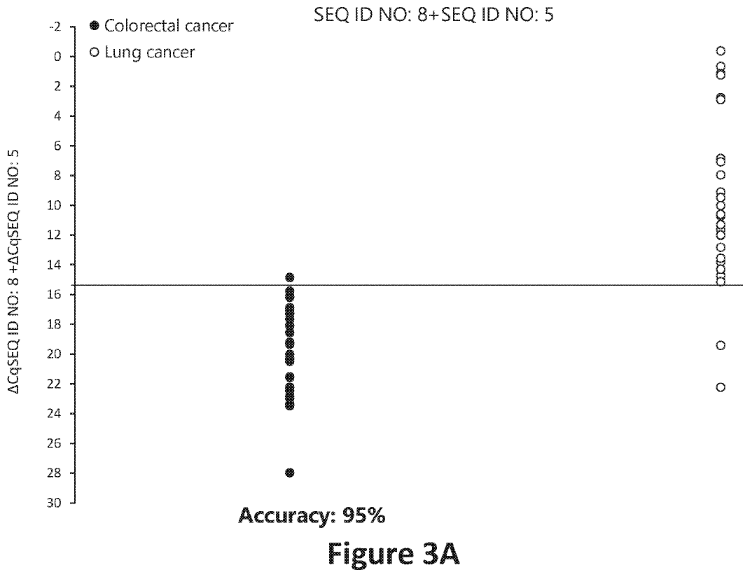

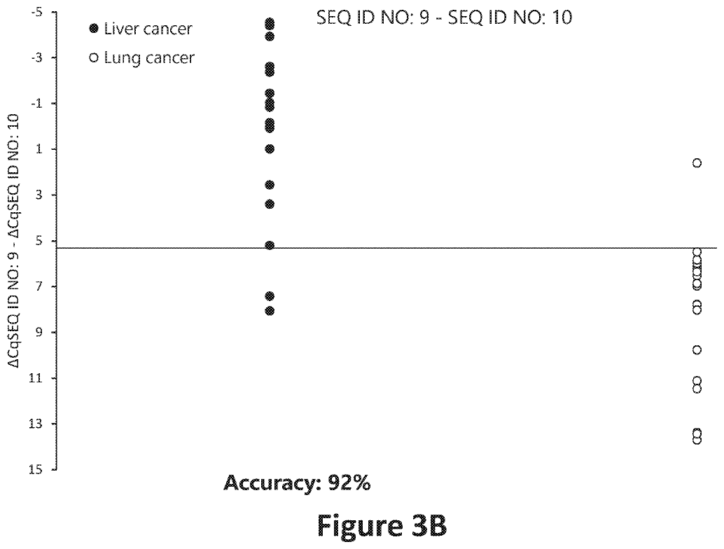

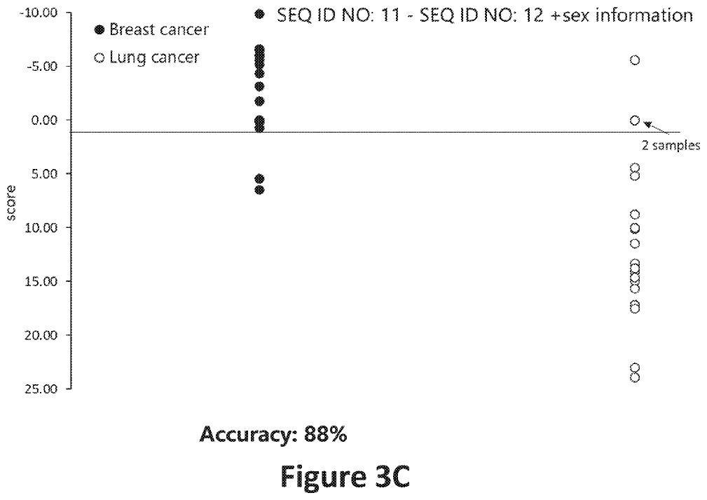

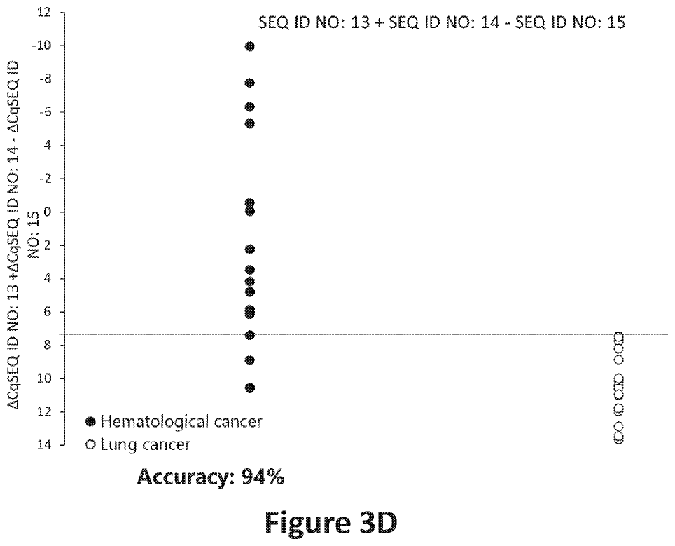

FIG. 3A shows a plot of the combined ΔCq values calculated for the samples, grouped according to the cancer type. A threshold value was set (horizontal line in FIG. 3A). A combined ΔCq value above the threshold classified the sample as colorectal cancer. A combined ΔCq value below the threshold classified the sample as lung cancer.

The tissue specificity marker loci SEQ ID NO: 5 and SEQ ID NO: 8 correctly classified the samples with an accuracy of 95%.

Lung Vs. Liver

Genomic loci SEQ ID NO: 9 and SEQ ID NO: 10 were identified as tissue-specificity DNA methylation markers that distinguish between lung cancer and liver cancer.

These tissue-specificity marker loci were tested on 20 plasma samples of lung cancer patients and 18 plasma samples of liver cancer patients that were classified as ‘cancer positive’ using the pan-cancer marker loci described in Example 1. DNA extracted from the plasma samples was subjected to digestion by HinP1I and AciI followed by co-amplification of the marker loci and the control locus as described in Example 1, to obtain a ΔCq between the Cq of the control locus and the Cq of a marker locus for each tissue-specificity marker locus.

Next, a combined ΔCq value was calculated for each sample using the ΔCq of SEQ ID NO: 9 with respect to the control locus (ΔCq_{SEQ ID NO: 9}) and the ΔCq of SEQ ID NO: 10 with respect to the control locus (ΔCq_{SEQ ID NO: 10}). SEQ ID NO: 9 is more methylated in liver cancer than in lung cancer, whereas SEQ ID NO: 10 is more methylated in lung cancer than in liver cancer. Thus, a difference between their ΔCq values was calculated, as follows:

ΔCq_{SEQ ID NO: 9}−ΔCq_{SEQ ID NO: 10}

FIG. 3B shows a plot of the combined ΔCq values calculated for the samples, grouped according to the cancer type. A threshold value was set (horizontal line in FIG. 3B). A combined ΔCq value above the threshold classified the sample as lung cancer. A combined ΔCq value below the threshold classified the sample as liver cancer.

The tissue specificity marker loci SEQ ID NO: 9 and SEQ ID NO: 10 correctly classified the samples with an accuracy of 92%.

Lung Vs. Breast

Genomic loci SEQ ID NO: 11 and SEQ ID NO: 12 were identified as tissue-specificity DNA methylation markers that distinguish between lung cancer and breast cancer. These tissue-specificity marker loci were tested on 20 plasma samples of lung cancer patients and 20 plasma samples of breast cancer patients that were classified as ‘cancer positive’ using the pan-cancer marker loci described in Example 1. DNA extracted from the plasma samples was subjected to digestion by HinP1I and AciI followed by co-amplification of the marker loci and the control locus as described in Example 1, to obtain a ΔCq between the Cq of the control locus and the Cq of a marker locus for each tissue-specificity marker locus.

Next, a combined ΔCq value was calculated for each sample using the ΔCq of SEQ ID NO: 11 with respect to the control locus (ΔCq_{SEQ ID NO: 11}) and the ΔCq of SEQ ID NO: 12 with respect to the control locus (ΔCq_{SEQ ID NO: 12}). SEQ ID NO: 11 is more methylated in breast cancer than in lung cancer, whereas SEQ ID NO: 12 is more methylated in lung cancer than in breast cancer. Thus, a difference between their ΔCq values was calculated, as follows:

ΔCq_{SEQ ID NO: 11}−ΔCq_{SEQ ID NO: 12}

A score was calculated for each sample based on the combined ΔCq value calculated for the sample and gender information.

FIG. 3C shows a plot of the scores calculated for the samples, grouped according to the cancer type. A threshold score was set (horizontal line in FIG. 3C). A score above the threshold classified the sample as lung cancer. A score below the threshold classified the sample as breast cancer.

The tissue specificity marker loci SEQ ID NO: 11 and SEQ ID NO: 12 correctly classified the samples with an accuracy of 88%.

Lung Vs. Hematological Cancers

Genomic loci SEQ ID NO: 13, SEQ ID NO: 14 and SEQ ID NO: 15 were identified as tissue-specificity DNA methylation markers that distinguish between lung cancer and hematological cancers. These tissue-specificity marker loci were tested on 17 plasma samples of lung cancer patients and 16 plasma samples of hematological cancer patients (12 lymphoma and 4 acute lymphoblastic leukemia) that were classified as ‘cancer positive’ using the pan-cancer marker loci described in Example 1. DNA extracted from the plasma samples was subjected to digestion by HinP1I and AciI followed by co-amplification of the marker loci and the control locus as described in Example 1, to obtain a ΔCq between the Cq of the control locus and the Cq of a marker locus for each tissue-specificity marker locus.

Next, a combined ΔCq value was calculated for each sample

SEQ ID NO: 13 and SEQ ID NO: 14 are more methylated in hematological cancer than in lung cancer, whereas SEQ ID NO: 15 is more methylated in lung cancer than in hematological cancer. Thus, the ΔCq values of SEQ ID NO: 13 and SEQ ID NO: 14 were summed, and the ΔCq value of SEQ ID NO: 15 was subtracted from the sum, as follows:

ΔCq_{SEQ ID NO: 13}+ΔCq_{SEQ ID NO: 14}−ΔCq_{SEQ ID NO: 15}

FIG. 3D shows a plot of the combined ΔCq values calculated for the samples, grouped according to the cancer type. A threshold value was set (horizontal line in FIG. 3D). A combined ΔCq value above the threshold classified the sample as lung cancer. A combined ΔCq value below the threshold classified the sample as a hematological cancer.

The tissue specificity marker loci SEQ ID NO: 13, SEQ ID NO: 14 and SEQ ID NO: 15 correctly classified the samples with an accuracy of 94%.

### Example 3— Assessing the Presence of Cancer and Indicating a Tissue Source Using Next-Generation Sequencing (NGS)

Cell-free DNA samples are digested with methylation-sensitive restriction endonucleases. Next, a sequencing library is prepared by enriching in each sample DNA fragments corresponding to marker and control loci, and introducing sequencing adapters into these DNA fragments. In the current example, the sequencing adapters are introduced using a 2-step PCR, wherein the first PCR is carried out using primers that contain locus-specific sequences and overhang sequences that introduce a first portion of the sequencing adapters, and the second PCR is carried out using primers that introduce a second portion of the sequencing adapters and sample indices.

For the locus-specific PCR, primers are designed which contain overhang adapter sequences in addition to locus-specific sequences. The overhang adapter sequences are 5′ of the locus specific sequences, and they introduce 5′ overhang adapter sequences into the PCR products. In between the overhang adapter sequences and the locus-specific sequences, either 0, 1, 2 or 3 N nucleotides are added. This addition is done in order to ensure even distribution of all four bases in each sequencing cycle. These N nucleotides are added to both forward and revers primers. Overall, there are four forward and four revers primers for each locus.

DNA is extracted from plasma samples of cancer cases and controls using the QIAamp® Circulating Nucleic Acid Kit (QIAGEN, Hilden, Germany). Each extracted DNA sample is subjected to digestion with HinP1I and AciI. The digestion reaction (total volume 100 microliter) includes 80 microliters of the extracted DNA (not quantified), HinP1I and AciI in a digestion buffer. Enzymatic digestion is performed for 2 hours at 37° C. followed by a heat inactivation of the enzymes for 20 minutes at 65° C.

NGS library is prepared by a 2-step PCR, as follows:

The first PCR is a locus-specific PCR. The pan-cancer marker loci, tissue-specificity marker loci and the control locus are amplified in each digested DNA sample using locus-specific primers that contain the 5′ overhang adapter sequences described above and 0-3 N nucleotides between the locus-specific sequences and the 5′ overhang adapter sequences. 10 μl of digested DNA are amplified in a 25-cycles PCR reaction using a high-fidelity DNA polymerase. The digested DNA includes high concentrations of magnesium. Magnesium is also present in the PCR reaction buffer of the high-fidelity DNA polymerase. In order to avoid excess magnesium that might inhibit the PCR reaction, the magnesium concentration in the amplification reaction is adjusted according to the DNA polymerase that is used.

After the locus-specific PCR, the PCR products are purified using AMPure XP beads (Beckman Coulter Genomics), and a second PCR is carried out to introduce Illumina adapters and sample indices. The second PCR is performed using primers from Nextera NT index kit (Illumina). 5 μl from the purified products of the first PCR are taken for an 8-cycles PCR reaction that includes also 5 μl from each Nextera primer (N7 and S5) and 25 μl KAPA HiFi HotStart Ready Mix (KAPA Biosystems). After the second PCR, the PCR products are purified using AMPure XP beads (Beckman Coulter Genomics).

The purified PCR products are quantified using Qubit 3.0 fluorometer and dsDNA high sensitivity assay kit (Thermo Fisher Scientific). The purified PCR products are diluted to 1 nM. Next, 5 μl from each diluted PCR product are pooled together to create a pooled library sample. The DNA library is sequenced on iSeq 100 system (Illumina) using iSeq 100 Reagent kit. 45 pM of a pooled library sample+20% PhiX library (Illumina) is inserted to the iSeq 100 flow cell.

The sequence reads are analyzed using a computer software to determine a read count (copy number) for each of the pan-cancer marker loci, tissue specificity marker loci and the control locus.

Relative copy number is calculated for each marker locus in each sample, as follows:

Relative copy number=Read count of marker locus/Read count of control locus

The read count of each marker locus reflects the number of methylated copies of this locus that were present in the tested DNA sample (the methylated copies remain intact when the sample is digested with methylation-sensitive restriction endonucleases). The relative copy number for each marker locus represents a methylation value of the marker locus. The relative copy numbers of the pan-cancer marker loci and the tissue specificity marker loci are used for assessing whether a tested DNA sample is from a subject with cancer or from a healthy subject, and for indicating the tissue source of the cancer, by comparison to relative copy numbers calculated in one or more reference DNA samples.

The foregoing description of the specific embodiments will so fully reveal the general nature of the invention that others can, by applying current knowledge, readily modify and/or adapt for various applications such specific embodiments without undue experimentation and without departing from the generic concept, and therefore, such adaptations and modifications should and are intended to be comprehended within the meaning and range of equivalents of the disclosed embodiments. It is to be understood that the phraseology or terminology employed herein is for the purpose of description and not of limitation. The means, materials, and steps for carrying out various disclosed chemical structures and functions may take a variety of alternative forms without departing from the invention.

1. A method for assessing the presence of cancer in a human subject, the method comprising:
(i) determining in a cell-free DNA (cfDNA) sample of the subject a methylation value for at least one marker locus that is hypermethylated in cancer DNA compared to non-cancer DNA selected from the group consisting of SEQ ID NO: 1, SEQ ID NO: 2 and SEQ ID NO: 3; and
(ii) comparing the methylation value of said at least one marker locus to at least one reference methylation value selected from a cancer methylation value and a non-cancer methylation value, to determine the likelihood that the subject has cancer.

2. The method of claim 1, wherein the cfDNA sample is cfDNA extracted from a plasma sample.

3. The method of any one of the preceding claims, wherein the at least one marker locus comprises a plurality of marker loci selected from the group consisting of SEQ ID NO: 1, SEQ ID NO: 2 and SEQ ID NO: 3.

4. The method of any one of the preceding claims, wherein step (a) further comprises determining in the cfDNA sample of the subject a methylation value for at least one marker locus that is hypermethylated in cancer DNA compared to non-cancer DNA selected from the group consisting of SEQ ID NO: 4, SEQ ID NO: 5 and SEQ ID NO: 6.

5. The method of any one of the preceding claims, wherein step (a) comprises determining in the cfDNA sample of the subject a methylation value for each of the marker loci SEQ ID NO: 1, SEQ ID NO: 2, SEQ ID NO: 3, SEQ ID NO: 4, SEQ ID NO: 5 and SEQ ID NO: 6.

6. The method of any one of the preceding claims, wherein the type of cancer the presence of which can be assessed comprises at least one of lung cancer, breast cancer, colorectal cancer, hepatocellular carcinoma (HCC), leukemia, lymphoma, esophageal cancer, gastric cancer, head and neck cancer, ovarian cancer, uterine cancer, pancreatic cancer and sarcoma.

7. The method of any one of the preceding claims, wherein the methylation value is determined by a methylation mapping comprising one or more analysis methods selected from the group consisting of DNA sequencing, real-time PCR, and array hybridization.

8. The method of any one of the preceding claims, wherein determining the methylation value comprises, for a plurality of potential methylation sites in a plurality of marker loci, quantifying methylated read counts at the potential methylation sites and calculating a methylation value based on the number of methylated read counts.

9. The method of any one of the preceding claims, wherein determining the methylation value comprises, for a plurality of marker loci, determining a ΔCq for each marker locus and calculating a single composite methylation value based on the ΔCq determined for each marker locus.

10. The method of any one of the preceding claims, comprising:
subjecting the cfDNA sample to digestion with at least one methylation-sensitive restriction endonuclease recognizing a sequence within the at least one marker locus that is hypermethylated in cancer DNA compared to non-cancer DNA, thereby obtaining restriction endonuclease-treated DNA;
co-amplifying from the restriction endonuclease-treated DNA the at least one marker locus and a control locus, thereby generating an amplification product for each locus;
determining a signal intensity for each generated amplification product; and
comparing a ratio between the signal intensities of the amplification products of each of said at least one marker locus and the control locus to at least one reference ratio selected from cancer reference ratio and non-cancer reference ratio, to determine likelihood that the subject has cancer.

11. The method of claim 10, wherein the step of subjecting the cfDNA sample to digestion with at least one methylation-sensitive restriction endonuclease is performed using a single methylation-sensitive restriction endonuclease.

12. The method of claim 11, wherein the methylation-sensitive restriction endonuclease is HinP1I.

13. The method of claim 10, wherein the step of subjecting the cfDNA sample to digestion with at least one methylation-sensitive restriction endonuclease is performed using a plurality of methylation-sensitive restriction endonucleases.

14. The method of claim 13, wherein the plurality of methylation-sensitive restriction endonucleases comprises HinP1I.

15. The method of any one of claims 10-14, wherein the control locus is a locus that does not contain a nucleotide sequence recognized by the methylation-sensitive restriction endonuclease.

16. The method of claim 15, wherein the at least one methylation-sensitive restriction endonuclease comprises HinP1I and the control locus is SEQ ID NO: 7.

17. The method of any one of claims 10-16, wherein the cfDNA subjected to digestion with the at least one methylation-sensitive restriction endonuclease comprises less than 1% ssDNA and contains at least 3.5 ng cfDNA.

18. The method of any one of claims 10-17, wherein the amplification product for each locus is between about 90 and about 150 bases in length.

19. The method of any one of claims 10-18, wherein the step of co-amplifying from the restriction endonuclease-treated DNA the at least one marker locus and a control locus is performed using real-time PCR.

20. The method of claim 19, wherein the step of co-amplifying from the restriction endonuclease-treated DNA the at least one marker locus and a control locus comprises adding fluorescent probes for assisting in detecting the amplification products of the at least one marker locus and the control locus.

21. The method of claim 20, wherein the ratio between the signal intensities of the amplification products of each of said at least one marker locus and the control locus is calculated by determining the quantification cycle (Cq) for each locus and calculating 2^{(Cq control locus—Cq marker locus)}.

22. The method of any one of the preceding claims, further comprising providing an indication of the tissue source of the cancer for a subject with a positive assessment of having cancer.

23. The method of claim 22, wherein providing an indication of the tissue source of the cancer comprises analyzing the methylation of at least one tissue-specificity marker locus selected from:
(i) one or more tissue specificity marker locus distinguishing between lung cancer and colorectal cancer selected from SEQ ID NO: 5 and SEQ ID NO: 8;
(ii) one or more tissue specificity marker locus distinguishing between lung cancer and liver cancer selected from SEQ ID NO: 9 and SEQ ID NO: 10;
(iii) one or more tissue specificity marker locus distinguishing between lung cancer and breast cancer selected from SEQ ID NO: 11 and SEQ ID NO: 12; and
(iv) one or more tissue specificity marker locus distinguishing between lung cancer and hematological cancer selected from SEQ ID NO: 13, SEQ ID NO: 14 and SEQ ID NO: 15.

24. The method of claim 23, wherein analyzing the methylation of at least one tissue-specificity marker locus comprises:
(i) determining in the cfDNA sample a methylation value for the one or more tissue specificity marker locus distinguishing between lung cancer and colorectal cancer selected from SEQ ID NO: 5 and SEQ ID NO: 8;
(ii) comparing the methylation value of each of said one or more tissue specificity marker locus to a corresponding lung cancer reference methylation value and a corresponding colorectal cancer reference methylation value; and
(iii) determining, based on the comparison, whether the methylation value of the one or more tissue specificity marker locus determined for the cfDNA sample represents a lung cancer value or a colorectal cancer value.

25. The method of claim 23, wherein analyzing the methylation of at least one tissue-specificity marker locus comprises:
(i) determining in the cfDNA sample a methylation value for the one or more tissue specificity marker locus distinguishing between lung cancer and liver cancer selected from SEQ ID NO: 9 and SEQ ID NO: 10;
(ii) comparing the methylation value of each of said one or more tissue specificity marker locus to a corresponding lung cancer reference methylation value and a corresponding liver cancer reference methylation value; and
(iii) determining, based on the comparison, whether the methylation value of the one or more tissue specificity marker locus determined for the cfDNA sample represents a lung cancer value or a liver cancer value.

26. The method of claim 23, wherein analyzing the methylation of at least one tissue-specificity marker locus comprises:
(i) determining in the cfDNA sample a methylation value for the one or more tissue specificity marker locus distinguishing between lung cancer and breast cancer selected from SEQ ID NO: 11 and SEQ ID NO: 12;
(ii) comparing the methylation value of each of said one or more tissue specificity marker locus to a corresponding lung cancer reference methylation value and a corresponding breast cancer reference methylation value; and
(iii) determining, based on the comparison, whether the methylation value of the one or more tissue specificity marker locus determined for the cfDNA sample represents a lung cancer value or a breast cancer value.

27. The method of claim 26, further comprising calculating a breast cancer score based on the methylation value of the one or more tissue specificity marker locus and gender information of the subject, and determining whether the cfDNA sample represents a lung cancer sample or a breast cancer sample based on the breast cancer score.

28. The method of claim 23, wherein analyzing the methylation of at least one tissue-specificity marker locus comprises:
(i) determining in the cfDNA sample a methylation value for the one or more tissue specificity marker locus distinguishing between lung cancer and hematological cancer selected from the group consisting of SEQ ID NO: 13, SEQ ID NO: 14 and SEQ ID NO: 15;
(ii) comparing the methylation value of each of said one or more tissue specificity marker locus to a corresponding lung cancer reference methylation value and a corresponding hematological cancer reference methylation value; and
(iii) determining, based on the comparison, whether the methylation value of the one or more tissue specificity marker locus determined for the cfDNA sample represents a lung cancer value or a hematological cancer value.

29. The method of any one of claims 24-28, wherein step (i) comprises determining a methylation value for each tissue specificity marker locus and subsequently calculating a combined methylation value based on the individual methylation values, and wherein following steps (ii) and (iii) are carried out using the combined methylation value.

30. The method of any one of claims 24-29, wherein step (ii) comprises comparing to a threshold value differentiating between the tissue sources, and step (iii) comprises determining which tissue source the methylation value represents based on the methylation value being above or below the threshold.

31. The method of any one of claims 24-29, wherein step (iii) comprises determining a likelihood score reflecting the likelihood that the cfDNA sample is of a particular tissue source based on the comparison to the corresponding reference values.

32. The method of claim 23, wherein analyzing the methylation of at least one tissue-specificity marker locus comprises:
(i) determining in the cfDNA sample a methylation value for each of the marker loci SEQ ID NO: 5, SEQ ID NO: 8, SEQ ID NO: 9, SEQ ID NO: 10, SEQ ID NO: 11, SEQ ID NO: 12, SEQ ID NO: 13, SEQ ID NO: 14 and SEQ ID NO: 15;
(ii) determining a plurality of likelihood scores comprising:
a likelihood score reflecting the likelihood that the tissue source is lung cancer and not colorectal cancer based on comparison of the methylation values of the marker loci SEQ ID NO: 5 and SEQ ID NO: 8 to corresponding lung cancer and colorectal cancer reference methylation values;
a likelihood score reflecting the likelihood that the tissue source is lung cancer and not liver cancer based on comparison of the methylation values of the marker loci SEQ ID NO: 9 and SEQ ID NO: 10 to corresponding lung cancer and liver cancer reference methylation values;
a likelihood score reflecting the likelihood that the tissue source is lung cancer and not breast cancer based on comparison of the methylation values of the marker loci SEQ ID NO: 11 and SEQ ID NO: 12 to corresponding lung cancer and breast cancer reference methylation values;
a likelihood score reflecting the likelihood that the tissue source is lung cancer and not a hematological cancer based on comparison of the methylation values of the marker loci SEQ ID NO: 13, SEQ ID NO: 14 and SEQ ID NO: 15 to corresponding lung cancer and hematological cancer reference methylation values; and
(iii) determining a lung cancer score based on the plurality of likelihood scores, reflecting the likelihood that the tissue source is lung cancer.

33. The method of any one of the preceding claims, further comprising preparing a report in paper or electronic form based on the assessment of the presence of cancer and optionally the indicated tissue source of the cancer, and optionally communicating the report to the subject and/or a healthcare provider of the subject.

34. A method for profiling methylation of a cell-free DNA (cfDNA) sample of a human subject, the method comprising:
(i) determining in the cfDNA sample a methylation value for at least one marker locus hypermethylated in cancer DNA compared to non-cancer DNA selected from the group consisting of SEQ ID NO: 1, SEQ ID NO: 2 and SEQ ID NO: 3;
(ii) determining for each of said at least one marker locus whether its methylation value represents a cancer methylation value or a non-cancer methylation value, based on a comparison to at least one reference methylation value selected from a cancer reference value and a non-cancer reference value,
thereby profiling methylation of the cfDNA sample.

35. The method of claim 34, wherein step (a) further comprises determining in the cfDNA sample of the subject a methylation value for at least one marker locus hypermethylated in cancer DNA compared to non-cancer DNA selected from the group consisting of SEQ ID NO: 4, SEQ ID NO: 5 and SEQ ID NO: 6.

36. The method of any one of claims 34-35, further comprising profiling methylation of one or more tissue-specificity marker loci, said profiling comprising at least one of:
(i) determining in the cfDNA sample a methylation value for at least one marker locus selected from SEQ ID NO: 5 and SEQ ID NO: 8, and determining for each of said at least one marker locus whether its methylation value represents a lung cancer methylation value or a colorectal cancer methylation value, based on a comparison to corresponding lung cancer and colorectal cancer reference values;
(ii) determining in the cfDNA sample a methylation value for at least one marker locus selected from SEQ ID NO: 9 and SEQ ID NO: 10, and determining for each of said at least one marker locus whether its methylation value represents a lung cancer methylation value or a liver cancer methylation value, based on a comparison to corresponding lung cancer and liver cancer reference values;
(iii) determining in the cfDNA sample a methylation value for at least one marker locus selected from SEQ ID NO: 11 and SEQ ID NO: 12, and determining for each of said at least one marker locus whether its methylation value represents a lung cancer methylation value or a breast cancer methylation value, based on a comparison to corresponding lung cancer and breast cancer reference values; and
(iv) determining in the cfDNA sample a methylation value for at least one marker locus selected from SEQ ID NO: 13, SEQ ID NO: 14 and SEQ ID NO: 15, and determining for each of said at least one marker locus whether its methylation value represents a lung cancer methylation value or a hematological cancer methylation value, based on a comparison to corresponding lung cancer and hematological cancer reference values,
thereby profiling methylation of the cfDNA sample.

37. A method for profiling methylation of a cell-free DNA (cfDNA) sample of a human subject, the method comprising determining in the cell-free DNA (cfDNA) sample a methylation value for at least one marker locus preferably, two loci, and most preferably three loci selected from the group consisting of SEQ ID NO: 1, SEQ ID NO: 2 and SEQ ID NO: 3.

38. The method of claim 37, wherein the methylation value is determined by a methylation mapping comprising one or more analysis methods selected from the group consisting of DNA sequencing, real-time PCR, and array hybridization.

39. The method of claim 37, wherein determining the methylation value comprises, for a plurality of potential methylation sites in a plurality of marker loci, quantifying methylated read counts at the potential methylation sites and calculating a methylation value based on the number of methylated read counts.

40. The method of claim 37, wherein determining the methylation ratio comprises:
(i) subjecting the cfDNA to digestion with at least one methylation-sensitive restriction endonuclease recognizing a sequence within the at least one marker locus, thereby obtaining restriction endonuclease-treated DNA;
(ii) co-amplifying from the restriction endonuclease-treated DNA the at least one marker locus and a control locus, thereby generating an amplification product for each locus;
(iii) determining a signal intensity for each generated amplification product; and
(iv) calculating a ratio between the signal intensities of the amplification products of each of said at least one restriction locus and the control locus, thereby measuring a methylation ratio for the at least one marker locus,
thereby profiling methylation of the cfDNA sample.

41. The method of claim 40, wherein determining the methylation value comprises, for a plurality of marker loci, determining a ΔCq for each marker locus and calculating a single composite methylation value based on the ΔCq determined for each marker locus.

42. The method of claim 40, wherein the cfDNA subjected to digestion with the at least one methylation-sensitive restriction endonuclease comprises less than 1% ssDNA and contains at least 3.5 ng cfDNA, and/or wherein the amplification product for each locus is between about 90 and about 150 bases in length.

43. The method of claim 40, further comprising determining in the cfDNA sample a methylation ratio for at least one marker locus selected from the group consisting of SEQ ID NO: 4, SEQ ID NO: 5 and SEQ ID NO: 6.

44. The method of any one of claims 40-43, further comprising determining in the cfDNA sample a methylation ratio for at least one tissue-specificity marker locus selected from the group consisting of:
(i) one or more tissue specificity marker locus selected from SEQ ID NO: 5 and SEQ ID NO: 8;
(ii) one or more tissue specificity marker locus selected from SEQ ID NO: 9 and SEQ ID NO: 10;
(iii) one or more tissue specificity marker locus selected from SEQ ID NO: 11 and SEQ ID NO: 12; and
(iv) one or more tissue specificity marker locus selected from SEQ ID NO: 13, SEQ ID NO: 14 and SEQ ID NO: 15,
thereby profiling methylation of the cfDNA sample.

45. A method for quantifying cancer-related methylation changes in a cell-free DNA sample of a human subject, the method comprising:
determining in the cell-free DNA sample a methylation value for at least one marker locus selected from the group consisting of SEQ ID NO: 1, SEQ ID NO: 2 and SEQ ID NO: 3, optionally further determining a methylation value for at least one marker locus selected from the group consisting of SEQ ID NO: 4, SEQ ID NO: and SEQ ID NO: 6,
thereby quantifying cancer-related methylation changes in the cell-free DNA sample, indicative of the presence of cancer in the subject.

46. The method of claim 45, further comprising quantifying cancer-related methylation changes in the cell-free DNA sample which are indicative of the tissue source of the cancer, said quantifying comprises at least one of:
(i) determining in the cfDNA sample a methylation value for at least one tissue specificity marker locus distinguishing between lung cancer and colorectal cancer selected from SEQ ID NO: 5 and SEQ ID NO: 8, and comparing to corresponding lung cancer and colorectal cancer reference values;
(ii) determining in the cfDNA sample a methylation value for at least one marker locus distinguishing between lung cancer and liver cancer selected from SEQ ID NO: 9 and SEQ ID NO: 10, and comparing to corresponding lung cancer and liver cancer reference values;
(iii) determining in the cfDNA sample a methylation value for at least one marker locus distinguishing between lung cancer and breast cancer selected from SEQ ID NO: 11 and SEQ ID NO: 12, and comparing to corresponding lung cancer and breast cancer reference values; and
(iv) determining in the cfDNA sample a methylation value for at least one marker locus distinguishing between lung cancer and hematological cancer selected from SEQ ID NO: 13, SEQ ID NO: 14 and SEQ ID NO: 15, and comparing to corresponding lung cancer and hematological cancer reference values, thereby quantifying cancer-related methylation changes in the cell-free DNA sample, indicative of the tissue source of the cancer.

47. A method for indicating a cancer tissue source in a human subject with a positive assessment of having cancer, the method comprising analyzing the methylation of at least one tissue-specificity marker locus selected from:
(i) one or more tissue specificity marker locus distinguishing between lung cancer and colorectal cancer selected from SEQ ID NO: 5 and SEQ ID NO: 8;
(ii) one or more tissue specificity marker locus distinguishing between lung cancer and liver cancer selected from SEQ ID NO: 9 and SEQ ID NO: 10;
(iii) one or more tissue specificity marker locus distinguishing between lung cancer and breast cancer selected from SEQ ID NO: 11 and SEQ ID NO: 12; and
(iv) one or more tissue specificity marker locus distinguishing between lung cancer and hematological cancer selected from SEQ ID NO: 13, SEQ ID NO: 14 and SEQ ID NO: 15.

48. A method for assessing the presence of cancer other than lung cancer in a human subject, the method comprising:
(i) determining in a cell-free DNA (cfDNA) sample of the subject a methylation value for at least one marker locus hypermethylated in cancer DNA compared to non-cancer DNA selected from the group consisting of SEQ ID NO: 1, SEQ ID NO: 2, SEQ ID NO: 3, SEQ ID NO: 4, SEQ ID NO: 5 and SEQ ID NO: 6;
(ii) comparing the methylation value of each of said at least one marker locus to at least one reference methylation value selected from a cancer methylation value and a non-cancer methylation value, to determine the likelihood that the subject has cancer other than lung cancer.

49. A method for assessing the presence of cancer in a human subject and providing an indication of the tissue source of the cancer, comprising:
(i) determining in a cell-free DNA (cfDNA) sample of the subject a methylation value for at least one marker locus hypermethylated in cancer DNA compared to non-cancer DNA selected from the group consisting of SEQ ID NO: 4, SEQ ID NO: 5 and SEQ ID NO: 6, and a methylation value for at least one tissue specificity marker locus selected from the group consisting of SEQ ID NOs: 5, 8-15;
(ii) assessing the presence of cancer in the subject based on the methylation value of the at least one marker locus hypermethylated in cancer DNA compared to non-cancer DNA, wherein the assessment comprises comparing the methylation value of each of said at least one marker locus to at least one reference methylation value selected from a cancer methylation value and a non-cancer methylation value; and
(iii) providing an indication of the tissue source of the cancer for a subject with a positive assessment of having cancer based on the methylation value of the at least one tissue specificity marker locus, wherein: SEQ ID NO: 5 and SEQ ID NO: 8 distinguish between lung cancer and colorectal cancer; SEQ ID NO: 9 and SEQ ID NO: 10 distinguish between lung cancer and liver cancer; SEQ ID NO: 11 and SEQ ID NO: 12 distinguish between lung cancer and breast cancer; and SEQ ID NO: 13, SEQ ID NO: 14 and SEQ ID NO: 15 distinguish between lung cancer and hematological cancer.

50. A method for treating lung, colon, breast, bladder, liver, or renal cell cancer in a patient, comprising:
(i) identifying the patient as being a lung, colon, breast, bladder, liver, or renal cell cancer patient by the method of any one of claims 1-49;
(ii) determining a mutational status of one or more genes selected from the group of KRAS, EGFR, ALK, ROS1, BRAF, RET, MET and NTRK in a tumor DNA sample from the patient; and
(iii) administering a therapy targeted to the mutational status of the patient's tumor DNA.

51. A method for treating occult or stage 0 lung cancer in a patient, comprising (i) identifying the patient as being a lung cancer patient by the method of one of claims 1-49; and
(ii) administering active lung cancer surveillance to said patient, wherein the active surveillance comprises one or more of a pulmonary imaging study, a sputum cytology study, a bronchoalveolar lavage cytology study, a bronchothoracic biopsy, and a transthoracic biopsy.

52. A method for performing computerized tomography (CT) on a patient, comprising:
(i) identifying the patient as being a lung cancer patient by the method of one of claims 1-49; and
(ii) performing a low-dose computed tomography (LDCT) scan on the chest of the patient.

53. A method for treating lung, breast cancer in a patient, comprising:
(i) identifying the patient as being a lung, breast, or colon cancer patient by the method of one of claims 1-49;
(ii) determining a PD-L1 status and optionally a mutational burden status in a tumor sample from the patient; and
(iii) assigning immunotherapy targeted to the PD-L1 status and optionally the mutational burden status of the tumor sample.

54. A method for treating a breast cancer patient, comprising (i) identifying the patient as being a breast cancer patient by the method of one of claims 1-49;
(ii) determining an ER, a HER2 and a BRCA1/2 status in a tissue sample from the patient; and
(iii) administering a therapy targeted to the ER, HER2 and BRCA1/2 status of the patient.

55. A method for treating a colon cancer patient, comprising:
(i) identifying the patient as being a colon cancer patient by the method of one of claims 1-49;
(ii) determining a mismatch repair (MMR) or microsatellite instability (MSI) status in a tumor sample from the patient; and
(iii) administering a therapy targeted to the MMR or MSI status of the patient.


SEQUENCE LISTING (195 sequences)

SEQ ID NO: 1 - DNA, 96 bp, Homo sapiens
ccgcggcgcccccgcgtgtccccgcagccactccgcggaagcagcggcgggagcgcaccaccttcacgcgttcacagctggacgtgctcgaggcgc

SEQ ID NO: 2 - DNA, 34 bp, Homo sapiens
gcgcaagcgcgccccaagaccattctcttcgcgc

SEQ ID NO: 3 - DNA, 59 bp, Homo sapiens
gcggcggccctaagacgacgcgggggtccttgtgcgcacttaaatggagtcggctgcgc

SEQ ID NO: 4 - DNA, 40 bp, Homo sapiens
ccgcagcgcccgccacacacccgcgccagaggtccagcgc

SEQ ID NO: 5 - DNA, 64 bp, Homo sapiens
gcgcggcgggcgacagccccccggataaccccgccgagggaggggcgcttgtaaaaccgagcgg

SEQ ID NO: 6 - DNA, 89 bp, Homo sapiens
ccgccgggtaaattagcggcgagcctcgccagacgctttcctccttgccttctttcgccgaaagggggcgcgctcctcccaggctgcgc

SEQ ID NO: 7 - DNA, 60 bp, Homo sapiens
agactaacttttctcttgtacagaatcatcaggctaaatttttggcattatttcagtcct

SEQ ID NO: 8 - DNA, 27 bp, Homo sapiens
gcgcccgcgcccccattggccgtgcgc

SEQ ID NO: 9 - DNA, 57 bp, Homo sapiens
gcggataagggtgtgcgcgagagccaatcaaaagcggatgtcgaaaagaggcgccgc

SEQ ID NO: 10 - DNA, 80 bp, Homo sapiens
gcgcggtgaacgtgggggttgaaacgctccacgcggaaggtagagggcaggggccaagggggcgatcctggtggctgcgc

SEQ ID NO: 11 - DNA, 47 bp, Homo sapiens
ccgcggagctgcaagggggggcggtttctcacccgccccgagagcgc

SEQ ID NO: 12 - DNA, 65 bp, Homo sapiens
gcgctttcttgggtcccccattcccccaggttagagcgcggctccaggaacctatgtccgcgcgg

SEQ ID NO: 13 - DNA, 68 bp, Homo sapiens
gcgcttgcgcccggttcaggaccgggtccaaatgaggttggcgtgcgccgtgcaggtgaggagtgcgg

SEQ ID NO: 14 - DNA, 58 bp, Homo sapiens
ccgcttccccggtctttgccaagtgggtttacgaggtctgaggcgtgttggggcgcgc

SEQ ID NO: 15 - DNA, 65 bp, Homo sapiens
gcgggcgggctgcgcacagcggggacaaggctgcccccttcctcctccgctgcctccgcggccgc

SEQ ID NO: 16 - DNA, 17 bp, Artificial Sequence
  Primer
gacccgcggcgcccccg

SEQ ID NO: 17 - DNA, 18 bp, Artificial Sequence
  Primer
atgacccgcggcgccccc

SEQ ID NO: 18 - DNA, 19 bp, Artificial Sequence
  Primer
caatgacccgcggcgcccc

SEQ ID NO: 19 - DNA, 19 bp, Artificial Sequence
  Primer
caccaatgacccgcggcgc

SEQ ID NO: 20 - DNA, 19 bp, Artificial Sequence
  Primer
gccaccaatgacccgcggc

SEQ ID NO: 21 - DNA, 18 bp, Artificial Sequence
  Primer
cgtggccaccaatgaccc

SEQ ID NO: 22 - DNA, 18 bp, Artificial Sequence
  Primer
gcgcaagcgcgccccaag

SEQ ID NO: 23 - DNA, 20 bp, Artificial Sequence
  Primer
cacctcggtcctcgggtaga

SEQ ID NO: 24 - DNA, 20 bp, Artificial Sequence
  Primer
ccccacctcggtcctcgggt

SEQ ID NO: 25 - DNA, 20 bp, Artificial Sequence
  Primer
aatccccacctcggtcctcg

SEQ ID NO: 26 - DNA, 20 bp, Artificial Sequence
  Primer
cgaatccccacctcggtcct

SEQ ID NO: 27 - DNA, 20 bp, Artificial Sequence
  Primer
tgtcgaatccccacctcggt

SEQ ID NO: 28 - DNA, 19 bp, Artificial Sequence
  Primer
gcggcggccctaagacgac

SEQ ID NO: 29 - DNA, 19 bp, Artificial Sequence
  Primer
agcggcggccctaagacga

SEQ ID NO: 30 - DNA, 20 bp, Artificial Sequence
  Primer
aaagcggcggccctaagacg

SEQ ID NO: 31 - DNA, 18 bp, Artificial Sequence
  Primer
taaagcggcggccctaag

SEQ ID NO: 32 - DNA, 18 bp, Artificial Sequence
  Primer
agtaaagcggcggcccta

SEQ ID NO: 33 - DNA, 21 bp, Artificial Sequence
  Primer
atagtaaagcggcggccctaa

SEQ ID NO: 34 - DNA, 17 bp, Artificial Sequence
  Primer
ccgcagcgcccgccaca

SEQ ID NO: 35 - DNA, 18 bp, Artificial Sequence
  Primer
tcccgcagcgcccgccac

SEQ ID NO: 36 - DNA, 19 bp, Artificial Sequence
  Primer
ggtcccgcagcgcccgcca

SEQ ID NO: 37 - DNA, 20 bp, Artificial Sequence
  Primer
cacggtcccgcagcgcccgc

SEQ ID NO: 38 - DNA, 19 bp, Artificial Sequence
  Primer
gctcacggtcccgcagcgc

SEQ ID NO: 39 - DNA, 19 bp, Artificial Sequence
  Primer
acagctcacggtcccgcag

SEQ ID NO: 40 - DNA, 15 bp, Artificial Sequence
  Primer
gatagcgcggcgggc

SEQ ID NO: 41 - DNA, 16 bp, Artificial Sequence
  Primer
tcggatagcgcggcgg

SEQ ID NO: 42 - DNA, 17 bp, Artificial Sequence
  Primer
gatcggatagcgcggcg

SEQ ID NO: 43 - DNA, 18 bp, Artificial Sequence
  Primer
agggatcggatagcgcgg

SEQ ID NO: 44 - DNA, 17 bp, Artificial Sequence
  Primer
gccagggatcggatagc

SEQ ID NO: 45 - DNA, 20 bp, Artificial Sequence
  Primer
agcagccagggatcggatag

SEQ ID NO: 46 - DNA, 19 bp, Artificial Sequence
  Primer
ccgccgggtaaattagcgg

SEQ ID NO: 47 - DNA, 19 bp, Artificial Sequence
  Primer
tccgccgggtaaattagcg

SEQ ID NO: 48 - DNA, 19 bp, Artificial Sequence
  Primer
ttccgccgggtaaattagc

SEQ ID NO: 49 - DNA, 19 bp, Artificial Sequence
  Primer
tttccgccgggtaaattag

SEQ ID NO: 50 - DNA, 19 bp, Artificial Sequence
  Primer
ctttccgccgggtaaatta

SEQ ID NO: 51 - DNA, 21 bp, Artificial Sequence
  Primer
gctttccgccgggtaaattag

SEQ ID NO: 52 - DNA, 23 bp, Artificial Sequence
  Primer
agcaaggtgaagactaacttttc

SEQ ID NO: 53 - DNA, 23 bp, Artificial Sequence
  Primer
cttgtacagaatcatcaggctaa

SEQ ID NO: 54 - DNA, 25 bp, Artificial Sequence
  Primer
gtccttggagacatctgagagattc

SEQ ID NO: 55 - DNA, 20 bp, Artificial Sequence
  Primer
gacatctgagagattccggg

SEQ ID NO: 56 - DNA, 22 bp, Artificial Sequence
  Primer
caaagaaagcaaggtgaagact

SEQ ID NO: 57 - DNA, 24 bp, Artificial Sequence
  Primer
cagtccttggagacatctgagaga

SEQ ID NO: 58 - DNA, 21 bp, Artificial Sequence
  Primer
cagtcacctaataagttgccg

SEQ ID NO: 59 - DNA, 21 bp, Artificial Sequence
  Primer
acagtcacctaataagttgcc

SEQ ID NO: 60 - DNA, 22 bp, Artificial Sequence
  Primer
tacagtcacctaataagttgcc

SEQ ID NO: 61 - DNA, 20 bp, Artificial Sequence
  Primer
gtacagtcacctaataagtt

SEQ ID NO: 62 - DNA, 21 bp, Artificial Sequence
  Primer
agtacagtcacctaataagtt

SEQ ID NO: 63 - DNA, 23 bp, Artificial Sequence
  Primer
aagtacagtcacctaataagttg

SEQ ID NO: 64 - DNA, 18 bp, Artificial Sequence
  Primer
ctggtcctgcggataagg

SEQ ID NO: 65 - DNA, 18 bp, Artificial Sequence
  Primer
actggtcctgcggataag

SEQ ID NO: 66 - DNA, 19 bp, Artificial Sequence
  Primer
gactggtcctgcggataag

SEQ ID NO: 67 - DNA, 18 bp, Artificial Sequence
  Primer
tgactggtcctgcggata

SEQ ID NO: 68 - DNA, 18 bp, Artificial Sequence
  Primer
atgactggtcctgcggat

SEQ ID NO: 69 - DNA, 21 bp, Artificial Sequence
  Primer
gatgactggtcctgcggataa

SEQ ID NO: 70 - DNA, 18 bp, Artificial Sequence
  Primer
tgcgtcttgtatcacctg

SEQ ID NO: 71 - DNA, 19 bp, Artificial Sequence
  Primer
ctgcgtcttgtatcacctg

SEQ ID NO: 72 - DNA, 20 bp, Artificial Sequence
  Primer
actgcgtcttgtatcacctg

SEQ ID NO: 73 - DNA, 20 bp, Artificial Sequence
  Primer
aactgcgtcttgtatcacct

SEQ ID NO: 74 - DNA, 20 bp, Artificial Sequence
  Primer
gaactgcgtcttgtatcacc

SEQ ID NO: 75 - DNA, 21 bp, Artificial Sequence
  Primer
agaactgcgtcttgtatcacc

SEQ ID NO: 76 - DNA, 17 bp, Artificial Sequence
  Primer
ctgggcgaaggcgttgg

SEQ ID NO: 77 - DNA, 18 bp, Artificial Sequence
  Primer
cctgggcgaaggcgttgg

SEQ ID NO: 78 - DNA, 19 bp, Artificial Sequence
  Primer
ccctgggcgaaggcgttgg

SEQ ID NO: 79 - DNA, 19 bp, Artificial Sequence
  Primer
cccctgggcgaaggcgttg

SEQ ID NO: 80 - DNA, 19 bp, Artificial Sequence
  Primer
acccctgggcgaaggcgtt

SEQ ID NO: 81 - DNA, 20 bp, Artificial Sequence
  Primer
tacccctgggcgaaggcgtt

SEQ ID NO: 82 - DNA, 18 bp, Artificial Sequence
  Primer
gcgctttcttgggtcccc

SEQ ID NO: 83 - DNA, 18 bp, Artificial Sequence
  Primer
ctgcgctttcttgggtcc

SEQ ID NO: 84 - DNA, 19 bp, Artificial Sequence
  Primer
ctctgcgctttcttgggtc

SEQ ID NO: 85 - DNA, 19 bp, Artificial Sequence
  Primer
tctgctctgcgctttcttg

SEQ ID NO: 86 - DNA, 20 bp, Artificial Sequence
  Primer
gctctgctctgcgctttctt

SEQ ID NO: 87 - DNA, 20 bp, Artificial Sequence
  Primer
ctagctctgctctgcgcttt

SEQ ID NO: 88 - DNA, 20 bp, Artificial Sequence
  Primer
gcgcttgcgcccggttcagg

SEQ ID NO: 89 - DNA, 20 bp, Artificial Sequence
  Primer
gagcgcttgcgcccggttca

SEQ ID NO: 90 - DNA, 19 bp, Artificial Sequence
  Primer
ttgagcgcttgcgcccggt

SEQ ID NO: 91 - DNA, 19 bp, Artificial Sequence
  Primer
tgttgagcgcttgcgcccg

SEQ ID NO: 92 - DNA, 20 bp, Artificial Sequence
  Primer
tcctggatgttgagcgcttg

SEQ ID NO: 93 - DNA, 21 bp, Artificial Sequence
  Primer
attcctggatgttgagcgctt

SEQ ID NO: 94 - DNA, 21 bp, Artificial Sequence
  Primer
ccgcttccccggtctttgcca

SEQ ID NO: 95 - DNA, 20 bp, Artificial Sequence
  Primer
ccgcttccccggtctttgcc

SEQ ID NO: 96 - DNA, 19 bp, Artificial Sequence
  Primer
ccgcttccccggtctttgc

SEQ ID NO: 97 - DNA, 17 bp, Artificial Sequence
  Primer
ccgcttccccggtcttt

SEQ ID NO: 98 - DNA, 19 bp, Artificial Sequence
  Primer
gccgcttccccggtctttg

SEQ ID NO: 99 - DNA, 19 bp, Artificial Sequence
  Primer
agccgcttccccggtcttt

SEQ ID NO: 100 - DNA, 21 bp, Artificial Sequence
  Primer
tcggcgacttccccgaagacc

SEQ ID NO: 101 - DNA, 21 bp, Artificial Sequence
  Primer
actcggcgacttccccgaaga

SEQ ID NO: 102 - DNA, 21 bp, Artificial Sequence
  Primer
ccaactcggcgacttccccga

SEQ ID NO: 103 - DNA, 20 bp, Artificial Sequence
  Primer
cgtttcctggaactccaact

SEQ ID NO: 104 - DNA, 20 bp, Artificial Sequence
  Primer
ttatcgtttcctggaactcc

SEQ ID NO: 105 - DNA, 22 bp, Artificial Sequence
  Primer
ttatcgtttcctggaactccaa

SEQ ID NO: 106 - DNA, 20 bp, Artificial Sequence
  Primer
atgtcagggtagcgagtctt

SEQ ID NO: 107 - DNA, 20 bp, Artificial Sequence
  Primer
tgtcagggtagcgagtcttg

SEQ ID NO: 108 - DNA, 20 bp, Artificial Sequence
  Primer
gtcagggtagcgagtcttgg

SEQ ID NO: 109 - DNA, 20 bp, Artificial Sequence
  Primer
tcagggtagcgagtcttggc

SEQ ID NO: 110 - DNA, 21 bp, Artificial Sequence
  Primer
tcagggtagcgagtcttggcg

SEQ ID NO: 111 - DNA, 20 bp, Artificial Sequence
  Primer
atgtcagggtagcgagtctt

SEQ ID NO: 112 - DNA, 20 bp, Artificial Sequence
  Primer
atgtcaagtgaaagtgaaga

SEQ ID NO: 113 - DNA, 21 bp, Artificial Sequence
  Primer
atgtcaagtgaaagtgaagaa

SEQ ID NO: 114 - DNA, 20 bp, Artificial Sequence
  Primer
gtcaagtgaaagtgaagaat

SEQ ID NO: 115 - DNA, 20 bp, Artificial Sequence
  Primer
tcaagtgaaagtgaagaata

SEQ ID NO: 116 - DNA, 21 bp, Artificial Sequence
  Primer
tcaagtgaaagtgaagaataa

SEQ ID NO: 117 - DNA, 24 bp, Artificial Sequence
  Primer
atgtcaagtgaaagtgaagaataa

SEQ ID NO: 118 - DNA, 20 bp, Artificial Sequence
  Primer
atctaccgggtgtgggaact

SEQ ID NO: 119 - DNA, 21 bp, Artificial Sequence
  Primer
atctaccgggtgtgggaactc

SEQ ID NO: 120 - DNA, 21 bp, Artificial Sequence
  Primer
tctaccgggtgtgggaactca

SEQ ID NO: 121 - DNA, 20 bp, Artificial Sequence
  Primer
taccgggtgtgggaactcac

SEQ ID NO: 122 - DNA, 21 bp, Artificial Sequence
  Primer
taccgggtgtgggaactcacc

SEQ ID NO: 123 - DNA, 21 bp, Artificial Sequence
  Primer
tgccttctgagtaaggcagtt

SEQ ID NO: 124 - DNA, 24 bp, Artificial Sequence
  Primer
ccgatcgaattcccgggctaggcc

SEQ ID NO: 125 - DNA, 24 bp, Artificial Sequence
  Primer
gatcgaattcccgggctaggccat

SEQ ID NO: 126 - DNA, 24 bp, Artificial Sequence
  Primer
tcgaattcccgggctaggccattt

SEQ ID NO: 127 - DNA, 24 bp, Artificial Sequence
  Primer
aattcccgggctaggccatttcac

SEQ ID NO: 128 - DNA, 24 bp, Artificial Sequence
  Primer
tcccgggctaggccatttcactgc

SEQ ID NO: 129 - DNA, 21 bp, Artificial Sequence
  Primer
cgtgctttgccgatcgaattc

SEQ ID NO: 130 - DNA, 23 bp, Artificial Sequence
  Primer
atgttcgagtcggtcgtaacgag

SEQ ID NO: 131 - DNA, 23 bp, Artificial Sequence
  Primer
tgttcgagtcggtcgtaacgagg

SEQ ID NO: 132 - DNA, 23 bp, Artificial Sequence
  Primer
gttcgagtcggtcgtaacgaggc

SEQ ID NO: 133 - DNA, 23 bp, Artificial Sequence
  Primer
ttcgagtcggtcgtaacgaggcc

SEQ ID NO: 134 - DNA, 24 bp, Artificial Sequence
  Primer
ttcgagtcggtcgtaacgaggccg

SEQ ID NO: 135 - DNA, 21 bp, Artificial Sequence
  Primer
atgttcgagtcggtcgtaacg

SEQ ID NO: 136 - DNA, 19 bp, Artificial Sequence
  Primer
gcaggataggtaccagcgc

SEQ ID NO: 137 - DNA, 19 bp, Artificial Sequence
  Primer
ggcaggataggtaccagcg

SEQ ID NO: 138 - DNA, 19 bp, Artificial Sequence
  Primer
aggcaggataggtaccagc

SEQ ID NO: 139 - DNA, 19 bp, Artificial Sequence
  Primer
aaggcaggataggtaccag

SEQ ID NO: 140 - DNA, 19 bp, Artificial Sequence
  Primer
gaaggcaggataggtacca

SEQ ID NO: 141 - DNA, 21 bp, Artificial Sequence
  Primer
tgaaggcaggataggtaccag

SEQ ID NO: 142 - DNA, 23 bp, Artificial Sequence
  Primer
tgaagtgaactattccttaggtg

SEQ ID NO: 143 - DNA, 23 bp, Artificial Sequence
  Primer
gtctccaaggactgaaataatgc

SEQ ID NO: 144 - DNA, 28 bp, Artificial Sequence
  Primer
gagtagcaaaaaatagctgaagtgaact

SEQ ID NO: 145 - DNA, 23 bp, Artificial Sequence
  Primer
tgacaaccaatgagtagcaaaaa

SEQ ID NO: 146 - DNA, 20 bp, Artificial Sequence
  Primer
gttgtcagtgtggccagaga

SEQ ID NO: 147 - DNA, 21 bp, Artificial Sequence
  Primer
tgttgtcagtgtggccagaga

SEQ ID NO: 148 - DNA, 19 bp, Artificial Sequence
  Primer
ggacgggcacgtgacgcgc

SEQ ID NO: 149 - DNA, 19 bp, Artificial Sequence
  Primer
tggacgggcacgtgacgcg

SEQ ID NO: 150 - DNA, 19 bp, Artificial Sequence
  Primer
ctggacgggcacgtgacgc

SEQ ID NO: 151 - DNA, 18 bp, Artificial Sequence
  Primer
ctggacgggcacgtgacg

SEQ ID NO: 152 - DNA, 18 bp, Artificial Sequence
  Primer
tctggacgggcacgtgac

SEQ ID NO: 153 - DNA, 22 bp, Artificial Sequence
  Primer
ttaatagcgtgcggagtgattt

SEQ ID NO: 154 - DNA, 20 bp, Artificial Sequence
  Primer
gacgtgactggctggagcgg

SEQ ID NO: 155 - DNA, 20 bp, Artificial Sequence
  Primer
cgacgtgactggctggagcg

SEQ ID NO: 156 - DNA, 20 bp, Artificial Sequence
  Primer
gcgacgtgactggctggagc

SEQ ID NO: 157 - DNA, 20 bp, Artificial Sequence
  Primer
cgcgacgtgactggctggag

SEQ ID NO: 158 - DNA, 20 bp, Artificial Sequence
  Primer
tcgcgacgtgactggctgga

SEQ ID NO: 159 - DNA, 20 bp, Artificial Sequence
  Primer
acctcgcgacgtgactggct

SEQ ID NO: 160 - DNA, 20 bp, Artificial Sequence
  Primer
cagcaaatagcaaaaagcgc

SEQ ID NO: 161 - DNA, 20 bp, Artificial Sequence
  Primer
gcagcaaatagcaaaaagcg

SEQ ID NO: 162 - DNA, 20 bp, Artificial Sequence
  Primer
ggcagcaaatagcaaaaagc

SEQ ID NO: 163 - DNA, 20 bp, Artificial Sequence
  Primer
cggcagcaaatagcaaaaag

SEQ ID NO: 164 - DNA, 20 bp, Artificial Sequence
  Primer
tcggcagcaaatagcaaaaa

SEQ ID NO: 165 - DNA, 21 bp, Artificial Sequence
  Primer
cgtcggcagcaaatagcaaaa

SEQ ID NO: 166 - DNA, 20 bp, Artificial Sequence
  Primer
gaagaagggaggcctggcgc

SEQ ID NO: 167 - DNA, 20 bp, Artificial Sequence
  Primer
agaagaagggaggcctggcg

SEQ ID NO: 168 - DNA, 20 bp, Artificial Sequence
  Primer
cagaagaagggaggcctggc

SEQ ID NO: 169 - DNA, 20 bp, Artificial Sequence
  Primer
tcagaagaagggaggcctgg

SEQ ID NO: 170 - DNA, 19 bp, Artificial Sequence
  Primer
tcagaagaagggaggcctg

SEQ ID NO: 171 - DNA, 21 bp, Artificial Sequence
  Primer
aatcatggctcgtgacgactc

SEQ ID NO: 172 - DNA, 20 bp, Artificial Sequence
  Primer
agccgtccctactacaccgc

SEQ ID NO: 173 - DNA, 20 bp, Artificial Sequence
  Primer
tagccgtccctactacaccg

SEQ ID NO: 174 - DNA, 20 bp, Artificial Sequence
  Primer
ttagccgtccctactacacc

SEQ ID NO: 175 - DNA, 20 bp, Artificial Sequence
  Primer
tttagccgtccctactacac

SEQ ID NO: 176 - DNA, 20 bp, Artificial Sequence
  Primer
atttagccgtccctactaca

SEQ ID NO: 177 - DNA, 21 bp, Artificial Sequence
  Primer
ccatttagccgtccctactac

SEQ ID NO: 178 - DNA, 20 bp, Artificial Sequence
  Primer
tttggggggaggtgacccgc

SEQ ID NO: 179 - DNA, 20 bp, Artificial Sequence
  Primer
ttttggggggaggtgacccg

SEQ ID NO: 180 - DNA, 20 bp, Artificial Sequence
  Primer
cttttggggggaggtgaccc

SEQ ID NO: 181 - DNA, 20 bp, Artificial Sequence
  Primer
tcttttggggggaggtgacc

SEQ ID NO: 182 - DNA, 20 bp, Artificial Sequence
  Primer
gtcttttggggggaggtgac

SEQ ID NO: 183 - DNA, 21 bp, Artificial Sequence
  Primer
aggttggcacagggtcttttg

SEQ ID NO: 184 - DNA, 20 bp, Artificial Sequence
  Primer
ttttgcgagaacgtgagcgc

SEQ ID NO: 185 - DNA, 20 bp, Artificial Sequence
  Primer
cttttgcgagaacgtgagcg

SEQ ID NO: 186 - DNA, 20 bp, Artificial Sequence
  Primer
ccttttgcgagaacgtgagc

SEQ ID NO: 187 - DNA, 20 bp, Artificial Sequence
  Primer
gccttttgcgagaacgtgag

SEQ ID NO: 188 - DNA, 19 bp, Artificial Sequence
  Primer
gccttttgcgagaacgtga

SEQ ID NO: 189 - DNA, 21 bp, Artificial Sequence
  Primer
aggagccttttgcgagaacgt

SEQ ID NO: 190 - DNA, 19 bp, Artificial Sequence
  Primer
cagactgagatagacgcgg

SEQ ID NO: 191 - DNA, 19 bp, Artificial Sequence
  Primer
tcagactgagatagacgcg

SEQ ID NO: 192 - DNA, 19 bp, Artificial Sequence
  Primer
gtcagactgagatagacgc

SEQ ID NO: 193 - DNA, 19 bp, Artificial Sequence
  Primer
agtcagactgagatagacg

SEQ ID NO: 194 - DNA, 19 bp, Artificial Sequence
  Primer
tagtcagactgagatagac

SEQ ID NO: 195 - DNA, 21 bp, Artificial Sequence
  Primer
gtagtcagactgagatagacg